(19) United States
(12) Patent Application Publication
(10) Pub. No.: US 2005/0054103 A1
(43) Pub. Date: Mar. 10, 2005

(54) Expansion of renewable stem cell populations using modulators of PI 3-kinase

(76) Inventors: Tony Peled, Mevaseret Zion (IL); Frida Grynspan, Mevasseret Zion (IL)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/795,215

(22) Filed: Mar. 4, 2004

Related U.S. Application Data
(63) Continuation-in-part of application No. PCT/IL03/00235, filed on Mar. 18, 2003.
(63) Continuation-in-part of application No. PCT/IL03/00681, filed on Aug. 17, 2003.
(60) Provisional application No. 60/452,545, filed on Mar. 7, 2003.

(51) Int. Cl. C12N 5/08; C12N 15/85

(52) U.S. Cl. 435/455; 435/372

(57) ABSTRACT

Provided are ex vivo and in vivo methods of expanding renewable stem cells using modulators of PI 3-kinase activity, expanded populations of renewable stem cells, and uses thereof.

### RELATED APPLICTIONS

This application claims priority to U.S. Provisional Patent Application No. 60/452,545, filed Mar. 7, 2003. This application also claims priority to, and is a continuation in part of, PCT/IL03/00235, filed Mar. 18, 2003, and PCT/IL03/00681, filed Aug. 17, 2003.

The contents of all of these applications are hereby incorporated by reference in their entireties.

### FIELD AND BACKGROUND OF THE INVENTION

The present invention relates to methods of expansion of renewable stem cells, to expanded populations of renewable stem cells and to their uses. In particular, ex-vivo and/or in-vivo stem cell expansion is achieved according to the present invention by downregulation of a Phosphatidylinositol 3-kinase (PI 3-kinase) signaling pathway, either at the protein level via PI 3-kinase inhibitors, such as, for example, wortmannin and LY294002, or at the expression level via genetic engineering techniques, such as small interfering RNA (siRNA), ribozyme, and antisense techniques.

The present invention further relates to therapeutic applications in which these methods and/or the expanded stem cells populations obtained thereby are utilized.

An increasing need for ex-vivo cultures of hematopoietic and non-hematopoietic stem cells has arisen, in particular for purposes such as stem cell expansion and retroviral-mediated gene transduction. Methods for generating ex-vivo cultures of stem cells to date, however, result in a rapid decline in stem cell population activity, further resulting in a markedly impaired self renewal potential and diminished transplantability of the cultured cell populations. The need to improve such methods is obvious. Additionally, applications in gene therapy using retroviral vectors necessitate the use of proliferating hematopoietic stem cells, yet require that these cells remain undifferentiated while in culture, in order to maintain long-term expression of the transduced gene. Thus, the ability to maintain ex-vivo cultures of hematopoietic and non-hematopoietic stem cell populations with long-term, self-renewal capacity is of critical importance for a wide array of medical therapeutic applications.

Presently, expansion of renewable stem cells have been achieved either by growing the stem cells over a feeder layer of fibroblast cells, or by growing the cells in the presence of the early acting cytokines thrombopoietin (TPO), interleukin-6 (IL-6), an FLT-3 ligand and stem cell factor (SCF) (Madlambayan G J et al. (2001) J Hematother Stem Cell Res 10: 481, Punzel M et al. (1999) Leukemia 13: 92, and Lange W et al. (1996) Leukemia 10: 943). While expanding stem cells over a feeder layer results in vast, substantially endless cell expansion, expanding stem cells without a feeder layer, in the presence of the early acting cytokines, results in an elevated degree of differentiation (see controls described in the Examples section and Leslie N R et al. (Blood (1998) 92: 4798), Petzer A L et al. (1996) J Exp Med June 183: 2551, Kawa Y et al. (2000) Pigment Cell Res 8: 73).

In any case, using present day technology, stem cells cannot be expanded unless first substantially enriched or isolated to homogeneity.

The art presently fails to teach an efficient method for expansion of renewable stem cells without a feeder layer.

CD38

CD38 is a member of an emerging family of cytosolic and membrane-bound enzymes whose substrate is nicotinamide adenine dinucleotide (NAD), a coenzyme ubiquitously distributed in nature. In human, CD38 is a 45 kDa type II trans-membrane glycoprotein. Recently, it has been demonstrated that CD38 is a multifunctional enzyme that exerts both NAD⁺ glycohydrolase activity and ADP-ribosyl cyclase activity and is thus able to produce nicotinamide, ADP-ribose (ADPR), cyclic-ADPR (cADPR) and nicotinic acid adenine dinucleotide phosphate (NAADP) from its substrates (Howard et al., 1993 Science 252:1056-1059; Lee et al., 1999 Biol. Chem. 380;785-793). The soluble domain of human CD38 catalyzes the conversion of NAD⁺ to cyclic ADP-ribose and to ADP-ribose via a common covalent intermediate (Sauve, A. A., Deng, H. T., Angelletti, R. H., and Schramm, V. L. (2000) J. Am. Chem. Soc. 122, 7855-7859).

However, it was further found that CD38 is not characterized only by multi enzymatic activity but is further able to mobilize calcium, to transduce signals and to adhere to hyaluronan and to other ligands. Interaction with CD38 on various leukocyte subpopulation has profound though diverse effects on their life-span (Funaro A, Malavasi F J Biol Regul Homeost Agents 1999 January-March;13(1):54-61 Human CD38, a surface receptor, an enzyme, an adhesion molecule and not a simple marker).

CD38 is widely expressed in both hematopoietic and non hematopoietically-derived cells. Homologues of CD38 have also been found to be expressed in mammalian stromal cells (Bst-1) and in cells isolated from the invertebrate Aplysia californica (Prasad G S, 1996, nature Structural Biol 3:957-964).

Two of the metabolites produced by CD38, cADPR and NAADP, have been shown to induce the release of intracellular calcium in cells isolated from tissues of plants, invertebrates and mammals, suggesting that these metabolites may be global regulators of calcium responses (Lee et al., 1999 Biol. Chem. 380;785-793). Both cADPR and NAADP are known to induce calcium release from calcium stores that are distinct from those controlled by Ip³ receptors (Clapper, D L et al., 1987, J. Biological Chem. 262:9561-9568).

Hence, CD38, being the best-characterized mammalian ADP-ribosyl cyclase, is postulated to be an important source of cyclic ADP-ribose in vivo.

Nucleoplasmic calcium ions (Ca⁺²) influence highly important nuclear functions such as gene transcription, apoptosis, DNA repair, topoisomerase activation and polymerase unfolding. Although both inositol trisphosphate receptors and ryanodine receptors, which are types of Ca⁺² channel, are present in the nuclear membrane, their role in the homeostasis of nuclear Ca⁺² is still unclear.

It was found that CD38/ADP-ribosyl cyclase has its catalytic site within the nucleoplasm and hence it catalyses the intranuclear cyclization of NAD⁺, to produce nucleoplasmic cADPR. The latter activates ryanodine receptors of the inner nuclear membrane to trigger nucleoplasmic Ca⁺² release (Adebanjo O A et al. Nat Cell Biol 1999 November;1(7):409-14 A new function for CD38/ADP-ribosyl cyclase in nuclear Ca2+ homeostasis).

It was further found that agonists of ryanodine receptors sensitize cADPR-mediated calcium release and antagonists of ryanodine receptors block cADPR-dependent calcium release (Galione A et al., 1991, Science 253:143-146). Thus, it has been proposed that cADPR is likely to regulate calcium responses in tissues such as muscle and pancreas, where ryanodine receptors are expressed (Day et al., 2000 Parasitol 120:417-422; Silva et al., 1998, Biochem. Pharmacol 56:997-1003). It has been also shown that in mammalian smooth muscle cells, the calcium release in response to acetylcholine can be blocked not only with ryanodine receptor antagonists, but also with specific antagonists of cADPR such as 8-NH₂-cADPR or 8-Br-cADPR (Guse, A H, 1999, Cell. Signal. 11:309-316). These findings, as well as others, indicate that ryanodine receptor agonists/antagonists such as cADPR can regulate calcium responses in cells isolated from diverse species.

As is discussed hereinabove, self-renewal of hemopoietic stem and progenitor cells (HPC), both in vivo and in vitro, is limited by cell differentiation. Differentiation in the hematopoietic system involves, among other changes, altered expression of surface antigens (Sieff C, Bicknell D, Caine G, Robinson J, Lam G, Greaves M F (1982) Changes in cell surface antigen expression during hematopoietic differentiation. Blood 60:703). In normal human, most of the hematopoietic pluripotent stem cells and the lineage committed progenitor cells are CD34+. The majority of cells are CD34+CD38+, with a minority of cells (<10%) being CD34+CD38−. The CD34+CD38− phenotype appears to identify the most immature hematopoietic cells, which are capable of self-renewal and multilineage differentiation. The CD34+CD38− cell fraction contains more long-term culture initiating cells (LTC-IC) pre-CFU and exhibits longer maintenance of their phenotype and delayed proliferative response to cytokines as compared with CD34+CD38+ cells. CD34+CD38− can give rise to lymphoid and myeloid cells in vitro and have an enhanced capacity to repopulate SCID mice (Bhatia M, Wang J C Y, Kapp U, Bonnet D, Dick J E (1997) Purification of primitive human hematopoietic cells capable of repopulating immune-deficient mice. Proc Natl Acad Sci USA 94:5320). Moreover, in patients who received autologous blood cell transplantation, the number of CD34+CD38− cells infused correlated positively with the speed of hematopoietic recovery. In line with these functional features, CD34+CD38− cells have been shown to have detectable levels of telomerase.

Recently, it has been reported that granulocytic differentiation of human HL-60 cells (a committed cell line) can be induced by retinoic acid and is accompanied by a massive expression of CD38. Concomitant with CD38 expression was the accumulation of cADPR, and both time courses preceded the onset of differentiation, suggesting a causal role for CD38. Consistently, treatment of HL-60 cells with a permeant inhibitor of CD38, nicotinamide, inhibited both the CD38 activity and differentiation. More specific blockage of CD38 expression was achieved by using morpholino antisense oligonucleotides targeting its mRNA, which produced a corresponding inhibition of differentiation as well (Munshi C B, Graeff R, Lee H C, J Biol Chem 2002 Dec. 20;277(51):49453-8).

In view of the findings described above with respect to the effect of CD38 on cADPR and ryanodine signal transduction pathways and hence on cell expansion and differentiation, the present inventors have envisioned that by modulating the expression and/or the activity of CD38, the expansion and differentiation of stem cells could be controlled. In particular, it was hypothesized that by reducing the expression and/or the activity of CD38, using agents that downregulate the expression of CD38 or inhibit the activity thereof, expansion of renewable stem cells, devoid of differentiation, would be achievable.

Nicotinamide (NA) is a water-soluble derivative of vitamin B, whose physiological active forms are nicotinamide adenine dinucleotide (NAD+/NADH) and nicotinamide adenine dinucleotide phosphate (NADP+/NADPH). The physiological active forms of NA serve as coenzyme in a variety of important metabolic reactions. Nicotinamide is further known to inhibit the enzymatic activity of CD38, to thereby affect the cADPR signal transduction pathway, a feature which is demonstrated, for example, in the studies described hereinabove (see, for example, Munshi C B, Graeff R, Lee H C, J Biol Chem 2002 Dec. 20;277(51):49453-8).

Hence, while conceiving the present invention, it was hypothesized that nicotinamide, as well as other agents known to inhibit the enzymatic activity of CD38, can be utilized for expanding stem cell populations while inhibiting the differentiation of the stem cells. It was further hypothesized that other small molecules, which are capable of interfering, directly or indirectly, with the expression of CD38 can be similarly used.

Retinoic acid (RA), the natural acidic derivative of Vitamin A (retinol) is an important regulator of embryonic development and it also influences the growth and differentiation of a wide variety of adult cell types. The biological effects of RA are generally mediated through their interaction with specific ligand-activated nuclear transcription factors, their cognate RA receptors (RARs). Receptors of the retinoic acid family comprise RARs, RXRs, Vitamin D receptors (VDRs), thyroid hormone receptors (THRs) and others. When activated by specific ligands these receptors behave as transcription factors, controlling gene expression during embryonic and adult development. The RAR and RXR families of receptors uniquely exhibit modular structures harboring distinct DNA-binding and ligand-binding domains. These receptors probably mediate their biological effects by binding to regulatory elements (e.g., retinoic acid response elements, or RAREs) as RAR-RXR heterodimers that are present in the promoters of their specific target genes (1, 2, 3).

Retinoid receptors thus behave as ligand-dependent transcriptional regulators, repressing transcription in the absence of ligand and activating transcription in its presence. These divergent effects on transcription are mediated through the recruitment of co-regulators: un-liganded receptors bind corepressors (NCoR and SMRT) that are found within a complex exhibiting histone deacetylase (HDAC) activity, whereas liganded receptors recruit co-activators with histone acetylase activity (HATs). Chromatin remodeling may also be required, suggesting a hierarchy of promoter structure modifications in RA target genes carried out by multiple co-regulatory complexes.

The first retinoic acid receptor identified, designated RAR-alpha, modulates transcription of specific target genes in a manner which is ligand-dependent, as subsequently shown for many of the members of the steroid/thyroid hormone intracellular receptor superfamily. The endogenous low-molecular-weight ligand, upon which the transcription-modulating activity of RAR-alpha depends, is all-trans-retinoic acid. Retinoic acid receptor-mediated changes in gene expression result in characteristic alterations in cellular phenotype, affecting multiple tissues. Additional RAR-alpha related genes have been identified, designated RAR-beta and RAR-gamma, and exhibit a high level of homology to RAR-alpha and each other (4, 5). The ligand-binding region of the three RAR subtype receptors has a primary amino acid sequence divergence of less than 15%.

Similarly, additional members of the steroid/thyroid receptor superfamily responsive to retinoic acid have been identified (6), and have been designated as the retinoid X receptor (RXR) family. Like the RARs, the RXRs are also known to comprise at least three subtypes or isoforms, namely RXR-alpha, RXR-beta, and RXR-gamma, with corresponding unique patterns of expression (7).

Although both the RARs and RXRs bind the ligand all-trans-retinoic acid in vivo, the receptors differ in several important aspects. First, the RARs and RXRs significantly differ in their primary structure, especially regarding their ligand binding domains (e.g., alpha domains exhibit a mere 27% shared amino acid identity). These structural differences manifest in their differing relative degrees of responsiveness to various Vitamin A metabolites and synthetic retinoids. Additionally, tissue distribution patterns are distinctly different for RARs and RXRs. RARs and RXRs exhibit different target gene specificity. One example is regarding the cellular retinal binding protein type II (CRBPII) and apolipoprotein AI proteins that confer responsiveness to RXR, but not RAR. Furthermore, RAR has also been shown to repress RXR-mediated activation through the CRBPII RXR response element (8). These data indicate that the two separate retinoic acid responsive pathways are not simply redundant, but instead manifest a complex interplay.

Vitamin D (VitD) is an additional potent activator of one of the receptors belonging to the retinoid receptor superfamily. The nuclear hormone 1 alpha, 25-dihydroxyvitamin D (3) (1 alpha, 25 (OH) (2) D (3)) binds its cognate receptor (VDR) and acts as a transcription factor when in combined contact with the retinoid X receptor (RXR), coactivator proteins, and specific DNA binding sites (VDREs). Ligand-mediated conformational changes of the VDR comprise the molecular switch controlling nuclear 1 alpha, 25 (OH) (2) D (3), signaling events.

Cell-specific VDR antagonists reveal the exquisite control and regulation of the pleiotropic 1 alpha, 25 (OH) (2) D (3) endocrine system, with consequences in maintenance of calcium homeostasis, bone mineralization and other cellular functions. Antagonists to VitD were shown to act via the same mechanism: they selectively stabilize an antagonistic conformation of the ligand-binding domain of the VDR within VDR-RXR-VDRE complexes, inhibiting the interaction of the VDR with coactivator proteins and induction of transactivation. Interestingly, cells treated with VitD antagonists contain VDR-RXR heterodimers in different conformations as compared to cells stimulated with VitD agonists (16).

Retinoic acid and VitD can cooperatively stimulate transcriptional events involving a common DNA binding site or hormone response element (BRE). Conversely, VDR/RXR heterodimers have been found to bind without defmed polarity and in a transcriptionally unproductive manner to certain RA response elements, and under these circumstances Vitamin D inhibits the response to RA. Although competition for binding to DNA may contribute to this inhibitory response, titration of common coactivators by VDR also appears to be involved in this trans-repression. Therefore, the regulation of the transcriptional response to RA and VitD is dependent upon a complex combinatory pattern of interaction among the different receptors, co-activators (17) and their binding to the appropriate DNA binding sites.

In parallel to their function as transcriptional regulators, retinoid receptors such as RAR and RXR play important roles in regulating the growth and differentiation of a variety of cell-types, as well (18). RAR agonists such as all-trans-retinoic acid (ATRA) are predominantly known for their effects in inducing cell-differentiation, as seen in experiments utilizing malignant cancer cells and embryonic stem cells (19), where potent induction of terminal differentiation was evident. Cell differentiation is not an exclusive result, however, as RA has been shown to exhibit different effects on cultured hematopoietic cells, depending on their maturational state (20). While retinoids accelerated the growth and differentiation of granulocyte progenitors in cytokine-stimulated cultures of purified CD34⁺ cells, use of stem cells produced an opposite effect (42). Retinoid treatment has also been shown to inhibit differentiation of pre-adipose cells (43).

Whereas the RAR antagonist AGN 193109 exerted a positive effect on the differentiation of hematopoietic stem cells (41) the RAR agonist 4-[4-(4-ethylphenyl)dimethyl-chromen-yl]ethynyl}-benzoic acid] functions in an opposing manner. Conversely, RAR antagonists have been shown to prevent granulocytic differentiation in experiments utilizing the promyelocytic cell line, HL-60 (41). Similarly, creation of myeloid cell lines defective in signaling through their retinoid receptors do not undergo granulocytic differentiation in the presence of G-CSF (22), and retinoid-deficient tissues acquire a pre-malignant phenotype, and a concomitant loss of differentiation (29, 30). Malignant cell lines derived from various carcinomas exhibit diminished expression of retinoic acid receptor mRNA, implying that the loss of expression may be an important event in tumorogenesis (33, 34, 35, 36, 37). Furthermore, disruption of retinoic acid receptor activity, as evidenced in knock-out mouse models disrupted for the RAR gene, display an in vitro block to granulocytic differentiation (38, 39).

However, other studies using a similar approach have resulted in the development of hematopoietic cell lines (23). The hematopoietic stem and early progenitor cells are characterized by their surface expression of the surface antigen marker known as CD34⁺, and exclusion of expression of the surface lineage antigen markers, Lin⁻. Experiments utilizing several leukemia cell lines revealed that retinoic acid receptor mediated signaling results in the induction of expression of the differentiation marker CD38 cell surface antigen whereas antagonists to RAR abolished CD38 antigen up-regulation (24, 25).

Therefore, to date, the data are conflicting as to definitive roles for VitD and RA in induction of myelomonocytic and promyelocytic cell differentiation, or prevention of these processes. Although some previous studies with inactivation of RAR, RXR and VDR using antagonists, antisense technology or transduction methods with truncated receptors, yielded inhibited granulocytic and monocytic differentiation, these studies were conducted using leukemia cell lines that are blocked at the myeloblast or promyelocytic stage of differentiation (19, 22, 64).

PI 3-Kinase and CD38

PI 3-kinase is a lipid kinase composed of a Src homology 2 domain-containing regulatory subunit (p85) and a 110-kD catalytic subunit (p110). PI 3-kinase catalyzes the formation of inositol phospholipids phosphorylated at the D3 position of PIPI 3-kinase. PI 3-kinase activity has been linked to many aspects of cell transformation processes, including increased cell growth, proliferation, adhesion, metastasis and angiogenesis, and has been implicated in the pathogenesis of colorectal cancer, breast cancer, ovarian and cervical tumors, and proliferative and anti-apoptotic effects of estrogen in breast and other tissues (Fry, Breast Can Res 2001, 3:304-12, Bhat-Nakshatri et al, Br J Cancer 2004;90:853-9). It was shown that the PI 3-kinase inhibitors wortmannin and LY294002 prevented increase in CD38 mRNA expression and the overexpression of membrane CD38 antigen as well as that of, CD157, a CD38-related antigen on HL-60 and normal marrow CD34+ cells exposed to retinoic acid [Phosphatidylinositol 3-kinases are involved in the all-trans retinoic acid-induced upregulation of CD38 antigen on human haematopoietic cells. Lewandowski D, Linassier C, Iochmann S, Degenne M, Domenech J, Colombat P, Binet C, Herault O. Br J Haematol 2002 August: 118(2): 535-44)].

Downstream signal transduction imposed by nuclear receptors such as the RARs, RXRs and VDRs may also be inhibited by inhibition of PI 3-kinase, which is an obligatory factor for proper receptor signaling. The critical function of PI 3-kinase in the activation of nuclear receptors such as VDR was demonstrated in THP-1 cells. Treatment of THP-1 cells with 1α,25-dihydroxyvitamin D₃ (D₃) was associated with rapid and transient increases in PI 3-kinase activity, as well as, with maturation of myeloid cells and surface expressions of CD14 and CD11b, markers of cell differentiation. Induction of CD14 and CD11b expression in response to D₃ was abrogated by (a) the PI 3-kinase inhibitors LY294002 and wortmannin; (b) antisense oligonucleotides to mRNA for the p110 catalytic subunit of PI 3-kinase; and (c) a dominant negative mutant of PI 3-kinase. Similarly, LY294002 and wortmannin inhibited D₃-induced expression of both CD14 and CD11b in peripheral blood monocytes. Western blots and in vitro kinase assays carried out on immunoprecipitates of the VDR showed that D₃ treatment brought about formation of a complex containing both PI 3-kinase and the VDR. These findings reveal a novel, nongenomic mechanism of hormone action regulating monocyte differentiation, in which vitamin D₃ activates a VDR and PI 3-kinase-dependent signaling pathway [The Journal of Experimental Medicine, Volume 190, Number 11, Dec. 6, 1999 1583-1594; 5-Dihydroxyvitamin D₃-induced Myeloid Cell Differentiation Is Regulated by a Vitamin D Receptor-Phosphatidylinositol 3-Kinase Signaling Complex Zakaria Hmama, Devki Nandan, Laura Sly, Keith L. Knutson, Patricia Herrera-Velit, and Neil E. Reiner].

The functionality of PI 3-kinase as an obligatory downstream factor in the cellular pathways involved in induction of leukaemic cell differentiation was also demonstrated in HL-60 cells that were induced to granulocytic differentiation by all-trans-retinoic acid. Immunochemical and immunocytochemical analyses by confocal microscopy also reveal an increase in the amount of PI 3-kinase, which is particularly evident at the nuclear level. Inhibition of PI 3-kinase activity by nanomolar concentrations of wortmannin and of its expression by transfection with an antisense fragment of p85α prevented the differentiative process. Further, it was observed that inhibition of the PI 3-kinase signaling pathway by wortmannin treatment of HL-60 cells prior to differentiation with all-trans-retinoic acid is lethal, leading to apoptosis following differentation. These data indicate that PI 3-kinase activity plays an essential role in promoting granulocytic differentiation (Bertagnolo, et al., Cancer Research, 1999;59:542-546; and Ma et al, Cell Cycle 2004;3:67-70).

The involvement of PI 3-kinase in cell differentiation regulatory pathways was demonstrated also in non hematopoietic cells. Smooth Muscle Cells (SMC) de-differentiation is induced by PDGF-BB, bFGF and EGF, whereas IGF-I-triggered signaling pathway in maintaining a differentiated phenotype of gizzard SMC in culture. It was demonstrated that distinctly different signaling pathways regulate the SMC phenotype. Both the ERK and p38MAPK pathways triggered by PDGF-BB, bFGF, and EGF were found to play an essential role in inducing SMC de-differentiation, whereas the PI 3-kinase/PKB(Akt) pathway was critical in maintaining a differentiated state. The same signaling pathways involving in the phenotypic determination were observed in vascular SMCs. Thus, changes in the balance between the strengths of the PI 3-kinase/PKB(Akt) pathway and the ERK and p38MAPK pathways would determine phenotypes of visceral and vascular SMCs. [J. Cell Biol., Volume 145, Number 4, May 17, 1999 727-740 (Changes in the Balance of Phosphoinositide 3-Kinase/Protein Kinase B (Akt) and the Mitogen-activated Protein Kinases (ERK/p38MAPK) Determine a Phenotype of Visceral and Vascular Smooth Muscle Cells. Ken'ichiro Hayashi, Masanori Takahashi, Kazuhiro Kimura, Wataru Nishida, Hiroshi Saga, and Kenji Sobue].

Therefore, several differentiation-inducing agents activate PI3-kinase and the inhibition of the PI3K/p70S6K pathway blocks the process of differentiation in these cell lines (Postepy Hig Med Dosw 1999;53(2):305-13. Does the universal “signal transduction pathway of differentiation” exist? Comparison of different cell differentiation experimental models with differentiation of HL-60 cells in response to 1,25-dihydroxyvitamin D3, Marcinkowska E). Recent studies of bone growth and development indicate a critical role for P13-K signaling downstream of important factors of osteoclast and osteoblast differentiation and survival such as the CSF-1 receptor, RANK and alpha (V)B(3) integrin (Golden, et al, Bone 2004;34:3-12). Likewise, PI 3-kinase-mediated p70 S6 kinase activation has been shown to be critical to proliferation of human neural stem cells grown in culture (Ryu, et al J Neurosci Res 2003;72:352-62).

WO99/40783 and WO 00/18885 both teach that certain copper chelators can induce expansion of renewable stem cells from a variety of sources. These publications also teach that such expanded cells are CD38⁻.

Further, it has been reported that cellular PI 3-kinase activity was strongly enhanced after exposure to Cu⁺⁺ [Arch Biochem Biophys 2002 Jan. 15;397(2):232-9, copper ions strongly activate the phosphoinositide-3-kinase/Akt pathway independent of the generation of reactive oxygen species. Ostrakhovitch E A, Lordnejad M R, Schliess F, Sies H, Klotz L O].

However, the role of PI 3-kinase signalling in events associated with cell differentiation is as yet poorly understood. For example, Ptasznik et al (U.S. Pat. No. 6,413,773) teaches the induction of differentiation in undifferentiated human fetal cells using PI 3-kinase inhibitors. Other studies have demonstrated induction of differentiated monocytic phenotype in PMA-stimulated HL-60 cells with inhibition of PI 3-kinase (Park et al, Immunopharm Immunotox 2002;24:21-26).

In view of the findings described above, the present inventors have envisioned that by modulating the expression and/or the activity of PI 3-kinase, the expansion and differentiation of stem cells could be controlled. In particular, it was hypothesized that by reducing the expression and/or the activity of PI 3-kinase, using agents that downregulate the expression of PI 3-kinase or inhibit the activity thereof, expansion of renewable stem cells, devoid of differentiation, would be achievable.

Based on the above descriptions, it is clear that there is thus a widely recognized need for, and it would be highly advantageous to have, a method of propagating large numbers of stem cells in an ex-vivo setting. Methods enabling ex-vivo expansion of stem cell compartments yielding large numbers of these cell populations will therefore pioneer feasible stem cell therapies for human treatment, with a clear and direct impact on the treatment of an infinite number of pathologies and diseases. Some pathological and medically induced conditions are characterized by a low number of in-vivo self or transplanted renewable stem cells, in which conditions, it will be advantageous to have an agent which can induce stem cell expansion in-vivo.

### SUMMARY OF THE INVENTION

The present invention discloses the use of various modulators of PI 3-kinase for inducing ex-vivo and/or in-vivo expansion of stem cell populations, resulting, when applied, for example, to hematopoietic stem cells, in large numbers of undifferentiated CD34⁺/Lin⁻ (CD33, CD14, CD15, CD4, etc.), as well as CD34⁺/CD38⁻ cells, especially CD34⁺_{dim}/Lin⁻ cells.

This novel and versatile technology may be used for ex-vivo and in-vivo expansion of stem cells, of hematopoietic and other origins, maintaining their self-renewal potential for any in-vivo or ex-vivo application which requires large numbers of stem cell populations.

While reducing the present invention to practice, it was unexpectedly found that inhibitors of PI 3-kinase activity repress the process of differentiation of stem cells and stimulate and prolong the phase of active cell proliferation and expansion of the cells ex-vivo.

These unexpected effects were surprisingly obtained when the source of cells was CD34⁺ enriched hematopoietic cells (stem and early progenitor cells) and, most surprisingly, when the source of cells included the entire fraction of mononuclear blood cells (whole fraction of white blood cells, which includes stem, progenitor and committed cells).

Equally unexpected was the finding that primary hepatocyte cultures incubated with inhibitors of CD38, which is associated with PI 3-kinase signaling, revealed an increase in the proportion of cells producing α-fetoprotein, hence signaling the proliferation of early hepatocytes. Thus, it is expected that this newly discovered effect of modulators of PI 3-kinase activity and gene expression can be used for maximizing the ex-vivo expansion of various types of cells as is further detailed hereinunder.

According to one aspect of the present invention there is provided a method of ex vivo expanding and inhibiting differentiation of a population of stem cells, the method effected by: (a) providing the cells ex vivo with conditions for cell proliferation, and (b) ex vivo providing the cells with an effective concentration of a modulator of PI 3-kinase activity, the modulator selected capable of downregulating a PI 3-kinase activity or an expression of a gene encoding a PI 3-kinase; thereby ex vivo expanding and inhibiting differentiation of the population of stem cells.

According to another aspect of the present invention there is provided a method of transducing expanded, undifferentiated stem cells with an exogene, the method effected by (a) obtaining a population of stem cells; (b) expanding and inhibiting differentiation of the stem cells by: (i) providing the stem cells with conditions for cell proliferation and (ii) providing the stem cells with an effective concentration of a modulator of PI 3-kinase activity, the modulator selected capable of downregulating a PI 3-kinase activity or an expression of a gene encoding a PI 3-kinase; wherein steps (i) and (ii) are effected in vitro or ex vivo, thereby expanding and inhibiting differentiation of the stem cells; and (c) transducing the expanded, undifferentiated stem cells with the exogene.

According to further features in preferred embodiments of the invention described below the transducing is effected by a vector including the exogene.

According to yet further features in preferred embodiments of the invention described below the stem cells are early hematopoietic and/or hematopoietic progenitor cells.

According to a further aspect of the present invention there is provided a therapeutic ex vivo cultured stem cell population comprising undifferentiated hematopoietic cells expanded according to the methods of the present invention.

According to further features in preferred embodiments of the invention described below the cell population is provided in a culture medium comprising a modulator of PI 3-kinase activity, the modulator selected capable of downregulating a PI 3-kinase activity or an expression of a gene encoding a PI 3-kinase.

According to yet further features in preferred embodiments of the invention described below the cell population is isolated from said medium.

According to another aspect of the present invention there is provided a pharmaceutical composition comprising the cell population and a pharmaceutically acceptable carrier.

According to another aspect of the present invention there is provided a method of hematopoietic stem cells transplantation into a recipient, the method effected by: (a) obtaining a population of hematopoietic stem cells; (b) ex vivo expanding and inhibiting differentiation of the hematopoietic stem cells by: (i) ex vivo providing the hematopoietic stem cells with conditions for cell proliferation and (ii) providing the hematopoietic stem cells ex vivo with an effective concentration of a modulator of PI 3-kinase activity, said modulator selected capable of downregulating a PI 3-kinase activity or an expression of a gene encoding a PI 3-kinase, thereby expanding and inhibiting differentiation of the stem hematopoietic cells; and (c) transplanting the hematopoietic stem cells into the recipient.

According to yet another aspect of the present invention there is provided a method of adoptive immunotherapy comprising (a)obtaining progenitor hematopoietic cells from a patient, (b) ex vivo expanding and inhibiting differentiation of the hematopoietic cells by: (i) providing the progenitor hematopoietic cells ex vivo with conditions for cell proliferation and (ii) providing the progenitor hematopoietic cells with an effective concentration of a modulator of PI 3-kinase activity, the modulator selected capable of downregulating a PI 3-kinase activity or an expression of a gene encoding a PI 3-kinase, thereby expanding and inhibiting differentiation of the progenitor hematopoietic cells; and (c) transplanting the progenitor hematopoietic cells into a recipient.

According to another aspect of the present invention there is provided a method of mobilization of bone marrow stem cells into the peripheral blood of a donor for harvesting the cells, the method is effected by: (a) administering to the donor an effective concentration of a modulator of PI 3-kinase activity, said modulator selected capable of downregulating a PI 3-kinase activity or an expression of a gene encoding a PI 3-kinase, thereby expanding and inhibiting differentiation of a population of bone marrow stem cells and (b) harvesting the cells by leukopheresis.

According to yet another aspect of the present invention there is provided a method of inhibiting maturation/differentiation of erythroid precursor cells for treatment of a β-hemoglobinopathic patient comprising administering to the patient an effective concentration of a modulator of PI 3-kinase activity, said modulator selected capable of downregulating a PI 3-kinase activity or an expression of a gene encoding a PI 3-kinase, thereby expanding and inhibiting differentiation of a population of stem cells of the patient such that upon removal of the modulator of PI 3-kinse from said patient, the stem cells undergo accelerated maturation resulting in elevated fetal hemoglobin production, thereby ameliorating symptoms of β-hemoglobinopathy in the patient.

According to further features in preferred embodiments of the invention described below the method further comprising the step of administering a cytokine to the patient.

According to still another aspect of the present invention there is provided a method of preservation of undifferentiated stem cells comprising providing the undifferentiated stem cells with an effective concentration of a modulator of PI 3-kinase activity, the modulator selected capable of downregulating a PI 3-kinase activity or an expression of a gene encoding a PI 3-kinase of said undifferentiated stem cells. The providing is performed in at least one of the steps of harvesting, isolating and storage of the undifferentiated hematopoietic cells.

According to further features in preferred embodiments of the invention described below the method further comprising providing the cells with nutrients and cytokines.

According to further features in preferred embodiments of the invention described below the stem cells are early hematopoietic and/or hematopoietic progenitor cells.

According to yet further features in preferred embodiments of the invention described below the modulator selected capable of downregulating a PI 3-kinase activity or an expression of a gene encoding a PI 3-kinase is selected from the group consisting of (a) an inhibitor of PI 3-kinase catalytic activity, (b) an antisense polynucleotide capable of specifically hybridizing with an mRNA transcript encoding a PI 3-kinase, (c) a ribozyme which specifically cleaves PI 3-kinase transcripts, coding sequences and/or promoter elements, (d) an siRNA molecule capable of inducing degradation of PI 3-kinase transcripts; and (e) a DNAzyme which specifically cleaves PI 3-kinase transcripts or DNA.

According to still further features in preferred embodiments of the invention described below the inhibitor of PI 3-kinase activity is Wortmannin or LY294002.

According to further features in preferred embodiments of the invention described below the modulator capable of downregulating PI 3-kinase activity or expression of a gene encoding PI 3-kinase is an anti-PI 3-kinase antibody.

According to still further features in preferred embodiments of the invention described below the anti-PI 3-kinase antibody is ScFV or Fab.

According to further features in preferred embodiments of the invention described below the providing is effected by transiently expressing the antisense polynucleotide, the ribozyme, the siRNA molecule or the DNAzyme within a stem cell.

According to yet further features in preferred embodiments of the invention described below the providing is effected by (a) providing an expressible polynucleotide capable of expressing the antisense polynucleotide, the ribozyme, the siRNA molecule or the DNAzyme, and (b) stably integrating the expressible polynucleotide into a genome of a cell, thereby providing a modulator capable of downregulating a PI 3-kinase activity or PI 3-kinase gene expression.

According to still further features in preferred embodiments of the invention described below the inhibitor of PI 3-kinase activity is an expressible polynucleotide encoding an anti-PI 3-kinase ScFv or Fab.

According to further features in preferred embodiments of the invention described below providing the conditions for cell proliferation is effected by providing the cells with nutrients and cytokines, the cytokines being selected from the group consisting of early acting cytokines and late acting cytokines. According to yet further features in preferred embodiments of the invention described below the early acting cytokines are selected from the group consisting of stem cell factor, FLT3 ligand, interleukin-6, thrombopoietin and interleukin-3; and the late acting cytokines are selected from the group consisting of granulocyte colony stimulating factor, granulocyte/macrophage colony stimulating factor and erythropoietin.

According to further features in preferred embodiments of the invention described below the stem cells are derived from a source selected from the group consisting of hematopoietic cells, neural cells, oligodendrocyte cells, skin cells, hepatic cells, embryonal stem cells, muscle cells, bone cells, mesenchymal cells, pancreatic cells, chondrocytes and stroma cells.

According to yet further features in preferred embodiments of the invention described below the stem cells are derived from bone marrow or peripheral blood, or neonatal umbilical cord blood.

According to still further features in preferred embodiments of the invention described below the method of the present invention, further comprising the step of selecting a population of stem cells enriched for hematopoietic stem cells.

According to further features in preferred embodiments of the invention described below the selection is affected via CD34 or CD133.

According to further features in preferred embodiments of the invention described below the method further comprising the step of selecting a population of stem cells enriched for early hematopoietic stem/progenitor cells.

According to another aspect of the present invention there are provided stem cell collection bags, stem cell separation and stem cell washing buffers supplemented with an amount of a modulator of PI 3-kinase activity, said modulator selected capable of downregulating a PI 3-kinase activity or an expression of a gene encoding a PI 3-kinase, the amount sufficient to inhibit differentiation of a population of undifferentiated hematopoietic cells.

According to further features in preferred embodiments of the invention described below the modulator capable of downregulating PI 3-kinase activity or expression of a gene encoding PI 3-kinase is an inhibitor of PI 3-kinase activity or an anti-PI 3-kinase antibody.

According to yet further features in preferred embodiments of the invention described below the inhibitor of PI 3-kinase activity is Wortmannin or LY294002.

According to still further features in preferred embodiments of the invention described below the stem cell collection bags and buffers, further supplemented with nutrients and cytokines. The cytokines can be selected from the group consisting of early acting cytokines and late acting cytokines.

According to still further features in preferred embodiments of the invention described below the early acting cytokines are selected from the group consisting of stem cell factor, FLT3 ligand, interleukin-6, thrombopoietin and interleukin-3, and the late acting cytokines are selected from the group consisting of granulocyte colony stimulating factor, granulocyte/macrophage colony stimulating factor and erythropoietin.

According to another aspect of the present invention there is provided a assay for determining whether a modulator of PI 3-kinase activity is capable of inhibiting differentiation of cells, the assay comprising (a) culturing a population of cells capable of differentiating, in the presence or absence of the modulator of PI 3-kinase activity and (b) assessing changes in differentiation of the cells. An increase in differentiation as compared to untreated cells indicates a modulator of PI 3-kinase activity incapable of inhibiting differentiation, and a lack of or decrease in differentiation as compared to untreated cells, indicates a modulator of PI 3-kinase activity capable of inhibiting differentiation.

According to further features in preferred embodiments of the invention described below the cells capable of differentiating are stem or progenitor cells, or substantially undifferentiated cells of a cell line.

According to further features in preferred embodiments of the invention described below the stem or progenitor cells are early hematopoietic and/or hematopoietic progenitor cells.

According to still further features in preferred embodiments of the invention described below the assay further comprising providing the cells with nutrients and cytokines.

The cytokines can be selected from the group consisting of early acting cytokines and late acting cytokines.

According to still further features in preferred embodiments of the invention described below the early acting cytokines are selected from the group consisting of stem cell factor, FLT3 ligand, interleukin-6, thrombopoietin and interleukin-3, and the late acting cytokines are selected from the group consisting of granulocyte colony stimulating factor, granulocyte/macrophage colony stimulating factor and erythropoietin.

According to yet further features in preferred embodiments of the invention described below the stem cells are derived from a source selected from the group consisting of hematopoietic cells, neural cells, oligodendrocyte cells, skin cells, hepatic cells, embryonal stem cells, muscle cells, bone cells, mesenchymal cells, pancreatic cells, chondrocytes and stroma cells.

According to still further features in preferred embodiments of the invention described below, assessing changes in differentiation is effected via differentiation markers.

According to further features in preferred embodiments of the invention described below the differentiation markers are selected from the group consisting of CD133, CD34, CD38, CD33, CD14, CD15, CD3, CD61 and CD19.

According to another aspect of the present invention there is provided a method of ex vivo expanding and inhibiting differentiation of a population of stem cells, the method comprising: (a) providing the cells ex vivo with conditions for cell proliferation and (b) ex vivo reducing a capacity of said stem cells in responding to signaling pathways involving a PI 3-kinase activity; thereby ex vivo expanding and inhibiting differentiation of the population of stem cells.

The present invention successfully addresses the shortcomings of the presently known configurations by providing a method of propagating cells, yet delaying their differentiation by interference with CD38 or PI 3-kinase expression, activity, and/or PI 3-kinase signaling.

The present invention further successfully addresses the shortcomings of the presently known configurations by enabling, for the first time, expansion of renewable stem cells in the presence of committed cells, so as to obtain an expanded population of renewable stem cells, albeit their origin from a mixed population of cells, in which they constitute a fraction of a percent.

Additional features and advantages of the methods cell preparations and articles of manufacture according to the present invention will become apparent to the skilled artisan by reading the following descriptions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a FACS analysis plot showing control cell surface marker expression with liberal expression of CD34, CD38 and lineage-related antigens.

FIG. 1B is a FACS analysis plot showing RAR antagonist (10⁻⁵ M) treated culture cell surface marker expression with a similar level of expression of the CD34 antigen, but an almost complete abrogation of the CD38 and lineage-related antigen expression, as compared to controls.

FIG. 1C is a FACS analysis plot showing RAR antagonist (10⁻⁶ M) treated culture cell surface marker expression with a similar level of expression of the CD34 antigen, but an almost complete abrogation of the CD38 and lineage-related antigen expression, as compared to controls.

FIG. 2A is a graph of data collected by FACS analysis showing comparable CD34⁺ cell expansion levels in control and RAR antagonist treated cultures.

FIG. 2B is a graph of data collected by FACS analysis showing markedly enhanced CD34⁺CD38⁻ cell expansion levels in response to RAR antagonist treatment, at either the 10⁻⁵ or 10⁻⁷ M concentrations, as compared to controls.

FIG. 2C is a graph of data collected by FACS analysis showing markedly enhanced CD34⁺Lin⁻ cell expansion levels in response to RAR antagonist treatment, at either the 10⁻⁵ or 10⁻⁷ M concentrations, as compared to controls.

FIG. 3A is a graph of data collected by FACS analysis revealing comparable CD34⁺ surface expression up to 2 weeks post seeding of control and treated cultures. Cultures were treated with an RAR antagonist, 10⁻⁵ M and 10⁻⁷ M [or 41 μg/liter to 0.41 μg/liter] and a combination of 4 cytokines (IL-6, TPO, FLT3 and SCF), and were subjected to an additional positive selection step prior to FACS analysis. A marked increase in expression is seen, however, 9 and 11 weeks post seeding in cultures treated with RAR antagonists, as compared to controls.

FIG. 3B is a graph of data collected by FACS analysis showing comparable CD34⁺CD38⁻ surface expression up to 2 weeks post seeding of control and RAR antagonist and cytokine treated cultures, (as treated in 3A), in samples subjected to an additional positive selection step. A marked increase in expression is seen 9 and 11 weeks post seeding in RAR antagonist treated cultures, as compared to controls.

FIG. 3C is a graph of data collected by FACS analysis showing enhanced CD34⁺Lin⁻ surface expression by 2 weeks post seeding of RAR antagonist treated cultures, (as treated in 3A), as compared to controls, in samples subjected to an additional positive selection step. A markedly increased expression is seen in the groups treated with RAR antagonist by 9 and 11 weeks post seeding.

FIG. 4 is a graph of data collected by FACS analysis and LTC-CFU ability showing high levels of CD34⁺ cell proliferation and long-term colony forming unit ability in ex-vivo cultures treated with 10⁻⁷ M of the RAR antagonist and a combination of the 4 cytokines, as above, up to almost 12 weeks post seeding. At 10 weeks and 11 weeks (CFUs and CD34 cells, respectively), these populations begin to decline.

FIG. 5A is a FACS analysis plot of the negative control showing no background staining.

FIG. 5B is a FACS analysis plot of the positive control of reselected cell cultures showing ample CD34⁺ cell surface staining.

FIG. 5C is a FACS analysis plot of the RAR antagonist treated cultures 2 weeks post reselection showing a marked leftward shift in profile, consistent with a less differentiated state.

FIG. 5D is a FACS analysis plot of the RAR antagonist treated cultures (10⁻⁷) 11 weeks post reselection showing ample CD34⁺ cell surface staining, and a profile consistent with a more differentiated state.

FIG. 5E is a FACS analysis plot of the RAR antagonist treated cultures (10⁻⁵) 11 weeks post reselection showing a marked leftward shift in profile, consistent with a less differentiated state.

FIG. 6A is a graph of colony forming unit data showing that both long-term cultures pulsed for the first 3 weeks with the antagonists or cultures administered RAR antagonists continuously reveal a 5-fold increase in CFU content as compared to control values.

FIG. 6B is a graph of cell enumeration data showing that long-term cultures either pulsed for the first 3 weeks with antagonists, or administered RAR antagonists continuously, reveal a 5-fold increase in CFU content as compared to control values.

FIG. 7 is a graph of mixed colony forming unit data showing that both long-term cultures pulsed for the first 3 weeks with the antagonists or cultures administered RAR antagonists continuously reveal a dramatic increase in CFU content as compared to control values, with pulse-treatment yielding the highest CFU values.

FIG. 8A is a photomicrograph of three weeks old primary hepatocyte cultures isolated from mice. Hepatocytes were probed for expression of α-fetoprotein (AFP) and counterstained with hematoxylin. Moderate AFP staining is evident (red-brown precipitate).

FIG. 8B is a photomicrograph of three week old primary hepatocyte cultures isolated from mice. Hepatocytes were incubated in the presence of 10⁻⁵ M retinoic acid receptor antagonist (AGN 194310) and were similarly probed for AFP expression and counterstained with hematoxylin. AGN 194310-treated hepatocytes revealed a marked increase in AFP expression, as compared to controls.

FIG. 9A is a photomicrograph of giemsa stained, three week old, primary murine hepatocyte cultures revealing cell morphology. Few oval cells were noted in this sample (thick arrow), in contrast to numerous hepatocytes with typical morphology (narrow arrow)

FIG. 9B is a photomicrograph of giemsa stained, primary hepatocyte cultures incubated in the presence of 10⁻⁵ M retinoic acid receptor antagonist (AGN 194310). Antagonist treated cells showed a marked increase in oval cell population (arrow).

FIG. 9C is a photomicrograph of giemsa stained, primary hepatocyte cultures incubated in the presence of 10⁻⁵ M retinoic acid receptor antagonist (AGN 194310) followed by trypsinization and replating, at a ratio of 1:2, in a culture medium devoid of cytokines. These cultures similarly revealed characteristic hepatocyte morphology

FIG. 10A is a photomicrograph of three weeks old primary hepatocyte cultures isolated from mice, and supplemented with EGF (20 ng/ml) and HGF (20 ng/ml). Hepatocytes were treated with RAR antagonist AGN 194310 at 10⁻ M to 10⁻⁷ M, probed for expression of albumin and counterstained with hematoxylin. There is no appreciable background staining. Indicated that the cells expanded in cultures supplemented with the antagonist are hepatocytes by nature.

FIG. 10B is a photomicrograph of three weeks old primary hepatocyte control cultures isolated from mice, similarly supplemented with EGF and HGF and probed for albumin expression. Negligible background staining is evident here as well.

FIG. 10C is a photomicrograph of three weeks old primary hepatocyte RAR antagonist treated cultures isolated from mice, similarly supplemented with EGF and HGF and probed for α-fetoprotein expression. Significant strong AFP staining is evident (red-brown precipitate), indicating expansion of progenitor cells.

FIG. 10D is a photomicrograph of three weeks old primary hepatocyte control cultures isolated from mice, similarly supplemented with EGF and HGF and probed for α-fetoprotein expression. Negligible staining is evident indicating a more differentiated cellular phenotype. All figures were photographed at 10×/0.3 magnification.

FIG. 11A is a photomicrograph of first passage heaptocyte control cultures isolated from mice and supplemented with EGF and HGF, split 1:2 following 2 weeks in culture and cultured for an additional week prior to probing for albumin expression, as above. Numerous typical hepatocytes (small arrow) are evident.

FIG. 11B is a photomicrograph of first passage RAR antagonist AGN 194310 (10⁻⁵-10⁻⁷ M) treated heaptocyte cultures isolated from mice cultured as in A and probed for albumin expression. Typical hepatocyte morphology (small arrow) is evident in this frame as well.

FIG. 11C is a photomicrograph of first passage RAR antagonist treated hepatocyte cultures, cultured and probed as in B. Numerous characteristic oval cells are evident (large arrow) in the field. Magnification—20×/0.5.

FIG. 11D is a photomicrograph is a lower magnification of FIG. 11C, revealing numerous islets of oval cells in the RAR antagonist treated cultures, consistent with a less-differentiated phenotype.

FIG. 11E is a photomicrograph of second passage heaptocyte control cultures isolated from mice and supplemented with EGF and HGF, split 1:2 following 2 weeks in culture, cultured for an additional week prior to 1:4 split, and following a final additional 4 day culture, probing for albumin expression, as above. Few hepatocytes are evident.

FIG. 11F is a photomicrograph of similarly isolated and cultured second passage heaptocyte cultures treated with RAR antagonist AGN 194310 (10⁻⁵ M to 10⁻⁷ M). Significantly greater numbers of hepatocytes are evident in the cultures as compared to controls. Magnification—20×/0.5.

FIG. 12A is a plot presenting the FACS analysis of cultures treated with cytokines only (control), RAR antagonist AGN 194310 (10⁻⁷ M) and a combination of RAR antagonist (10⁻⁷ M) and RXR antagonist, 3 weeks post reselection. A marked leftward shift in profile of the combined, RAR and RXR antagonists, treatment, consistent with a less differentiated state, as compared with the untreated control and the RAR antagonist treatment is demonstrated.

FIG. 12B is a plot presenting a FACS analysis of cultures treated with cytokines only (control), RAR antagonist AGN 194310 (10⁻⁷ M), RXR antagonist LGN 100754 (10⁻⁷ M) and a combination of RAR and RXR antagonists (10⁻⁷ M), 5 weeks post reselection. A marked leftward shift in profile of the combined, RAR and RXR antagonists, treatment, consistent with a less differentiated state, as compared with the RAR antagonist treatment is demonstrated.

FIG. 13A is a bar graph presenting the data obtained by FACS analysis of cultures treated with a RAR antagonist AGN 194310, a RXR antagonist LGN 100754 and a combination thereof. Comparable CD34⁺ surface expression levels determined 3 and 5 weeks post seeding are evident. A marked increase in expression in cultures treated with a combination of the RAR and RXR antagonists, as compared with the untreated (cytokines only) control, the RAR antagonist and RXR antagonist treatments is demonstrated.

FIG. 13B is a bar graph presenting the data obtained by FACS analysis of cultures treated with an RAR antagonist AGN 194310, an RXR antagonist LGN 100754 and a combination thereof. Comparable CD34⁺/38⁻ surface expression levels determined 3 and 5 weeks post seeding are evident. A marked increase in expression in cultures treated with the combination of RAR and RXR antagonists, as compared with the untreated control (cytokines only), the RAR antagonist and the RXR antagonist treatments is demonstrated.

FIG. 13C is a bar graph presenting the data obtained by FACS analysis of cultures treated with an RAR antagonist AGN 194310, an RXR antagonist LGN 100754 and a combination thereof. Comparable CD34⁺/Lin⁻ surface expression levels determined 3 and 5 weeks post seeding are evident. A marked increase in expression in cultures treated with the RAR and RXR antagonists combination, as compared with the untreated control (cytokines only), the RAR antagonist and the RXR antagonist treatments is demonstrated.

FIG. 13D is a bar graph presenting the total cell density of cultures treated with an RAR antagonist AGN 194310, an RXR antagonist LGN 100754 and a combination thereof. Comparable number of cells determined 3 and 5 weeks post seeding is evident. A significant increase of cell density in cultures treated with RAR+RXR antagonist 5 weeks post seeding, as compared with the untreated control (cytokines only), the RAR antagonist and RXR antagonist treatments is demonstrated.

FIG. 13E is a bar graph presenting the colony-forming unit (CFU) data of cultures treated with an RAR antagonist AGN 194310, an RXR antagonist LGN 100754 and a combination thereof. Comparable CFU levels determined 3 and 5 weeks post seeding are evident. A marked increase in CFU in cultures treated with the RAR and RXR combination, as compared with the untreated control (cytokines only), the RAR antagonist and the RXR antagonist treatments is demonstrated.

FIG. 14 is a bar graph presenting the density of CD34+ cells enumerated in 3 weeks culture. The cell culture was supplemented with SCF, TPO, FLt3, IL-6 and IL-3 cytokines, with or without nicotinamide at 1 mM and 5 mM concentrations. A marked increase in CD34+ cells density in the nicotinamide treated cultures is demonstrated.

FIG. 15 is a bar graph presenting the data obtained by FACS analysis of CD34+/CD38− cells in 3 weeks culture. The cell culture was supplemented with SCF, TPO, FLt3, IL-6 and IL-3 cytokines, with or without nicotinamide at 1 mM and 5 mM concentrations. A marked increase in CD34+/CD38− cell density in the nicotinamide treated cultures is demonstrated.

FIG. 16 is a bar graph presenting the data obtained by FACS analysis of CD34+/Lin− cells in 3 weeks culture. The cell culture was supplemented with SCF, TPO, FLt3, IL-6 and IL-3 cytokines, with or without nicotinamide at 1 mM and 5 mM concentrations. A marked increase in CD34+/Lin− cell density in the nicotinamide treated cultures is demonstrated.

FIG. 17 is a bar graph presenting the data obtained by FACS analysis of CD34+/(HLA-DR38)− cells in 3 weeks culture. The cell culture was supplemented with SCF, TPO, FLt3, IL-6 and IL-3 cytokines, with or without nicotinamide at 1 mM and 5 mM concentrations. A marked increase in CD34+/(HLA-DR38)− cell density in the nicotinamide treated cultures is demonstrated.

FIG. 18a is a dot plot presenting a FACS analysis of re-selected CD34+ cells from a 3 weeks culture treated with cytokines, with or without 5 mM nicotinamide. The CD34+/CD38− cells are shown in the upper left part of the plot, demonstrating a marked increase of CD34+/CD38− cells in the nicotinamide treated culture.

FIG. 18b is a dot plot presenting a FACS analysis of re-selected CD34+ cells from a 3 weeks culture treated with cytokines, with or without 5 mM nicotinamide, 3 weeks post reselection. The CD34+/Lin− cells are shown in the upper left part of the plot, demonstrating a marked increase of CD34+/Lin− cells in the nicotinamide treated culture.

FIG. 18c is a dot plot presenting a FACS analysis of re-selected CD34+ cells from a 3 weeks culture treated with cytokines, with or without 5 mM nicotinamide, 3 weeks post reselection. The CD34+/(HLA-DR38)− cells are shown in the upper left part of the plot, demonstrating a marked increase of CD34+/+/(HLA-DR38)− cells in the nicotinamide treated culture.

FIG. 19 shows the short-term effect of TEPA on the clonlogenic potential of CD₃₄ cells. Cord blood-derived CD₃₄ cells were plated in liquid culture, at 3×10⁴ cell/ml, in the presence of low dose cytokines: FLT3—5 ng/ml, SCF—10 ng/ml, IL-6—10 ng/ml, with or without different concentrations of TEPA. On day 7, aliquots of 0.1 ml were assayed for colony forming cells by cloning the cells in semi-solid medium and scoring colonies after 14 days. Results of two independent experiments are presented.

FIG. 20 shows the short-term effect of TEPA on total and CD₃₄ cells. Cord blood-derived CD34 cells were plated in liquid culture in the presence of FL—5 ng/ml, SCF—10 ng/ml, IL-6—10 ng/ml, with or without of TEPA (20 μM). On day 7, the wells were demi-depopulated by removal of one half the culture volume and replacing it with fresh medium and IL-3 (20 ng/ml). On day 14, the percentage of CD₃₄ cells (right) and the total cell number (left) multiplied by the dilution factor were determined.

FIGS. 21a-b show the long-term effect of TEPA on cell number and clonogenic potential of CD₃₄ cells. Cord blood-derived CD₃₄ cells were plated in liquid culture, at 3×10⁴ cells/ml, in the presence of high dose cytokines: FL—50 ng/ml, SCF—50 ng/ml, IL-6—50 ng/ml, IL-3—20 ng/ml, G-CSF—10 ng/ml, EPO—1 U/ml, with or without TEPA (20 μM). On day 4, the cultures were diluted 1:10 with 0.9 ml fresh medium supplemented with cytokines and TEPA. On day 7, 14 and 21, the cultures were demi-depopulated by removal of one half the culture volume and replacing it with fresh medium, cytokines and TEPA, as indicated. Cells of the harvested medium were count and aliquots equivalent to 1×10³ initiating cells were cloned in semi-solid medium. The numbers of cells (a) in the liquid culture and of colonies (b) in the semi-solid culture, multiplied by the dilution factors, are represented. * denotes small colonies and cell clusters.

FIGS. 22a-b show the long-term effect of TEPA on CD₃₄ cells cultured with early cytokines. Cord blood-derived CD₃₄ cells were plated in liquid culture in the presence of: FL—50 ng/ml, SCF—50 ng/ml and thrombopoietin (TPO)—20 ng/ml, with or without TEPA (10 μM). At weekly intervals, the cultures were demi-depopulated by removal of one half the culture volume and replacing it with fresh medium, cytokines and TEPA, as indicated. Cells of the harvested medium were count and aliquots equivalent to 1×10³ initiating cells were cloned in semi-solid medium. The numbers of colonies (a) and numbers of cells (b) in the liquid culture in the semi-solid culture, multiplied by the dilution factors, are represented. * denotes that no colonies developed.

FIG. 23 shows the effect of TEPA on development of erythroid precursors. Peripheral blood mononuclear cells, obtained from an adult normal donor, were cultured in the erythroid two-phase liquid culture system (23-25). The second phase of the culture was supplemented either without or with 10 μM of TEPA. Cultures were analyze for total cells and hemoglobin-containing [benzidine positive (B⁺)] cells after 14 days.

FIGS. 24a-d show the effect of TEPA on cell maturation. Morphology of cells in long-term (7 weeks) cultures in the absence (24a and 24c) and presence (24b and 24d) of TEPA is shown. Cytospin prepared slides were stained with May-Grunwald Giemsa. Magnifications: 6a and 6b×600; 6c and 6d×1485.

FIG. 25 shows the effect of transition metal chelators on cell number and clonogenic of CD₃₄ cells initiated cultures. Cord blood-derived CD₃₄ cells were plated in liquid cultures in the presence of FL—20 ng/ml, SCF—20 ng/ml, IL-3—20 ng/ml, IL-6—20 ng/ml, and either TEPA—10 μM, captopril (CAP)—10 μM or Penicillamine (PEN)—10 μM, as indicated. On day 7, cells were counted and culture aliquots equivalent to 1×10³ initiating cells were plated in semi-solid medium. The bars present the total cell number (×10³/ml) on day 7 and the number of colonies per plate 14 days following cloning.

FIGS. 26a-b show the effect of Copper on the clonogenic potential and total cell number of CD₃₄ cells. Cord blood-derived CD₃₄ cells were plated in liquid cultures in the presence of cytokines: FL—10 ng/ml, SCF—10 ng/ml, IL-3—10 ng/ml, IL-6—10 ng/ml. Cultures were supplemented with Copper-sulfate—5 μM and TEPA—20 μM, as indicated. On day 7, cells were counted (b) and aliquots equivalent to 1×10³ initiating cells were plated in semi-solid medium. Colonies were scored after 14 days (a).

FIG. 27 shows the effect of ions on the clonogenic potential of cultured CD₃₄ cells. Cord blood-derived CD₃₄ cells were plated in liquid cultures in the presence of FL—10 ng/ml, SCF—10 ng/ml, IL-3—10 ng/ml, IL-6—10 ng/ml, and either with or without TEPA—10 μM. The cultures were supplemented with Copper-sulfate—5 mM, sodium selenite—5 mM or iron-saturated transferrin 0.3 mg/ml, as indicated. On day 7, culture aliquots equivalent to 1×10³ initiating cells were plated in semi-solid medium. Colonies were scored after 14 days.

FIG. 28 shows the effect of Zinc on the proliferative potential of CD₃₄ cells. Cord blood-derived CD₃₄ cells were plated in liquid cultures in the presence of FL—10 ng/ml, SCF—10 ng/ml, IL-3—10 ng/ml, IL-6—10 ng/ml, and either TEPA—10 μM or Zinc-sulfate—5 mM or both. On day 7, aliquots equivalent to 1×10³ initiating cells were plated in semi-solid medium. Colonies were scored after 14 days.

FIGS. 29a-c show the effect of TEPA on long-term CD₃₄ cultures. Cultures were initiated with 10⁴ cord blood-derived CD₃₄ cells by plating purified cells in liquid medium in the presence of SCF, FLT3 and IL-6 (50 ng/ml each) and IL-3 (20 ng/ml) with or without TEPA (10 μM). At weekly intervals, the cultures were demi-depopulated by removal of half the cells followed by addition of fresh medium, cytokines and TEPA. At the indicated weeks, cells were counted and assayed for colony forming cells (CFUc) by cloning in semi-solid medium. CFUc frequency was calculated as number of CFUc per number of cells. Cloning of purified CD₃₄ cells on day 1 yielded 2.5×10³ CFUc per 10⁴ initiating cells. * denotes that no colonies developed.

FIGS. 30-32 show the effect of TEPA on cell proliferation, CFUc and CFUc frequency in the presence of different combination of early cytokines. Cord blood-derived CD₃₄ cells were cultured as detailed in FIGS. 11a-c in liquid medium in the presence of SCF, FLT3 and IL-6 (SCF, FLT, I1-6), each at 50 ng/ml, with or without TEPA (10 μM). In addition, cultures were supplemented with either IL-3 (20 ng/ml), TPO (50 ng/ml) or both, as indicated. At weekly intervals, the cultures were demi-depopulated and supplemented with fresh medium, cytokines and TEPA. At the indicated weeks, the cells were counted (FIG. 30), assayed for CFUc (FIG. 31) and the CFUc frequency calculated (FIG. 32). * denotes that no colonies developed.

FIG. 33 shows the effect of G-CSF and GM-CSF on CFUc frequency of control and TEPA-supplemented CD₃₄ cultures. Cord blood-derived CD₃₄ cells were cultured as detailed in FIGS. 11a-c. After one week, half of the control and TEPA cultures were supplemented with the late-acting cytokines G-CSF and GM-CSF (10 ng/ml each). At weekly intervals, the cultures were demi-depopulated and supplemented with fresh medium, cytokines and TEPA. At weeks 3, 4 and 5, cells were counted, assayed for CFUc and CFUc frequency calculated.

FIGS. 34-35 show the effect of partial or complete medium+TEPA change on long-term cell proliferation and CFUc production. Cord blood-derived CD₃₄ cells were cultured as detailed in FIGS. 11a-c. At weekly intervals, the cultures were demi-depopulated and supplemented with fresh medium, cytokines and TEPA. At weekly intervals, half of the culture content (cells and supernatant) was removed and replaced by fresh medium, cytokines with or without TEPA (partial change). Alternatively, the whole content of the culture was harvested, centrifuged, the supernatant and half of the cells discarded and the remaining cells recultured in fresh medium, cytokines with or without TEPA (complete change). At the indicated weeks the number of cells (FIG. 34) and CFUc (FIG. 35) were determined.

FIG. 36 show the effect of TEPA on CD₃₄ cell expansion. Cord blood-derived CD₃₄ cells were cultured as detailed in FIGS. 29a-c. At weeks 1, 2 and 3, CD₃₄⁺ cells were enumerated by flow cytometry. * denotes that no colonies developed.

FIG. 37 shows the effect of delayed addition of TEPA on CFUc frequency. Cord blood-derived CD₃₄ cells were cultured as detailed in FIGS. 29a-c. TEPA (10 μM) was added at the initiation of the cultures (day 1) or 6 days later. At weekly intervals, the cultures were demi-depopulated and supplemented with fresh medium, cytokines and TEPA. At weeks 3, 4 and 5, cells were counted, assayed for CFUc and the CFUc frequency was calculated.

FIG. 38 shows the effect of short-term preincubation with a single cytokine on long-term CFUc production. Cord blood-derived CD₃₄ cells were cultured as detailed in FIGS. 11a-c. Cultures were supplemented on day 1 with or without TEPA (10 μM) and with SCF, FLT3, IL-6, (50 ng/ml each) and IL-3 (20 ng/ml). Alternatively, cultures were supplemented on day 1 with TEPA (10 μM) and FLT3 (50 ng/ml) as a single cytokine. SCF, IL-6 (50 ng/ml each) and IL-3 (20 ng/ml) were added to these cultures at day 2. At weekly intervals, the cultures were demi-depopulated and supplemented with fresh medium, cytokines and TEPA. At the indicated weeks cells were assayed for CFUc.

FIGS. 39a-b show the effect of polyamine chelating agents on CD₃₄ cell cultures. Cord blood-derived CD₃₄ cells were cultured as detailed in FIGS. 29a-c. The polyamine chelating agents tetraethylenepentamine (TEPA), penta-ethylenehexamine (PEHA), ethylenediamine (EDA) or triethylene-tetramine (TETA) were added, at different concentrations. At weekly intervals, the cultures were demi-depopulated and supplemented with fresh medium, cytokines and chelators. At weeks 3, 4, 6 and 7, cells were counted and assayed for CFUc. The results presented are for concentrations with optimal activity: TEPA—40 μM, PEHA—40 μM, EDA—20 μM and TETA—20 μM.

FIGS. 40a-b show the effect of transition metal chelating agents on CD₃₄ cell cultures. Cord blood-derived CD₃₄ cells were cultured as detailed in FIGS. 29a-c. The chelators Captopril (CAP), Penicilamine (PEN) and TEPA were added, at different concentrations. At weekly intervals, the cultures were demi-depopulated and supplemented with fresh medium, cytokines and chelators. At the weeks 4, 5 and 7, cells were counted and assayed for CFUc. The results presented are for concentrations with optimal activity: TEPA—10 μM, PEN—5 μM and CAP—40 μM.

FIGS. 41a-b show the effect of Zinc on CD₃₄ cell cultures. Cord blood-derived CD₃₄ cells were cultured as detailed in FIGS. 29a-c. Zinc (Zn) was added, at different concentrations, on day 1. At weekly intervals, the cultures were demi-depopulated and supplemented with fresh medium, cytokines and Zn. At the weeks 4, 5 and 7, cells were counted and assayed for CFUc.

FIG. 42 shows the effect of TEPA on peripheral blood derived CD₃₄ cell cultures. Peripheral blood-derived CD₃₄ cells were cultured as detailed in FIGS. 29a-c. Cultures were supplemented with or without TEPA. At weekly intervals, the cultures were demi-depopulated and supplemented with fresh medium and TEPA. At weeks 1 and 4, and, cells were assayed for CFUc. * denotes that no colonies developed.

FIGS. 43a-b show the effect of Copper-chelating peptides on CD₃₄⁺ cell cultures. Cultures were initiated with 10⁴ cord blood-derived CD₃₄⁺ cells by plating purified cells in liquid medium in the presence of SCF, FLT3 and IL-6 (50 ng/ml each) and the Copper-binding peptides, Gly-Gly-His (GGH) or Gly-His-Lys (GHL) (10 μM each), or the late-acting cytokines granulocyte-CSF (G-CSF) and granulocyte macrophage-CSF (GM-CSF) (10 ng/ml each). At weekly intervals, the cultures were demi-depopulated and supplemented with fresh medium, cytokines and the peptides. After 7 weeks, cells were counted (FIG. 43a) and assayed for colony forming cells in culture (CFUc, FIG. 43b).

FIG. 44 shows the chemical structure of transition metal chelators used in an assay according to the present invention, which can be used to determine the potential of any chelator to arrest or induce cell differentiation.

FIGS. 45a-f show photographs of hepatocytes cultures that were ex-vivo expanded with (45a-d) or without (45e-f) TEPA for five weeks.

FIG. 46 illustrates the effect of inhibition of PI 3-kinase on hematopoietic stem cell differentiation. The graphs show a representative FACS analysis dot plot of early CD34+ cell subsets, re-purified from 2-week control and Ly294002-treated cultures, using a MiniMACS CD34 progenitor cell isolation kit (Miltenyi). The purified cells were stained for markers CD34/CD38 and CD34/Lin (CD38, CD33, CD14, CD15, CD3, CD61, CD19), using PE and FITC labeled antibodies, as described. The percentages of CD34+CD38− and CD34+Lin− cells are shown in the upper left of the plots.

FIGS. 47a and 47b show the effect of inhibition of PI 3-kinase on hematopoietic stem cell morphology in culture. Morphology of cells in 3 weeks cultures in the absence (control, cytokines, 47b) and presence (LY294002 47a) of the PI 3-kinase inhibitor (5 μM/L) is shown. Cytospin-prepared slides were stained with May-Grunwald/Giemsa. Note the rounded appearance and lack of granules typical of stem cells in the LY294002 treated cultures (FIG. 47a), compared with the macrophage-like appearance of lamellipodia and numerous inclusions in the control cultures (FIG. 47b). Scale bar equals 500 μm.

### DESCRIPTION OF THE PREFERRED EMBODIMENTS

The present invention is of methods of expanding a population of stem cells, while at the same time, substantially inhibiting differentiation of the cells ex-vivo and/or in-vivo. In one embodiment, the invention facilitates the efficient use as a therapeutic ex-vivo cultured cell preparation, which includes an expanded, large population of renewable stem cells, in which differentiation was inhibited while cell expansion was propagated. Specifically in this respect, the present invention can be used to provide ex-vivo expanded populations of stem cells, which can be used for applications in hematopoietic cell transplantations, and in generation of stem cells suitable for genetic manipulations, which may be used for cellular gene therapy. Additional applications may include, but are not limited to, adoptive immunotherapy, treatments for multiple diseases, such as, for example, β-hemoglobinopathia, implantation of stem cells in an in vivo cis-differentiation and trans-differentiation settings, and ex vivo tissue engineering in cis-differentiation and trans-differentiation settings. The present invention further relates to expanded stem cell preparations and to articles-of-manufacture for preparing same.

The present invention discloses the use of various molecules (also referred to herein as agents and/or modulators), for interfering with PI 3-kinase expression and/or activity, thereby inducing ex-vivo expansion of stem cell populations, resulting, when applied, for example, to hematopoietic stem cells, in large numbers of undifferentiated CD34⁺/Lin⁻ (CD33, CD14, CD15, CD4, etc.), as well as CD34⁺/CD38⁻ cells, and CD34⁺_{dim}/Lin⁻ cells. This novel and versatile technology may be used for ex-vivo and/or in-vivo expansion of stem cells, of hematopoietic and other origins, maintaining their self-renewal potential for any in-vivo or ex-vivo application which requires a large population of stem cells.

While reducing the present invention to practice, it was unexpectedly found that molecules that are capable of interfering with PI 3-kinase expression and/or activity, repress the process of differentiation of stem cells and stimulates and prolong the phase of active cell proliferation and expansion ex-vivo. In similar experiments with inhibitors of CD38 expression, it was found that following about 16-18 weeks of expansion, the cells begin to differentiate; hence, the effect of these molecules is reversible. In other words, treating the cells ex-vivo as herein described does not result in the cells transforming into a cell line.

This unexpected effect was surprisingly obtained when the source of cells was CD34⁺ enriched hematopoietic cells (stem and early progenitor cells) and, most surprisingly, also when the source of cells included the entire fraction of mononuclear blood cells (whole fraction of white blood cells, which includes stem, progenitor and committed cells) were used. As is described in the Background section, presently there is no disclosed technology by which to expand non-enriched stem cells.

Furthermore primary hepatocyte cultures incubated with agents inhibiting CD38 expression, revealed an increase in the proportion of cells producing α-fetoprotein, hence inducing the proliferation of early hepatocyte populations. Antagonist-treated hepatocyte cultures grown without cytokines persisted for at least 3 weeks in culture, a finding in stark contrast to previous data indicating an almost impossibility in growing primary hepatocytes for extended periods of time in culture, especially in the absence of cytokines (Wick M, et al. ALTEX. 1997; 14(2): 51-56; Hino H, et al. Biochem Biophys Res Commun. 1999 Mar. 5;256(1): 184-91; and Tateno C, and Yoshizato K. Am J Pathol. 1996; 148(2): 383-92). Supplementation with growth factors alone was insufficient to stimulate hepatocyte proliferation, only CD38 inhibitor treatment of hepatocyte cultures resulted in the proliferation of early hepatocyte populations and in their persistence in culture, evident even following first and second passages.

This newly discovered effect of the molecules suitable in context of the present invention was used for maximizing the ex-vivo expansion of various types of cells as is further detailed hereinunder and exemplified in the Examples section that follows.

The principles and operation of the present invention may be better understood with reference to the drawings and accompanying descriptions and examples.

Before explaining at least one embodiment of the invention in detail, it is to be understood that the invention is not limited in its application to the details of construction and the arrangement of the components set forth in the following description or illustrated in the Examples section. The invention is capable of other embodiments or of being practiced or carried out in various ways. Also, it is to be understood that the phraseology and terminology employed herein is for the purpose of description and should not be regarded as limiting.

CD38 is a member of an emerging family of cytosolic and membrane-bound enzymes whose substrate is nicotinamide adenine dinucleotide (NAD). Two of the metabolites produced by CD38, cADPR and NAADP, have been shown to induce the release of intracellular calcium in cells isolated from tissues of plants, invertebrates and mammals, suggesting that these metabolites may be global regulators of calcium responses (Lee et al., 1999 Biol. Chem. 380;785-793).

Recently, it has been reported that granulocytic differentiation of the human committed cell line HL-60 cells can be induced by retinoic acid and is accompanied by a massive expression of CD38. Concomitant with CD38 expression was the accumulation of cADPR, and both time courses preceded the onset of differentiation, suggesting a causal role for CD38. Consistently, treatment of HL-60 cells with a permanent inhibitor of CD38, nicotinamide, inhibited both the CD38 enzymatic activity and differentiation. More specific blockage of CD38 expression was achieved by using morpholino antisense oligonucleotides targeting its mRNA, which produced a corresponding inhibition of differentiation as well (Munshi C B, Graeff R, Lee H C, J Biol Chem 2002 Dec. 20;277(51):49453-8).

Other studies have shown an opposite effect of CD38 signaling on progenitor cell differentiation. Short term treatment of human progenitor cells with cADPR mediated a significant increase in colony size and colony output, implying a direct correlation between CD38 signaling and ex-vivo stem cell expansion (Podesta (2000) FASEB J. 14:680-690). In a more recent study reported by the same group, the effects of cADPR on engraftment of hemopoietic stem cells into irradiated NOD/SCID mice were addressed (Podesta (2002) FASEB J. December 3 epub ahead of print). In this study a dual effect of cADPR on human hemopoietic progenitors was demonstrated in vivo, essentially, enhanced proliferation of committed progenitors responsible for improvement of short-term engraftment; and expansion of human stem cells with increased long-term human engraftment into secondary recipients. Hence, these results suggest the use of cADPR to achieve long-term expansion of human stem cells.

Thus, the prior art studies conducted on human stem cells, thus far, teach the use of cADPR, a product resulting from CD38 catalysis, for ex-vivo or in-vivo expansion of human stem cells.

Recently, Park et al demonstrated that the PI 3-kinase inhibitor LY294002 prevented monocyte differentiation and, in a dose dependent manner, induced apoptosis in PMA-induced HL-60 cells (Park et al., Immunopharmacol Immunotoxicol. 2002 May;24:211-26). Similarly, Birkenkamp et al showed that inhibition of PI 3-kinase activity in early blast AML cells resulted in inhibition of IL-1 induced cell proliferation (Birkenkamp, et al, Exp Hematol 2000;28: 1239-49). However, Kitanaka et al reported that human B-cell progenitors treated with PI 3-kinase inhibitors exhibited a reversal of CD38-ligation-induced growth inhibition (Kitanaka et al J Immunol, 1997;159:184-92), indicating that the role of PI 3-kinase signalling in CD38-associated stem cell and progenitor growth and development is as yet unclear. Indeed, Ptasznik et al (U.S. Pat. No. 6,413,773, incorporated herein by reference) have disclosed the use of inhibitors of PI 3-kinase for induction of differentiation of stem cell populations. Using undifferentiated human fetal cells, Ptasznik et al induced morphological and functional endocrine differentiation, associated with an increase in mRNA levels of insulin, glucagon, and somatostatin, as well as an increase in the insulin protein content and secretion response to secretagogues by blockading PI 3-kinase activity with LY294002. Downregulating modulation of PI 3-kinase also increased the proportion of pluripotent precursor cells coexpressing multiple hormones and the total number of terminally differentiated cells originating from these precursor cells.

In stark constrast to the prior art, while reducing the present invention to practice, the present inventors have surprisingly uncovered that inhibition of PI 3-kinase activity or expression results in ex-vivo expansion of human stem cells and, at the same time, in limited differentiation of the cells.

Evidently, the prior art described above teaches away from the present invention.

Retinoid receptors such as RAR, RXR and VDR and their agonists, such as Vitamin A and it's active metabolites and Vitamin D and it's active metabolites are involved in the regulation of gene expression pathways associated with cell proliferation and differentiation.

Vitamin D, which was shown to be a differentiation inducer of myelomonocytic cells, transduces its signals via induction of hetrodimerization of the RXR-VDR retinoid receptors (28), whereas RAR-RXR or RXR-RXR hetrodimerization is essential for retinoids inducing granulocytic differentiation.

It was shown that the retinoids are essential for the maintenance of normal differentiation in many tissues.

The disruption of retinoic acid receptor (RAR) activity characterizes the human acute promyelocytic leukemia (APL) and is associated with a block of granulocytic differentiation, indicating that RARs are critical regulators of normal myeloid differentiation.

Although the above evidence clearly portrays an important role for RARs in regulating myelopoiesis, several critical questions remain unanswered. If RAR activity is ligand concentration-dependent, then what is the mechanism by which RAR activity regulates myeloid differentiation of cells that are exposed to the uniform “physiological” concentrations of retinoids that are presumably present in blood and bone marrow? Most importantly from a clinical standpoint, why do only the acute pro-myelocytic leukemic cells (APL) exhibit a dramatic response to retinoids while the other 90% of acute myelogenous leukemias do not, even though these other acute myelogenous leukemias express normal RARs (40)?

The biological effect of retinoids and retinoid receptors on normal, non-leukemic, hematopoietic stem cells was reported by Purton et al. (41).

Purton et al. (41) demonstrated that pharmacological levels (1 μmol) of all-trans-retinoic-acid (ATRA) enhanced the generation of colony-forming cell (CFC) and colony-forming unit-spleen (CFU-S) in liquid suspension cultures of Lin⁻ c-kit⁺ Sca-1⁺ murine hematopoietic precursors. Purton et al. (41) further investigated the effects of ATRA as well as an RAR antagonist, AGN 193109, on the generation of transplantable cells, including pre-CFU-S, short-term repopulating stem cells (STRCs), and long-term repopulating stem cells (LTRCs). Purton et al. (41) demonstrated that ATRA enhanced the ex-vivo maintenance and production of competitive repopulating STRCs and LTRCs from Lin⁻c-kit⁺ Sca-1⁺ cells cultured in liquid suspension for 14 days. In addition, ATRA prevented the differentiation of these primitive stem cells into more mature pre-CFU-S during the 14 days of culture. In marked contrast, Lin⁻ c-kit⁺ Sca-1⁺ cells cultured with AGN 193109, an RAR antagonist, for 7 days had virtually no short- or long-term repopulating ability, but displayed an approximately 6-fold increase in the pre-CFU-S population. Purton et al. (41) concluded from these studies that the agonist to RAR, namely retinoic acid, enhances the maintenance and self-renewal of short- and long-term repopulating stem cells. In contrast, the RAR antagonist AGN 193109 abrogates reconstituting ability, most likely by promoting the differentiation of the primitive stem cells. Purton et al. (41) argue that these results imply an important and unexpected role of retinoids in regulating hematopoietic stem cell differentiation (41).

Whereas retinoids accelerates the growth and differentiation of granulocyte progenitors in cytokine-stimulated cultures of purified CD34⁺ cells (42), at the stem cell level, the retinoids show an opposite effect.

Although in a non-hematopoietic tissue, but in accordance with Purton et al. (41), Kamei also demonstrated that retinoids, especially all-trans-retinoic-acid, inhibit the differentiation of pre-adipose cells (43).

Hence, in the hematopoietic system, nuclear retinoid receptors were strongly implicated in pathways controlling and promoting downstream differentiation of lineage-committed cells. As was shown in detail for several leukemia cell line models, such as HL-60, NH4, and 32D, which are lineage committed cells that are blocked at the myeloblast or promyelocytic stage of differentiation, inactivation of these receptors by specific antagonists, antisense or transduction with truncated receptors is associated with inhibition of induced granulocytic and monocytic differentiation.

In contrast to normal cells, in leukemia there is a disruption between regulatory pathways controlling cell proliferation and differentiation. These pathways are strictly coupled in normal cells. The only exception in which these two processes, proliferation and commitment to differentiation are not coupled, is the self-renewal proliferation pathway of the stem cells. Therefore, all the above studies do not teach the role of retinoid receptors at the stem cell level altogether (19, 22, 64).

While reducing the present invention to practice it was demonstrated that retinoic acid antagonists, when added to ex-vivo hematopoietic or hepatocyte cultures for only a limited, short-term period, enable extended long-term expansion of self-renewable stem cells.

The antagonists did not have any significant positive or negative effect on overall cell and CD34⁺ cell expansion during the short-term cultures. In addition, CD34⁺ antigen is expressed on committed as well as multi potent stem cells. Only a small fraction of the entire CD34⁺ cell population, the CD34⁺/CD38⁻ and CD34⁺/Lin⁻ cells, belong to the stem and early progenitor cell compartment.

Analysis of the content of these two rare subpopulations in two weeks ex-vivo cultures revealed that cultures supplemented with a RAR antagonist contained higher percentages of CD34⁺/CD38⁻ cells and CD34⁺/Lin⁻ cells as compared to cultures treated only with the early acting cytokines Thrombopoietin (TPO), interleukin-6 (IL-6), an FLT-3 ligand and stem cell factor (SCF). The antagonist completely abolished the expression of the CD38 antigen. Also there was inhibition of a variety of other lineage-specific (Lin) antigens. The effect of the antagonist is specific and it is apparently targeted to key regulatory genes located at the checkpoint of self-renewal and commitment to differentiation decision. These conclusions are derived from the results described herein in the Examples section, showing that the RAR antagonist down regulates only the expression of differentiation associated antigens, but not of antigens associated with stem cell phenotype such as the CD34 antigen. The percentages and absolute numbers of CD34⁺ cells were not affected by the antagonist during the short-term culture.

Further support for antagonist-specific effects on regulatory events of self-renewal and commitment to differentiation comes from experiments conducted herein with primary and passaged hepatocyte cultures. Primary cultures incubated with the antagonists revealed an increase in the proportion of cells producing α-fetoprotein, and in the number of histologically distinct oval cells, events associated with proliferation of early hepatocyte populations.

These early hepatocyte populations persisted for at least 3 weeks in culture, even in the absence of supplemental cytokines, a most unprecedented finding. Furthermore, supplementation of the cultures with growth factors had no effect on the proliferation of early hepatocyte populations, however RAR antagonist treatment enabled expansion of this population even following first passage, and demonstrated significantly expanded hepatocyte populations following second passage, further indicating a role for antagonists in cellular self-renewal capability.

In addition to its effect on short-term cultures, while reducing the present invention to practice, it was demonstrated that short-term treatment with the antagonist molecule also enabled the long-term ex-vivo expansion and self-renewal of stem cells, e.g. CD34⁺/Lin⁻ and CD34⁺38⁻ cells. Interestingly, limited exposure to the antagonist was sufficient to produce a significant and impressive prolongation of the long and extended long-term cultures as demonstrated by FACS analysis of stem cells and the functional LTC-CFUc. During the long and extended long-term cultures, the content of CFUc and CFU-mix impressively increased as compared to the content of CFU in cultures treated only with the cytokines, which actually decreases during the long-term cultures. In fact, many of the control cultures were unable to maintain any CFU potential in the long and extended long-term culture. In contrast to cultures treated for 3 weeks with the antagonist, which showed a dramatic and continuous increase of CFU-mix during the extended long-term culture period, cytokine-only treated cultures did not enable the expansion or even the maintenance of mix-colonies during the entire culture period. Expansion of stem cells, as revealed from the phenotype characterization, is in complete agreement with the long-term self-renewal potential as measured by the functional LTC-CFUc assay. Both assays demonstrate superior and prolonged expansion of self-renewing stem cells in cultures pulsed with the antagonist molecule.

It has been shown that RAR antagonists inhibited RA induced granulocytic differentiation of committed, promyelocytic HL-60 cells (25). It was also shown, that gene transfection of a truncated RAR inhibited the response of mouse derived myeloid leukemic cell line, 32D, to G-CSF (22). These studies, however, were performed with leukemic, lineage committed cell lines and specifically show only inhibition of granulocytic differentiation induced by RA or G-CSF. Hence, no regulation at the stem cell level can be concluded from the above studies.

As opposed to Purton et al. (41), whose teachings are described above, it is demonstrated herein, using antagonist molecules to retinoid receptors and human stem cell cultures, and inhibitors of PI 3-kinase signaling pathways, that retinoid receptors are involved in the regulation of stem cell self-renewal. It is further demonstrated herein that the addition of these molecules for only a limited, short-term period to the ex-vivo cultures media enables the continuous proliferation of stem cells with no alteration of their phenotype for extended time periods. Furthermore, these effects of retinoic acid receptor antagonists did not involve any cell transformation and do not result in any cell line formation.

Opposite to cell lines occasionally obtained by transduction with a truncated, dominant negative RAR (22-23), it is shown herein that, whether the antagonist was supplemented for only the first two to three weeks or continuously for the entire culture period, all cultured cells underwent normal myeloid, erythroid and lymphoid differentiation and completely lost any cell proliferation ability 16-18 weeks after the initiation of the cultures.

As opposed to genetic modifications obtained by transduction procedures that induce infinitive alterations in gene expression and cell functions (unless the transduced gene is shut off), continuous treatment with the RAR antagonist did not result in infinitive expansion or maintenance of CD34⁺/Lin⁻ phenotype. Therefore, the mechanism of activity of a dominant negative receptor is very different than the mechanism of RAR antagonist molecules. Additional supportive data of a different mode of action comes from experiments demonstrating that cells transduced with a dominant negative RAR remain immature even in the presence of a differentiation-inducers (22), which definitely is not the case with normal, non leukemic cells treated with an RAR antagonist.

Starting with normal mouse derived bone marrow (BM) cells and following transduction with a truncated RAR receptor, Collins (23) occasionally obtained a mouse-stem cell line. However, using the same mouse-derived cells and an RAR antagonist, Purton et al. (41) demonstrated that an RAR antagonist accelerated stem cell differentiation, whereas retinoic acid supported ex-vivo expansion of stem cells (41). These data provided by Purton et al. (41) and Collins (23) favor the existence of two different, unrelated mechanisms as herein discussed.

In addition to the retinoid receptors effect on hematopoietic tissue, it was demonstrated that receptors belonging to the retinoid receptor family are involved in differentiation pathways controlling normal embryogenesis as well as adult tissues development.

Multiple fetal anomalies occur in vitamin A deficient animals as well as in retinoic acid receptor gene ‘knockout’ mice, indicating that retinoic acid (an active metabolite of vitamin A) performs some essential functions in normal development. Retinoids are also long known to influence skin morphology. When antagonists to RAR are given late in gestation, 14 days post conception (dpc), they delay differentiation and maturation of the fetal skin and hair follicles in mouse (65).

RXR-alpha ablation results in epidermal interfollicular hyperplasia with keratinocyte hyperproliferation and aberrant terminal differentiation, accompanied by an inflammatory reaction of the skin. It was further shown that RXR-alpha/VDR heterodimers play a major role in controlling hair cycling, and suggested that additional signaling pathways mediated by RXR-alpha heterodimerized with other nuclear receptors are involved in postnatal hair follicle growth (66).

Taking together the above data, it is concluded that at the stem cell level, positive and negative signals via receptors belonging to the retinoid receptor family, control the physiological balance between self-renewal and commitment to differentiation of normal hematopoietic and non-hematopoietic stem cells.

The novel method of ex-vivo down-regulation of cell differentiation, enabled large expansion of embryonic and adult, hematopoietic and non-hematopoietic stem cells and may be utilized for transplantation of hematopoietic cells, gene therapy, cell replacement therapy or any other application, which requires increasing numbers of stem cells.

The utilization of a small molecule for obtaining large stem cell expansion is a feasible, economical and safe method.

Hence, in the course of the present study it was found that a series of chemical agents that bind retinoic acid, retinoid X and/or Vitamin D receptors interfere with proper receptor signaling. This interference can reversibly inhibit (delay) the process of ex-vivo differentiation of stem cells, thereby stimulating and prolonging active ex-vivo stem cell expansion.

Downstream signal transduction imposed by the above nuclear receptors may be abrogated by inhibition of phosphatidylinositol 3-kinase (PI 3-kinase), which is an obligatory factor for proper receptor signaling.

As is described in the background section above, PI 3-kinase, which is located in the cell nuclei, is obligatory for RA and VitD induction of leukaemic cell differentiation, as was demonstrated in HL-60 and THP, myeloid leukaemic cells. Following induction of HL-60 cells to granulocytic differentiation by all-trans-retinoic acid, increase in the amount of PI 3-kinase, particularly at the nuclear level was observed. PI 3-kinase critical function in the activation of nuclear receptors such as VDR was demonstrated following treatment with 1α, 25-dihydroxyvitamin D₃ (D₃) which was associated with rapid and transient increases in PI 3-kinase activity as well as with maturation of myeloid cells and surface expressions of CD14 and CD11b, markers of cell differentiation.

As is further described in the background section above, induction of CD14 and CD11b expression in response to D₃ as well as RA induction of HL-60 cell differentiation and up regulation of CD38+ protein expression were abrogated by (a) the PI 3-kinase inhibitors LY294002 and wortmannin; (b) antisense oligonucleotides to mRNA for the p110 catalytic subunit of PI 3-kinase; (c) a dominant negative mutant of PI 3-kinase; and (d) transfection with an antisense fragment of p85α. Inhibition of PI 3-kinase activity prevented the differentiative process of leukaemic cells, indicating that PI 3-kinase activity plays an essential role in promoting granulocytic differentiation

Similarly, and as further described in the background section above, LY294002 and wortmannin, IP 3-kinase inhibitors, inhibited D₃-induced expression of both CD14 and CD11b in peripheral blood monocytes. Western blots and in vitro kinase assays carried out on immunoprecipitates of the VDR showed that D₃ treatment brought about formation of a complex containing both PI 3-kinase and the VDR.

Similarly, and as further described in the background section above several differentiation-inducing agents activate PI3-kinase and the inhibition of the PI3K/p70S6K pathway blocks the process of differentiation in these cell lines (Marcinkowska, E Postepy Hig Med Dosw 1999;53(2):305-13)

These findings reveal a novel, nongenomic mechanism of hormone action regulating monocyte differentiation, in which vitamin D₃ activates a VDR and PI 3-kinase-dependent signaling pathway.

Taking the above data, it may be postulated that RA and VitD enhanced cell differentiation via induction of dimerization of the nuclear receptors, RAR&RXR and RXR&VDR, respectively, which, following activation, recruit an additional protein, PI 3-kinase. Downstream signal transduction by the nuclear hetrodimers appears to be PI 3-kinase depended. Only in the presence of the active form of PI 3-kinase, these receptors will further control gene expression and as a result, will induce and accelerate cell differentiation. Inhibition of PI 3-kinase enzymatic activity by site specific PI 3-kinase inhibitors, down regulated CD38 expression as well as abrogated leukemic cell differentiation induced by either RA or VitD.

Compounds that specifically inhibited RA and VitD induction of late stages of differentiation of leukemic cells as well as down-regulate CD38 expression, i.e., RAR, RXR and VDR antagonists, are shown herein to also inhibit cytokine induction of normal, stem/early progenitor cell differentiation. Therefore, inhibition of PI 3-kinase activity and/or expression by site specific inhibitors is anticipated to result in inhibition of CD34+ cell differentiation, similar or better than RAR antagonists and/or nicotinamide.

As is further described in the background section above, copper ions strongly activate PI 3-kinase. As a consequence, at high cellular copper, PI 3-kinase will be very active whereas at low cell copper content PI 3-kinase will lose, at least part, its activity. Indeed, it is demonstrated herein that modulation of cellular copper by copper chelators either accelerated or reduced the rate of cell differentiation.

Copper hence modulates cell proliferation and differentiation via activation (at high intracellular copper content) or deactivation (at low intracellular copper content) of PI 3-kinase which is an obligatory factor in up regulation of CD38 gene expression and cell differentiation.

Under low copper content (imposed by supplementing the culture media with a copper chelator such as tetraethylenpentamine—TEPA) PI 3-kinase is less active, resulting in a delay in cell differentiation. On the other hand, at high cell copper content, PI 3-kinase is strongly activated, resulting in acceleration of cell differentiation.

Taken together, it is demonstrated that site-specific reagents, such as the RAR antagonists (that switched off CD38 gene expression), the nicotinamide (that abrogated its biological enzymatic activity), as well as reduction in the enzymatic activity of PI 3-kinase by reduction in cell copper content that results in less effective signals via the retinoid receptors, all strongly inhibited CD34+ cell differentiation.

Inhibitors of CD38, although active at different cellular levels, are very potent inhibitors of stem cell differentiation. Inhibition of CD38 either at the transcriptional level by RAR antagonists, PI 3-kinase specific inhibitors, de-activation of PI 3-kinase by copper chelators, as well as inhibition of CD38 enzymatic activity (ADP ribosyl cyclase) resulted in inhibition of CD34+ cell differentiation and elevation in ex vivo expansion of early progenitor cells.

It is postulated that regulation of CD38 via the PI 3-kinase signaling pathway is a critical event in stem cell determination, either in the direction of self-renewal or of differentiation. Experiments combining different reagents, active at different cellular targets, demonstrated neither additive nor synergistic effect. These results support the PI 3-kinase-mediated regulation of CD38 protein and it's biological function as a casual event in regulation of stem cells self-renewal.

Without wishing to be limited to a single hypothesis, it will be understood that PI 3-kinase activity stands at a crucial intersection of differentiation and proliferation signal transduction in the stem cell, and that the novel effect of downregulation of PI 3-kinase signaling pathways on stem cell differentiation described herein is the result of modification, by reduction of PI 3-kinase activity, of the effects of a plurality of stem cell effective factors, such as cytokines, receptor agonists, etc.

This newly discovered effect of modulation of retinoid receptors signal transduction as well as CD38 biological activity, via PI 3-kinase signaling, is applicable for maximizing the ex-vivo expansion of various types of cells including hematopoietic cells, hepatocytes and embryonic stem cells. Such ex-vivo expanded cells can be applied in several clinical situations. The following lists a few.

Hematopoietic cell transplantation: Transplantation of hematopoietic cells has become the treatment of choice for a variety of inherited or malignant diseases. While early transplantation procedures utilized the entire bone marrow (BM) population, recently, more defined populations, enriched for stem cells (CD34⁺ cells) have been used (44). In addition to the marrow, such cells could be derived from other sources such as peripheral blood (PB) and neonatal umbilical cord blood (CB) (45). Compared to BM, transplantation with PB cells shortens the period of pancytopenia and reduces the risks of infection and bleeding (46-48).

An additional advantage of using PB for transplantation is its accessibility. The limiting factor for PB transplantation is the low number of circulating pluripotent stem/progenitor cells.

To obtain enough PB-derived stem cells for transplantation, these cells are “harvested” by repeated leukophoresis following their mobilization from the marrow into the circulation by treatment with chemotherapy and cytokines (46-47). Such treatment is obviously not suitable for normal donors.

The use of ex-vivo expanded stem cells for transplantation has the following advantages (49-50):

It reduces the volume of blood required for reconstitution of an adult hematopoietic system and may obviate the need for mobilization and leukophoresis (46).

It enables storage of small number of PB or CB stem cells for potential future use.

In the case of autologous transplantation of recipients with malignancies, contaminating tumor cells in autologous infusion often contribute to the recurrence of the disease (46). Selecting and expanding CD34⁺ stem cells will reduce the load of tumor cells in the final transplant.

The cultures provide a significant depletion of T lymphocytes, which may be useful in the allogeneic transplant setting for reducing graft-versus-host disease.

Clinical studies indicate that transplantation of ex-vivo expanded cells derived from a small number of PB CD34⁺ cells can restore hematopoiesis in recipients treated with high doses of chemotherapy, although the results do not yet allow firm conclusions about long term in-vivo hematopoietic capabilities of these cultured cells (46-47).

For successful transplantation, shortening of the duration of the cytopenic phase, as well as long-term engraftment, is crucial. Inclusion of intermediate and late progenitor cells in the transplant could accelerate the production of donor-derived mature cells thereby shortening the cytopenic phase. It is important, therefore, that ex-vivo expanded cells include, in addition to stem cells, more differentiated progenitor cells in order to optimize short-term recovery and long-term restoration of hematopoiesis. Expansion of intermediate and late progenitor cells, especially those committed to the neutrophilic and megakaryocytic lineages, concomitant with expansion of stem cells, should serve this purpose (51).

Such cultures may be useful in restoring hematopoiesis in recipients with completely ablated bone marrow, as well as in providing a supportive measure for shortening recipient bone marrow recovery following conventional radio- or chemo-therapies.

Prenatal diagnosis of genetic defects in scarce cells: Prenatal diagnosis involves the collection of embryonic cells from a pregnant woman, in utero, and analysis thereof for genetic defects. A preferred, non-invasive, means of collecting embryonic cells involves separation of embryonic nucleated red blood cell precursors that have infiltrated into peripheral maternal circulation. However, since the quantities of these cells are quite scarce, a further application of the present invention would be the expansion of such cells according to methods described herein, prior to analysis. The present invention, therefore, offers a means to expand embryonic cells for applications in prenatal diagnosis.

Gene therapy: For successful long-term gene therapy, a high frequency of genetically modified stem cells with transgenes stably integrated within their genome, is an obligatory requirement. In BM tissue, while the majority of cells are cycling progenitors and precursors, stem cells constitute only a small fraction of the cell population and most of them are in a quiescent, non-cycling state. Viral-based (e.g., retroviral) vectors require active cell division for integration of the transgene into the host genome. Therefore, gene transfer into fresh BM stem cells is highly inefficient. The ability to expand a purified population of stem cells and to regulate their cell division ex-vivo would provide for an increased probability of their genetic modification (52).

Adoptive immunotherapy: Ex-vivo-expanded, defined lymphoid subpopulations have been studied and used for adoptive immunotherapy of various malignancies, immunodeficiencies, viral and genetic diseases (53-55).

The treatment enhances the required immune response or replaces deficient functions. This approach was pioneered clinically by Rosenberg et al. (56) using a large number of autologous ex-vivo expanded non-specific killer T cells, and subsequently ex-vivo expanded specific tumor infiltrating lymphocytes.

Functionally active, antigen-presenting cells could be grown from a starting population of CD34⁺ PB cells in cytokine-supported cultures, as well. These cells can present soluble protein antigens to autologous T cells in-vitro and, thus, offer new prospects for the immunotherapy of minimal residual disease after high dose chemotherapy. Ex-vivo expansion of antigen-presenting dendritic cells has been studied as well, and is an additional promising application of the currently proposed technology (57-59).

Ex-vivo Expansion of Non-hematopoietic Stem and Progenitor Cells:

Additional applications of the technology proposed herein include the possibility for ex-vivo expansion of non-hematopoietic stem and progenitor cells, including, for example, neural stem cells, oligodendrocyte progenitors, and the like.

Myelin disorders form an important group of human neurological diseases that are, as yet, incurable. Progress in animal models, particularly in transplanting cells of the oligodendrocyte lineage, has resulted in significant focal remyelination and physiological evidence of restoration of function (60). Future therapies could involve both transplantation and promotion of endogenous repair, and the two approaches could be combined with ex-vivo manipulation of donor tissue.

U.S. Pat. No. 5,486,359 illustrates that isolated human mesenchymal stem cells can differentiate into more than one tissue type (e.g. bone, cartilage, muscle, or marrow stroma) and provides a method for isolating, purifying, and expanding human mesenchymal stem cells in culture.

U.S. Pat. No. 5,736,396 provides methods for in-vitro or ex-vivo lineage-directed induction of isolated, culture-expanded human mesenchymal stem cells comprising mesenchymal stem cell contact with a bioactive factor effective in inducing stem cell differentiation into a lineage of choice. Further disclosed is a method including introducing culture-expanded lineage-induced mesenchymal stem cells into the original, autologous host, for purposes of mesenchymal tissue regeneration or repair.

U.S. Pat. No. 4,642,120 provides compositions for repairing defects in cartilage and bones. These are provided in gel form either as such, or embedded in natural or artificial bones. The gel comprises certain types of cells. Cells may be committed embryonal chondrocytes or any mesenchymal-origin cells which potentially can be converted to become functional cartilage cells, typically by the inclusion of chondrogenic inducing factors, in combination with fibrinogen, antiprotease and thrombin.

U.S. Pat. No. 5,654,186 illustrates that blood-borne mesenchymal cells proliferate in culture, and in-vivo, as demonstrated in animal models, and are capable of migrating into wound sites from the blood to form skin.

U.S. Pat. No. 5,716,411 reveals a method of skin regeneration of a wound or burn in an animal or human. This method comprises the steps of initially covering the wound with a collagen glycosaminoglycan (GC) matrix, facilitating mesenchymal cell and blood vessel infiltration from healthy underlying tissue within the grafted GC matrix. Subsequently a cultured epithelial autograft sheet grown from epidermal cells taken from the animal or human at a wound-free site is applied on the body surface. The resulting graft has excellent inclusion rates and has the appearance, growth, maturation and differentiation of normal skin.

U.S. Pat. No. 5,716,616 provides methods for treating recipients suffering from diseases, disorders or conditions characterized by bone, cartilage, or lung defects. The methods comprise intravenous administration of stromal cells isolated from normal, syngeneic individuals, or intravenous administration of stromal cells isolated from the recipient subsequent to correction of the genetic defect in the isolated cells. Methods of introducing genes into a recipient individual are also disclosed. The methods comprise obtaining a bone marrow sample from either the recipient individual or a matched syngeneic donor and isolating adherent cells from the sample. Once isolated, donor adherent cells are transfected with a gene and administered to a recipient individual intravenously. Compositions comprising isolated stromal cells that include exogenous genes operably linked to regulatory sequences are disclosed, as well.

In each of the above examples, non-hematopoietic stem and progenitor cells are used as an external source of cells for replenishing missing or damaged cells of an organ. Such use requires high levels of stem and progenitor cell expansion for successful application of the proposed therapies. Because of this pressing need for large numbers of expanded stem and progenitor cell populations, the methods and applications of the present invention address a critical niche in any of the methods disclosed in the above U.S. patents.

Additional Examples for Both ex-vivo and in-vivo Applications:

Additional applications of stem and progenitor cell expansion include skin regeneration, hepatic regeneration, muscle regeneration and stimulation of bone growth for applications in osteoporosis.

Mobilization of bone marrow stem cells into peripheral blood (peripheralization): Effects of modulators of PI 3-kinase activity or gene expression have additional in-vivo applications. As mentioned above, PB-derived stem cells for transplantation are “harvested” by repeated leukophoresis following their mobilization from the marrow into the circulation by treatment with chemotherapy and cytokines (46-47).

The use of chemotherapy is, of course, not suitable for normal donors. Administration of antagonists, into the donor could increase the marrow stem cell pool, which is then mobilized into the periphery by endogenous or injected G-CSF.

Stimulation of fetal hemoglobin production: Increased fetal hemoglobin has been shown to ameliorate clinical symptoms in recipients suffering β-hemoglobinopathies, such as sickle cell anemia and β-thalassemia (61).

Fetal hemoglobin, which normally comprises 1% of the total hemoglobin, becomes elevated in accelerated erythropoiesis (e.g., following acute hemolysis or hemorrhage or administration of erythropoietin) (62).

It has been suggested that this phenomenon is associated with acceleration of the maturation/differentiation process of erythroid precursors (63). Administration of modulators of PI 3-kinase activity or gene expression to recipients with β-hemoglobinopathies might first increase and synchronize their early erythroid progenitor pool, by blocking progenitor differentiation.

Following cessation of administration of the drug and its removal from the body, this early population then might undergo accelerated maturation, which may result in an elevated production of fetal hemoglobin.

US Patent Publication No: 03/0215445, to Serrero, describes the role of a glycoprotein GP88 secreted by malignant cells in malignant growth processes, mediated by the PI 3-kinase pathway. The authors disclose the use of anti-GP88 antibodies, anti-GP88 antisense constructs, and other GP88 antagonists, inhibiting the PI3-kinase signaling pathway, for the inhibition of malignant hematopoietic cell growth, especially B-cell malignancies such as lymphocytic leukemia and multiple myeloma. However, no mention is made of expansion of stem cells or stem cell populations using PI 3-kinase inhibitors.

In vivo administration of inhibitors of signalling pathways is well known in the art. The tyrosine kinase inhibitor imatinib mesylate (ST1571), or Gleevec (Novartis Pharma AG, New Jersey, USA), has received FDA approval for treatment of Chronic Myeloid Leukemia, and has been used in the clinical setting since 2003. Genistein and diazen, isoflavones with specific tyrosine kinase inhibiting activity, have been used for treatment of a wide variety of diseases in humans: breast and prostate cancer, postmeopausal syndrome, osteoporosis and cardiovascular disease, in addition to their antiphotocarcinogenic and antiphotoageing properties (for a review of genistein and diazen see Cos, et al Planta Med 2003; 69:589-99). Indeed, inhibition of the PI 3-kinase signaling pathway, and its angiogenic effects, has been proposed as a treatment target for a number of diseases associated with abnormal cell proliferation and tumorogenicity, as recently reviewed by Robert Mocharnuk et al:

“First, malignant GBM cells promote tumorigenesis via spontaneous EGFR signaling. Second, EGFR kinase inhibitors may be less effective in certain tumor types that overexpress EGFR, especially when the PI3/AKT signaling pathway is still activated by a PTEN mutation. Thus, simply inhibiting EGFR activity may be insufficient to inhibit the downstream putative effector molecule (PI3/AKT) when there is still an activating PTEN mutation. This would suggest the need to develop inhibitors of PI3/AKT or to develop 2 or more drugs that inhibit upstream (EGFR) and downstream (PI3/AKT) phosphorylation as a means to achieve tumor control.”(Mocharnuk et al, Novel Approaches to the Treatment of Cancer, www.medscape.com, November 2002).

The following description provides more details relating to specific aspects and embodiments of the present invention.

According to one aspect of the present invention there is provided a method of ex-vivo expanding and inhibiting differentiation of a population of stem cells. The method according to this aspect of the present invention is effected by providing the stem cells with ex-vivo culture conditions for ex-vivo cell proliferation and, at the same time, ex-vivo providing the cells with an effective amount of a modulator of PI 3-kinase activity, or of an expression of a gene encoding a PI 3-kinase, thereby ex-vivo expanding and inhibiting differentiation of the stem cells.

As used herein, the phrase “stem cells” refers to pluripotent cells that, given the right growth conditions, can develop to any cell lineage present in the organism from which they were derived. The phrase, as used herein, refers both to the earliest renewable cell population responsible for generating cell mass in a tissue or body and the very early progenitor cells, which are somewhat more differentiated, yet are not committed and can readily revert to become a part of the earliest renewable cell population. Methods of ex-vivo culturing stem cells of different tissue origins are well known in the art of cell culturing. To this effect, see for example, the text book “Culture of Animal Cells—A Manual of Basic Technique” by Freshney, Wiley-Liss, N.Y. (1994), Third Edition, the teachings of which are hereby incorporated by reference.

As used herein the term “inhibiting” refers to slowing, decreasing, delaying, preventing, reversing or abolishing. Similarly, the term “downregulation” refers to reducing, partially or totally, the indicated activity or expression. It will be appreciated, in the context of the present invention, that, due to their crucial metabolic importance, the downregulation of PI 3-kinase signalling pathways, will preferrably be partial downregulation.

As used herein the term “differentiation” refers to relatively generalized or specialized changes during development. Cell differentiation of various lineages is a well-documented process and requires no further description herein. As used herein the term differentiation is distinct from maturation which is a process, although some times associated with cell division, in which a specific cell type mature to function and then dies, e.g., via programmed cell death.

The phrase “cell expansion” is used herein to describe a process of cell proliferation substantially devoid of cell differentiation. Cells that undergo expansion hence maintain their cell renewal properties and are oftentimes referred to herein as renewable cells, e.g., renewable stem cells.

As used herein the term “ex-vivo” refers to a process in which cells are removed from a living organism and are propagated outside the organism (e.g., in a test tube). As used herein, the term “ex-vivo”, however, does not refer to a process by which cells known to propagate only in-vitro, such as various cell lines (e.g., HL-60, MEL, HeLa, etc.) are cultured. Such cells proliferate spontaneously in culture, without differentiation, in the absence of cytokines or specific differentiation-inhibiting factors, and are “committed” (differentiated), and not undifferentiated stem or progenitor cells, as taught and claimed for the present invention. Such cell lines are, by definition, “blocked” in their ability to undergo spontaneous differentiation, and as such cannot constitute a model for demonstrating effects of PI 3-kinase on hematopoietic stem cells and/or progenitor cells. In other words, cells expanded ex-vivo according to the present invention do not transform into cell lines in that they eventually undergo differentiation.

Providing the ex-vivo grown cells with conditions for ex-vivo cell proliferation include providing the cells with nutrients and preferably with one or more cytokines, as is further detailed hereinunder.

As mentioned hereinabove, concomitant with treating the cells with conditions which allow for ex-vivo the stem cells to proliferate, the cells are short-term treated or long-term treated to reduce the expression and/or activity of PI 3-kinase.

In one embodiment of the invention, reducing the activity of PI 3-kinase is effected by providing the cells with an modulator of PI 3-kinase that inhibits PI 3-kinase catalytic activity (i.e., a PI 3-kinase inhibitor).

As used herein a “modulator capable of downregulating PI 3-kinase activity or gene expression” refers to an agent which is capable of down-regulating or suppressing PI 3-kinase activity in stem cells.

An inhibitor of PI 3-kinase activity according to this aspect of the present invention can be a “direct inhibitor” which inhibits PI 3-kinase intrinsic activity or an “indirect inhibitor” which inhibits the activity or expression of PI 3-kinase signaling components (e.g., the Akt and PDK1 signaling pathways) or other signaling pathways which are effected by PI 3-kinase activity.

According to presently known embodiments of this aspect of the present invention, wortmannin and LY294002 are preferred PI 3-kinase inhibitors.

Hence, in one embodiment, the method according to this aspect of the present invention is effected by providing known PI 3-kinase inhibitors, such as wortmannin, LY294002, and active derivatives thereof, as described in, for example, U.S. Pat. Nos. 5,378,725, 5,480,906, 5,504,103, and in International Patent Publications WO 03072557, and WO 9601108, which are incorporated herein by reference, and by the specific PI 3-kinase inhibitors disclosed in US Patent Publication 20030149074 to Melese et al., also incorporated herein by reference.

Phosphatidylinositol 3-kinase inhibitors are well know to those of skill in the art. Such inhibitors include, but are not limited to Ly294002 (Calbiochem Corp., La Jolla, Calif.) and wortmannin (Sigma Chemical Co., St. Louis Mo.) which are both potent and specific PI3K inhibitors. The chemical properties of Ly294002 are described in detail in J. Biol., Chem., (1994) 269: 5241-5248. Briefly, Ly294002, the quercetin derivative, was shown to inhibit phosphatidylinositol 3-kinase inhibitor by competing for phosphatidylinositol 3-kinase binding of ATP. At concentrations at which LY294002 fully inhibits the ATP-binding site of PI3K, it has no inhibitory effect against a number of other ATP-requiring enzymes including PI4-kinase, EGF receptor tyrosine kinase, src-like kinases, MAP kinase, protein kinase A, protein kinase C, and ATPase.

LY294002 is very stable in tissue culture medium, is membrane permeable, has no significant cytotoxicity, and at concentrations at which it inhibits members of PI3K family, it has no effect on other signaling molecules.

Phosphatidylinositol 3-kinase, has been found to phosphorylate the 3-position of the inositol ring of phosphatidylinositol (PI) to form phosphatidylinositol 3-phosphate (PI-3P) (Whitman et al. (1988) Nature, 322: 664-646). In addition to PI, this enzyme also can phosphorylate phosphatidylinositol 4-phosphate and phosphatidylinositol 4,5-bisphosphate to produce phosphatidylinositol 3,4-bisphosphate and phosphatidylinositol 3,4,5-trisphosphate (PIP3), respectively (Auger et al. (1989) Cell, 57: 167-175). PI 3-kinase inhibitors are materials that reduce or eliminate either or both of these activities of PI 3-kinase. Identification, isolation and synthesis of such inhibitors is disclosed in U.S. Pat. No. 6,413,773 to Ptasznik et al.

The phrase “active derivative” refers to any structural derivative of wortmannin or LY294002 having a PI 3-kinase downregulatory activity, as measured, for example, by catalytic activity, binding studies, etc, in vivo or in vitro.

Alternatively, a modulator downregulating PI 3-kinase activity or gene expression according to this aspect of the present invention can be an activity neutralizing anti-PI 3-kinase antibody which binds, for example to the PI 3-kinase catalytic domain, or substrate binging site, thereby inhibiting PI 3-kinase catalytic activity. It will be appreciated, though, that since PI 3-kinase is an intracellular protein measures are taken to use modulators which may be delivered through the plasma membrane. In this respect a fragmented antibody such as a Fab fragment (described hereinunder), or a genetically engineered ScFv is preferably used.

The term “antibody” as used in this invention includes intact molecules as well as functional fragments thereof, such as Fab, F(ab′)₂, and Fv that are capable of binding to macrophages. These functional antibody fragments are defined as follows:

Fab, the fragment which contains a monovalent antigen-binding fragment of an antibody molecule, can be produced by digestion of whole antibody with the enzyme papain to yield an intact light chain and a portion of one heavy chain;

Fab′, the fragment of an antibody molecule that can be obtained by treating whole antibody with pepsin, followed by reduction, to yield an intact light chain and a portion of the heavy chain; two Fab′ fragments are obtained per antibody molecule;

(Fab′)₂, the fragment of the antibody that can be obtained by treating whole antibody with the enzyme pepsin without subsequent reduction; F(ab′)₂ is a dimer of two Fab′ fragments held together by two disulfide bonds;

Fv, defined as a genetically engineered fragment containing the variable region of the light chain and the variable region of the heavy chain expressed as two chains; and

Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

Antibody fragments according to the present invention can be prepared by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. Anti-PI 3-kinase antibodies are available commercially, for example, monoclonal human recombinant anti-PI 3-kinase (A.G. Scientific San Diego Calif.), anti-PI 3-kinase p85 subunit human monoclonal antibodies (Abcam, Cambridge, UK and Serotec, Inc), and anti-p85 N-SH2 domain monoclonal antibody (Upstate Biotechnology, Lake Placid, N.Y.).

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv or scFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11: 1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins recipient antibody in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Alternatively, the method according to this aspect of the present invention can be effected by providing the ex-vivo cultured stem cells with a modulator capable of downregulating a PI 3-kinase activity or an expression of a gene encoding a PI 3-kinase, the modulator selected from the group consisting of an inhibitor of PI 3-kinase catalytic activity, an antisense polynucleotide capable of specifically hybridizing with an mRNA transcript encoding PI 3-kinase, a ribozyme which specifically cleaves PI 3-kinase transcripts, coding sequences and/or promoter elements, an siRNA molecule capable of inducing degradation of PI 3-kinase transcripts, and a DNAzyme which specifically cleaves PI 3-kinase transcripts or DNA.

A modulator that downregulates PI 3-kinase expression refers to any agent which affects PI 3-kinase synthesis (decelerates) or degradation (acelerates) either at the level of the mRNA or at the level of the protein. For example, downregulation of PI 3-kinase expression can be achieved using oligonucleotide molecules designed to specifically block the transcription of PI 3-kinase mRNA, or the translation of PI 3-kinase transcripts at the ribosome, can be used according to this aspect of the present invention. In one embodiment, such oligonucleotides are antisense oligonucleotides.

Design of antisense molecules which can be used to efficiently inhibit PI 3-kinase expression must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof. Sequences suitable for use in construction and synthesis of oligonucleotides which specifically bind to PI 3-kinase mRNA, genomic DNA, promoter and/or other control sequences of PI 3-kinase are available in published PI 3-kinase nucleotide sequences, including, but not limited to, GenBank Accession Nos: AF327656 (human gamma catalytic subunit); NM006219 (human beta subunit); NM002647 (human class III); NM181524 (human p85 alpha subunit); U86453 (human p110 delta isoform); and S67334 (human p110 beta isoform).

The prior art teaches of a number of delivery strategies which can be used to efficiently deliver oligonucleotides into a wide variety of cell types (see, for example, Luft (1998) J Mol Med 76(2): 75-6; Kronenwett et al. (1998) Blood 91(3): 852-62; Rajur et al. (1997) Bioconjug Chem 8(6): 935-40; Lavigne et al. (1997) Biochem Biophys Res Commun 237(3): 566-71 and Aoki et al. (1997) Biochem Biophys Res Commun 231(3): 540-5).

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. (1999) Biotechnol Bioeng 65(1): 1-9].

Such algorithms have been successfully employed to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al. (1998) Nature Biotechnology 16, 1374-1375). Examples of antisense molecules which have been demonstrated capable of down-regulating the expression of PI 3-kinase are the PI 3-kinase specific antisense oligonucleotides described by Mood et al (Cell Signal 2004;16:631-42), incorporated herein by reference. The production of PI 3-kinase-specific antisense molecules is disclosed by Ptasznik et al (U.S. Pat. No. 6,413,773), incorporated herein by reference.

Several clinical trials have demonstrated safety, feasibility and activity of antisense oligonucleotides. For example, antisense oligonucleotides suitable for the treatment of cancer have been successfully used (Holmund et al. (1999) Curr Opin Mol Ther 1(3):372-85), while treatment of hematological malignancies via antisense oligonucleotides targeting c-myb gene, p53 and Bcl-2 had entered clinical trials and had been shown to be tolerated by patients [Gerwitz (1999) Curr Opin Mol Ther 1(3):297-306].

More recently, antisense-mediated suppression of human heparanase gene expression has been reported to inhibit pleural dissemination of human cancer cells in a mouse model [Uno et al. (2001) Cancer Res 61(21):7855-60].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation. The antisense sequences described herein can also include a ribozyme sequence fused thereto. Ribozymes suitable for use in the present invention are further described hereinbelow. Such a ribozyme sequence can be readily synthesized using solid phase oligonucleotide synthesis.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

An additional region of the antisense oligonucleotide may serve as a substrate for enzymes capable of cleaving RNA:DNA or RNA:RNA hybrids. An example for such includes RNase H, which is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense molecules of the present invention may be formed as composite structures of two or more oligonucleotides, modified oligonucleotides, as described above. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein fully incorporated by reference.

Oligonucleotides used according to this embodiment of the present invention are those having a length selected from a range of 10 to about 200 bases preferably 15-150 bases, more preferably 20-100 bases, most preferably 20-50 bases.

The oligonucleotides of the present invention may comprise heterocyclic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the intemucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374. Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. , ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

RNA interference (RNAi) is yet another approach which can be utilized by the present invention to specifically inhibit PI 3-kinase. RNA interference is a two step process. In the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA in an ATP-dependent manner. Successive cleavage events degrade the RNA to 19-21 bp duplexes (siRNA), each with 2-nucleotide 3′ overhangs [Hutvagner and Zamore (2002) Curr. Opin. Genetics and Development 12:225-232 and Bernstein (2001) Nature 409:363-366].

In the second step, termed the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore (2002) Curr. Opin. Genetics and Development 12:225-232, Hammond et al. (2001) Nat. Rev. Gen. 2:110-119, Sharp (2001) Genes. Dev. 15:485-90]. Although the mechanism of cleavage remains unresolved, research indicates that each RISC contains a single siRNA and an RNase [Hutvagner and Zamore (2002) Curr. Opin. Genetics and Development 12:225-232]. Because of the remarkable potency of RNAi, it has been suggested that the RNAi pathway employs an amplification step. Amplification could occur by copying of the input dsRNAs which would generate more siRNAs, or by replication of the siRNAs formed. Alternatively or additionally, amplification could be effected by multiple turnover events of the RISC [Hammond et al. (2001) Nat. Rev. Gen. 2:110-119, Sharp (2001) Genes. Dev. 15:485-90, Hutvagner and Zamore (2002) Curr. Opin. Genetics and Development 12:225-232]. For more information on RNAi see the following reviews Tuschl (2001) ChemBiochem. 2:239-245, Cullen (2002) Nat. Immunol. 3:597-599 and Brantl (2002) Biochem. Biophys. Act. 1575:15-25.

Synthesis of RNAi molecules suitable for use with the present invention can be effected as follows. First, the PI 3-kinase mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about a 90% decrease in cellular GAPDH mRNA and completely abolished protein level (www.ambion.com/techlib/tn/91/912.html).

Following putative target site selection, target site sequences are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those which include low G/C content, since such sequences have proven to be more effective in mediating gene silencing as compared to those having a G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Following the abovementioned methods, Czaudema et al (Nuc Acid Res 2003;31:670-82, incorporated herein by reference) successfully inhibited PI 3-kinase expression, and activity in cells, using synthetic and expressed siRNA based on the sequence of the p110 beta subunit of PI 3-kinase.

Inhibition of PI 3-kinase expression can also be effected using ribozymes. Ribozymes are being increasingly used for the sequence-specific inhibition of gene expression by the cleavage of mRNAs encoding proteins of interest [Welch et al., “Expression of ribozymes in gene transfer systems to modulate target RNA levels.” Curr Opin Biotechnol. 1998 October;9(5):486-96]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., “Ribozyme gene therapy for hepatitis C virus infection.” Clin Diagn Virol. 1998 Jul. 15;10(2-3):163-71.]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

DNAzymes can also be utilized by the present invention. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M Curr Opin Mol Ther 2002;4:119-21).

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

It will be appreciated that protein agents (e.g., antibodies) and oligonucleotide agents (ribozymes, DNAzymes, RNAi, etc) of the present invention can be expressed from a polynucleotide encoding same and provided to ex-vivo cultured stem cells employing an appropriate gene delivery vehicle/method and a nucleic acid construct as is further described hereinunder.

Expression of such constructs can be transient or stable expression. Thus, accoding to one embodiment of the present invention, providing the modulator of PI 3-kinase activity or gene expression is effected by transiently expressing the antisense polynucleotide, the ribozyme, the siRNA molecule or the DNAzyme within a stem cell. In another, preferred embodiment, the expression is stable, and providing is effected by (a) providing an expressible polynucleotide capable of expressing the antisense polynucleotide, the ribozyme, the siRNA molecule or the DNAzyme and, (b) stably integrating said expressible polynucleotide into a genome of a cell, thereby providing a modulator capable of downregulating a PI 3-kinase activity or PI 3-kinase gene expression. Suitable constructs and methods for their stable and transient expression in cells are described hereinbelow.

Examples of suitable constructs include, but are not limited to pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

As the method of ex-vivo expanding and inhibiting differentiation of a population of stem cells, according to this aspect of the present invention, is effected by modulating PI 3-kinase expression and/or activity, either at the protein level, using a PI 3-kinase inhibitor such as wortmannin, LY294002, or derivatives thereof, or at the at the expression level via genetic engineering techniques, as is detailed hereinabove, there are further provided, according to the present invention, several preferred methods of ex-vivo expanding and inhibiting differentiation of a population of stem cells.

Inhibition of PI 3-kinase activity can be effected by known PI 3-kinase inhibitors, such as wortmannin, LY294002, and derivatives thereof, as described in, for example, U.S. Pat. Nos. 5,378,725, 5,480,906, 5,504,103, and in International Patent Publications WO 03072557, and WO 9601108, which are incorporated herein by reference, and by the specific PI 3-kinase inhibitors disclosed in US Patent Publication 20030149074 to Melese et al., also incorporated herein by reference.

Final concentrations of the modulators may be, depending on the specific application, in the micromolar or millimolar ranges. For example, within about 0.1 μM to about 100 mM, preferably within about 4 μM to about 50 mM, more preferably within about 5 μM to about 40 mM. While reducing the present invention to practice, effective inhibition of CD34⁺ hematopoietic stem cells differentiation, and renewal of the CD34⁺ population was demonstrated in cells provided with PI 3-kinase inhibitor LY294002 in the range of 0.1 μM/L to 100 μM/L. Thus, in one preferred embodiment, the effective concentration of the modulator of PI 3-kinase activity is about 0.1 μM/L to 100 μM/L, more preferably 1-50 μM/L, most preferably 10-20 μM/L.

The ex-vivo expansion of populations of stem cells, according to the features described hereinabove, can be utilized for expanding a population of hematopoietic renewable stem cells ex-vivo.

Hence, according to another aspect of the present invention, there is provided a method of ex-vivo expanding a population of hematopoietic renewable stem cells ex-vivo. The method is effected by obtaining adult or neonatal umbilical cord whole white blood cells (also known in the art as mononuclear cell fraction) or whole bone marrow cells sample and providing the cells in the sample with ex-vivo culture conditions for stem cells ex-vivo cell proliferation and, at the same time, for reducing the expression and/or activity of PI 3-kinase, as is described hereinabove, thereby expanding a population of a renewable stem cells in the sample.

In still another particular embodiment of this aspect of the present invention, the method is effected by obtaining adult or neonatal umbilical cord whole white blood cells or whole bone marrow cells sample and providing the cells in the sample with ex-vivo culture conditions for stem cells ex-vivo cell proliferation and, at the same time, for reducing a capacity of the stem cells in responding to signaling pathways involving PI 3-kinase, thereby expanding a population of a renewable stem cells in the sample.

In still another particular embodiment of this aspect of the present invention, the method is effected by obtaining adult or neonatal umbilical cord whole white blood cells or whole bone marrow cells sample and providing the cells in the sample with ex-vivo culture conditions for stem cells ex-vivo cell proliferation and with a PI 3-kinase inhibitor, thereby expanding a population of a renewable stem cells in the sample.

Expanding the population of stem cells can be further utilized, according to the present invention, in in vivo settings, such that according to still another aspect of the present invention there is provided a method of in-vivo expanding a population of stem cells, while at the same time, substantially inhibiting differentiation of the stem cells in-vivo. The method, according to this aspect of the present invention is effected by administering to a subject in need thereof a therapeutically effective amount of a modulator of PI 3-kinase activity or expression of a gene encoding PI 3-kinase, the modulator selected capable of downregulating a PI 3-kinase activity or an expression of a gene encoding PI 3-kinase, according to the features described hereinabove.

In another particular embodiment of this aspect of the present invention, the method is effected by administering to a subject in need thereof a therapeutically effective amount of a modulator of PI 3-kinase activity or expression of a gene encoding PI 3-kinase, the modulator selected capable of downregulating a PI 3-kinase activity or an expression of a gene encoding PI 3-kinase, which serves for reducing a capacity of the stem cells in responding to signaling pathways involving PI 3-kinase, as is defined hereinabove.

In still another particular embodiment of this aspect of the present invention, the method is effected by administering to a subject in need thereof a therapeutically effective amount of a PI 3-kinase inhibitor.

As used herein throughout, the phrase “therapeutically effective amount” or “effective amount” refers to that amount of the agent being administered which will induce expansion of stem cells yet inhibit the differentiation thereof.

The methods described hereinabove for ex-vivo expanding stem cells populations can result, inter alia, in an expanded population of stem cells.

Thus, further according to an aspect of the present invention there is provided an ex-vivo expanded population of hematopoietic stem cells which comprises a plurality of cells characterized by 3-20% of the cells being reselectable CD34⁺ cells, of which at least 40% of cells are CD34⁺_{dim}, i.e., fall below the median intensity in a FACS analysis, wherein, in the reselectable CD34⁺ cells, a majority of cells which are Lin⁻ are also CD34⁺_{dim} cells. In one embodiment, the hematopoietic stem cells are derived from a source selected from the group consisting of bone marrow, peripheral blood and neonatal umbilical cord blood. In another embodiment, the population of cells has a single genetic background. In yet another embodiment, the ex-vivo expanded population of hematopoietic stem cells comprises at least N cells derived from a single donor, wherein N equals the average number of CD34⁺ cells derived from one sample of neonatal umbilical cord blood, bone marrow, or peripheral blood multiplied by 1,000. Cell surface expression of the CD34 and/or Lin markers can be determined, for example, via FACS analysis or immunohistological staining techniques. A self renewal potential of the stem cells can be determined in-vitro by long term colony formation (LTC-CFUc), as is further exemplified in the Examples section that follows, or by in-vivo engraftment in the SCID-Hu mouse model. The SCID-Hu mouse model employs C.B-17 scid/scid (SCID) mice transplanted with human fetal thymus and liver tissue or fetal BM tissue and provides an appropriate model for the evaluation of putative human hematopoietic stem cells. Because of the reconstitution of the SCID mice with human fetal tissue, the model affords the proliferation of stem cells, in this case human hematopoietic stem cells to proliferate, and function in the hematopoietic microenvironment of human origin. Mice are typically irradiated, then delivered stem cells into the grafts, and reconstitution is measured by any number of methods, including FACS and immunohistochemistry of repopulated organs (Humeau L., et al. Blood (1997) 90:3496).

Additionally, the methods described hereinabove can be utilized to produce transplantable hematopoietic cell preparations, such that according to yet another aspect of the present invention there is provided a therapeutic ex vivo cultured stem cell population, which comprises an undifferentiated hematopoietic cells expanded ex-vivo in the presence of an effective amount of an a modulator of PI 3-kinase activity or expression of a gene encoding PI 3-kinase, the modulator selected capable of downregulating a PI 3-kinase activity or an expression of a gene encoding PI 3-kinase, thereby inhibiting differentiation, as described hereinabove. It will be appreciated, in the context of the present invention, that the therapeutic stem cell population can be provided along with the culture medium containing the modulator capable of downregulating a PI 3-kinase activity or an expression of a gene encoding PI 3-kinase, isolated from the culture medium, and combined with a pharmaceutically acceptable carrier. Hence, cell populations of the invention can be administered in a pharmaceutically acceptable carrier or diluent, such as sterile saline and aqueous buffer solutions. The use of such carriers and diluents is well known in the art.

In one particular embodiment of this aspect of the present invention, the therapeutic ex vivo cultured stem cell population comprises an expanded population of hematopoietic stem cells propagated ex-vivo in the presence of an effective amount of an agent, which reduces a capacity of the stem cells in responding to PI 3-kinase signaling, substantially inhibiting differentiation of the stem cells; and a pharmaceutically acceptable carrier.

In still another particular embodiment of this aspect of the present invention, the transplantable hematopoietic cell preparation comprises an expanded population of hematopoietic stem cells propagated ex-vivo in the presence of an effective amount of a PI 3-kinase inhibitor, and a pharmaceutically acceptable carrier.

The ability of the agents of the present invention to inhibit differentiation of stem cells can be further used in various technical applications:

According to a further aspect of the present invention there is provided a method of preserving stem cells. In one embodiment, the method is effected by handling the stem cell in at least one of the following steps: harvest, isolation and/or storage, in a presence of an effective amount of a modulator of PI 3-kinase activity or expression of a gene encoding PI 3-kinase, the modulator selected capable of downregulating a PI 3-kinase activity or an expression of a gene encoding PI 3-kinase. In one embodiment, the method is effected by handling the stem cell in at least one of the following steps: harvest, isolation and/or storage, in a presence of an effective amount of a PI 3-kinase inhibitor, such as wortmannin or LY294002, a modulator of PI 3-kinase activity or expression of a gene encoding PI 3-kinase, the modulator selected capable of downregulating a PI 3-kinase activity or an expression of a gene encoding PI 3-kinase, or an anti-PI 3-kinase antibody.

According to still a further aspect of the present invention there is provided a cells collection/culturing bag. The cells collection/culturing bag of the present invention is supplemented with an effective amount of a modulator of PI 3-kinase activity or expression of a gene encoding PI 3-kinase, the modulator selected capable of downregulating a PI 3-kinase activity or an expression of a gene encoding PI 3-kinase. In one embodiment, the modulator is a PI 3-kinase inhibitor, such as wortmannin or LY294002, or an anti-PI 3-kinase antibody.

According to the present invention there is also provided a cells separation and/or washing buffer. The separation and/or washing buffer is supplemented with an effective amount of a modulator of PI 3-kinase activity or expression of a gene encoding PI 3-kinase, the modulator selected capable of downregulating a PI 3-kinase activity or an expression of a gene encoding PI 3-kinase. In one embodiment, the modulator is a PI 3-kinase inhibitor, such as wortmannin or LY294002, or an anti-PI 3-kinase antibody.

As is further detailed below, stem cells may serve to exert cellular gene therapy.

Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved: (i) ex-vivo or cellular gene therapy; and (ii) in vivo gene therapy. In ex-vivo gene therapy cells are removed from a patient, and while being cultured are treated in-vitro. Generally, a functional replacement gene is introduced into the cells via an appropriate gene delivery vehicle/method (transfection, transduction, homologous recombination, etc.) and an expression system as needed and then the modified cells are expanded in culture and returned to the host/patient. These genetically re-implanted cells have been shown to express the transfected genetic material in situ.

Hence, further according to an aspect of the present invention, there is provided a method of transducing expanded, undifferentiated stem cells with an exogene. The method, according to this aspect of the present invention, is effected by: (a) obtaining a population of stem cells to be transduced; (b) expanding and inhibiting differentiation of the stem cells by: (i) providing the stem cells with conditions for cell proliferation and (ii) providing the stem cells with an effective concentration of a modulator of PI 3-kinase activity, said modulator selected capable of downregulating a PI 3-kinase activity or an expression of a gene encoding a PI 3-kinase, thereby expanding and inhibiting differentiation of the stem cells; and (c) transducing the expanded, undifferentiated stem cells with the exogene. It will be appreciated that steps (i) and (ii) can be effected in vitro or ex vivo, and that the order of steps (b) and (c) can be reversed.

In another particular embodiment of this aspect of the present invention, step (ii) is effected by reducing a capacity of the stem cells in responding to signaling pathways involving PI 3-kinase, thereby expanding and inhibiting differentiation of the stem cells.

In a preferred embodiment, genetically modifying the cells is effected by a vector, which comprises the exogene or transgene, which vector is, for example, a viral vector or a nucleic acid vector. Many viral vectors suitable for use in cellular gene therapy are known, examples are provided hereinbelow. Similarly, a range of nucleic acid vectors can be used to genetically transform the expanded cells of the invention, as is further described below.

Accordingly, the expanded cells of the present invention can be modified to express a gene product. As used herein, the phrase “gene product” refers to proteins, peptides and functional RNA molecules. Generally, the gene product encoded by the nucleic acid molecule is the desired gene product to be supplied to a subject. Examples of such gene products include proteins, peptides, glycoproteins and lipoproteins normally produced by an organ of the recipient subject. For example, gene products which may be supplied by way of gene replacement to defective organs in the pancreas include insulin, amylase, protease, lipase, trypsinogen, chymotrypsinogen, carboxypeptidase, ribonuclease, deoxyribonuclease, triaclyglycerol lipase, phospholipase A₂, elastase, and amylase; gene products normally produced by the liver include blood clotting factors such as blood clotting Factor VIII and Factor IX UDP glucuronyl transferae, ornithine transcarbanoylase, and cytochrome p450 enzymes, and adenosine deaminase, for the processing of serum adenosine or the endocytosis of low density lipoproteins; gene products produced by the thymus include serum thymic factor, thymic humoral factor, thymopoietin, and thymosin α₁; gene products produced by the digestive tract cells include gastrin, secretin, cholecystokinin, somatostatin, serotinin, and substance P.

Alternatively, the encoded gene product is one, which induces the expression of the desired gene product by the cell (e.g., the introduced genetic material encodes a transcription factor, which induces the transcription of the gene product to be supplied to the subject).

In still another embodiment, the recombinant gene can provide a heterologous protein, e.g., not native to the cell in which it is expressed. For instance, various human MHC components can be provided to non-human cells to support engraftment in a human recipient. Alternatively, the transgene is one, which inhibits the expression or action of a donor MHC gene product normally expressed in the micro-organ explant.

A nucleic acid molecule introduced into a cell is in a form suitable for expression in the cell of the gene product encoded by the nucleic acid. Accordingly, the nucleic acid molecule includes coding and regulatory sequences required for transcription of a gene (or portion thereof) and, when the gene product is a protein or peptide, translation of the gene acid molecule include promoters, enhancers and polyadenylation signals, as well as sequences necessary for transport of an encoded protein or peptide, for example N-terminal signal sequences for transport of proteins or peptides to the surface of the cell or secretion.

Nucleotide sequences which regulate expression of a gene product (e.g., promoter and enhancer sequences) are selected based upon the type of cell in which the gene product is to be expressed and the desired level of expression of the gene product. For example, a promoter known to confer cell-type specific expression of a gene linked to the promoter can be used. A promoter specific for myoblast gene expression can be linked to a gene of interest to confer muscle-specific expression of that gene product. Muscle-specific regulatory elements, which are known in the art, include upstream regions from the dystrophin gene (Klamut et al., (1989) Mol. Cell Biol. 9: 2396), the creatine kinase gene (Buskin and Hauschka, (1989) Mol. Cell Biol. 9: 2627) and the troponin gene (Mar and Ordahl, (1988) Proc. Natl. Acad. Sci. USA. 85: 6404). Regulatory elements specific for other cell types are known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters).

Alternatively, a regulatory element, which can direct constitutive expression of a gene in a variety of different cell types, such as a viral regulatory element, can be used. Examples of viral promoters commonly used to drive gene expression include those derived from polyoma virus, Adenovirus 2, cytomegalovirus and Simian Virus 40, and retroviral LTRs.

Alternatively, a regulatory element, which provides inducible expression of a gene linked thereto, can be used. The use of an inducible regulatory element (e.g., an inducible promoter) allows for modulation of the production of the gene product in the cell. Examples of potentially useful inducible regulatory systems for use in eukaryotic cells include hormone-regulated elements (e.g., see Mader, S. and White, J. H. (1993) Proc. Natl. Acad. Sci. USA 90: 5603-5607), synthetic ligand-regulated elements (see, e.g., Spencer, D. M. et al. 1993) Science 262: 1019-1024) and ionizing radiation-regulated elements (e.g., see Manome, Y. Et al. (1993) Biochemistry 32: 10607-10613; Datta, R. et al. (1992) Proc. Natl. Acad. Sci. USA 89: 1014-10153). Additional tissue-specific or inducible regulatory systems, which may be developed, can also be used in accordance with the invention.

There are a number of techniques known in the art for introducing genetic material into a cell that can be applied to modify a cell of the invention.

In one embodiment, the nucleic acid is in the form of a naked nucleic acid molecule. In this situation, the nucleic acid molecule introduced into a cell to be modified consists only of the nucleic acid encoding the gene product and the necessary regulatory elements.

Alternatively, the nucleic acid encoding the gene product (including the necessary regulatory elements) is contained within a plasmid vector. Examples of plasmid expression vectors include CDM8 (Seed, B. (1987) Nature 329: 840) and pMT2PC (Kaufman, et al. (1987) EMBO J. 6: 187-195).

In another embodiment, the nucleic acid molecule to be introduced into a cell is contained within a viral vector. In this situation, the nucleic acid encoding the gene product is inserted into the viral genome (or partial viral genome). The regulatory elements directing the expression of the gene product can be included with the nucleic acid inserted into the viral genome (i.e., linked to the gene inserted into the viral genome) or can be provided by the viral genome itself.

Naked nucleic acids can be introduced into cells using calcium-phosphate mediated transfection, DEAE-dextran mediated transfection, electroporation, liposome-mediated transfection, direct injection, and receptor-mediated uptake.

Naked nucleic acid, e.g., DNA, can be introduced into cells by forming a precipitate containing the nucleic acid and calcium phosphate. For example, a HEPES-buffered saline solution can be mixed with a solution containing calcium chloride and nucleic acid to form a precipitate and the precipitate is then incubated with cells. A glycerol or dimethyl sulfoxide shock step can be added to increase the amount of nucleic acid taken up by certain cells. CaPO₄-mediated transfection can be used to stably (or transiently) transfect cells and is only applicable to in vitro modification of cells. Protocols for CaPO₄-mediated transfection can be found in Current Protocols in Molecular Biology, Ausubel, F. M. et al. (eds.) Greene Publishing Associates, (1989), Section 9.1 and in Molecular Cloning: A Laboratory Manual, 2nd Edition, Sambrook et al. Cold Spring Harbor Laboratory Press, (1989), Sections 16.32-16.40 or other standard laboratory manuals.

Naked nucleic acid can be introduced into cells by forming a mixture of the nucleic acid and DEAE-dextran and incubating the mixture with the cells. A dimethylsulfoxide or chloroquine shock step can be added to increase the amount of nucleic acid uptake. DEAE-dextran transfection is only applicable to in vitro modification of cells and can be used to introduce DNA transiently into cells but is not preferred for creating stably transfected cells. Thus, this method can be used for short-term production of a gene product but is not a method of choice for long-term production of a gene product. Protocols for DEAE-dextran-mediated transfection can be found in Current Protocols in Molecular Biology, Ausubel, F. M. et al. (eds.) Greene Publishing Associates (1989), Section 9.2 and in Molecular Cloning: A Laboratory Manual, 2nd Edition, Sambrook et al. Cold Spring Harbor Laboratory Press, (1989), Sections 16.41-16.46 or other standard laboratory manuals.

Naked nucleic acid can also be introduced into cells by incubating the cells and the nucleic acid together in an appropriate buffer and subjecting the cells to a high-voltage electric pulse. The efficiency with which nucleic acid is introduced into cells by electroporation is influenced by the strength of the applied field, the length of the electric pulse, the temperature, the conformation and concentration of the DNA and the ionic composition of the media. Electroporation can be used to stably (or transiently) transfect a wide variety of cell types and is only applicable to in vitro modification of cells. Protocols for electroporating cells can be found in Current Protocols in Molecular Biology, Ausubel F. M. et al. (eds.) Greene Publishing Associates, (1989), Section 9.3 and in Molecular Cloning: A Laboratory Manual, 2nd Edition, Sambrook et al. Cold Spring Harbor Laboratory Press, (1989), Sections 16.54-16.55 or other standard laboratory manuals.

Another method by which naked nucleic acid can be introduced into cells includes liposome-mediated transfection (lipofection). The nucleic acid is mixed with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J Med. Sci. 298:278; and Gould-Fogerite et al. (1989) Gene 84:429-438.

Naked nucleic acid can also be introduced into cells by directly injecting the nucleic acid into the cells. For an in vitro culture of cells, DNA can be introduced by microinjection. Since each cell is microinjected individually, this approach is very labor intensive when modifying large numbers of cells. However, a situation wherein microinjection is a method of choice is in the production of transgenic animals (discussed in greater detail below). In this situation, the DNA is stably introduced into a fertilized oocyte, which is then allowed to develop into an animal. The resultant animal contains cells carrying the DNA introduced into the oocyte. Direct injection has also been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo can be used. Such an apparatus is commercially available (e.g., from BioRad).

Naked nucleic acid can be complexed to a cation, such as polylysine, which is coupled to a ligand for a cell-surface receptor to be taken up by receptor-mediated endocytosis (see for example Wu, G. and Wu, C. H. (1988) J. Biol. Chem. 263: 14621; Wilson et al. (1992) J. Biol. Chem. 267: 963-967; and U.S. Pat. No. 5,166,320). Binding of the nucleic acid-ligand complex to the receptor facilitates uptake of the DNA by receptor-mediated endocytosis. Receptors to which a DNA-ligand complex has targeted include the transferrin receptor and the asialoglycoprotein receptor. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88: 8850; Cristiano et al. (1993) Proc. Natl. Acad. Sci. USA 90: 2122-2126). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Generally, when naked DNA is introduced into cells in culture (e.g., by one of the transfection techniques described above) only a small fraction of cells (about 1 out of 10⁵) typically integrate the transfected DNA into their genomes (i.e., the DNA is maintained in the cell episomally). Thus, in order to identify cells, which have taken up exogenous DNA, it is advantageous to transfect nucleic acid encoding a selectable marker into the cell along with the nucleic acid(s) of interest. Preferred selectable markers include those, which confer resistance to drugs such as G418, hygromycin and methotrexate. Selectable markers may be introduced on the same plasmid as the gene(s) of interest or may be introduced on a separate plasmid.

A preferred approach for introducing nucleic acid encoding a gene product into a cell is by use of a viral vector containing nucleic acid, e.g., a cDNA, encoding the gene product. Infection of cells with a viral vector has the advantage that a large proportion of cells receive the nucleic acid which can obviate the need for selection of cells which have received the nucleic acid. Additionally, molecules encoded within the viral vector, e.g., a cDNA contained in the viral vector, are expressed efficiently in cells which have taken up viral vector nucleic acid and viral vector systems can be used either in vitro or in vivo.

Defective retroviruses are well characterized for use in gene transfer for gene therapy purposes (for review see Miller, A. D. (1990) Blood 76: 271). A recombinant retrovirus can be constructed having a nucleic acid encoding a gene product of interest inserted into the retroviral genome. Additionally, portions of the retroviral genome can be removed to render the retrovirus replication defective. The replication defective retrovirus is then packaged into virions, which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM, which are well known to those skilled in the art. Examples of suitable packaging virus lines include ψCrip, ψCrip, ψ2 and ψAm. Retroviruses have been used to introduce a variety of genes into many different cell types, including epithelial cells endothelial cells, lymphocytes, myoblasts, hepatocytes, bone marrow cells, in vitro and/or in vivo (see for example Eglitis, et al. (1985) Science 230: 1395-1398; Danosand Mulligan (1988) Proc. Natl. Acad. Sci. USA 85: 6460-6464; Wilson et al. (1988) Proc. Natl. Acad. Sci USA 85:3014-3018; Armentano et al., (1990) Proc. Natl. Acad. Sci. USA 87: 6141-6145; Huber et al. (1991) Proc. Natl. Acad. Sci. USA 88: 8039-8043; Feri et al. (1991) Proc. Natl. Acad. Sci. USA 88:8377-8381; Chowdhury et al. (1991) Science 254: 1802-1805; van Beusechem et al. (1992) Proc. Natl. Acad. Sci USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89: 6482-6486), hepatocytes (Herz and Gerard (1993) Proc. Natl. Acad. Sci. USA 90: 2812-2816) and muscle cells (Quantin et al. (1992) Proc. Natl. Acad. Sci. USA 89: 2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol 57: 267). Most replication-defective adenoviral vectors currently in use are deleted for all or parts of the viral E1 and E3 genes but retain as much as 80% of the adenoviral genetic material.

Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics In Micro. And Immunol. (1992) 158: 97-129). It is also one of the few viruses that may integrate its DNA into non-dividing cells, and exhibits a high frequency of stable integration (see for example Flotte et al. (1992) Am. J. Respir. Cell. Mol. Biol. 7: 349-356; Samulski et al. (1989) J. Virol. 63:3822-3828; and McLaughlin et al. (1989) J. Virol. 62: 1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5: 3251-3260 can be used to introduce DNA into cells. A variety of nucleic acids have been introduced into different cell types using AAV vectors (see for example Hermonat et al. (1984) Proc. Natl. Acad. Sci. USA 81: 6466-6470; Tratschin et al. (1985) Mol. Cell Biol. 4: 2072-2081; Wondisford et al. (1988) Mol. Endocrinol. 2:32-39; Tratschin et al. (1984) J. Virol. 51: 611-619; and Flotte et al. (1993) J. Biol. Chem. 268: 3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone.

When the method used to introduce nucleic acid into a population of cells results in modification of a large proportion of the cells and efficient expression of the gene product by the cells (e.g., as is often the case when using a viral expression vector), the modified population of cells may be used without further isolation or subcloning of individual cells within the population. That is, there may be sufficient production of the gene product by the population of cells such that no further cell isolation is needed. Alternatively, it may be desirable to grow a homogenous population of identically modified cells from a single modified cell to isolate cells, which efficiently express the gene product. Such a population of uniform cells can be prepared by isolating a single modified cell by limiting dilution cloning followed by expanding the single cell in culture into a clonal population of cells by standard techniques.

As is discussed in detail hereinabove, ex-vivo expansion of stem cells can be advantageously utilized in hematopoietic cells transplantation or implantation. Hence, according to another aspect of the present invention there is provided a method of hematopoietic cells transplantation or implantation into a recipient. The method according to this aspect of the present invention is effected by (a) obtaining a population of hematopoietic stem cells to be transplanted; (b) ex-vivo expanding and inhibiting differentiation of the hematopoietic stem cells by: (i) ex vivo providing said stem cells with conditions for cell proliferation, and (ii) providing said stem cells with an effective concentration of a modulator of PI 3-kinase activity, said modulator selected capable of downregulating a PI 3-kinase activity or an expression of a gene encoding a PI 3-kinase; thereby expanding and inhibiting differentiation of said stem cells; and (c) transplanting or implanting the hematopoietic stem cells into a recipient.

According to a preferred embodiment of the present invention, the method according to this aspect of the present invention can be effected by providing the ex-vivo cultured stem cells with a modulator capable of downregulating a PI 3-kinase activity or an expression of a gene encoding a PI 3-kinase, the modulator selected from the group consisting of an inhibitor of PI 3-kinase catalytic activity, an antisense polynucleotide capable of specifically hybridizing with an mRNA transcript encoding PI 3-kinase, a ribozyme which specifically cleaves PI 3-kinase transcripts, coding sequences and/or promoter elements, an siRNA molecule capable of inducing degradation of PI 3-kinase transcripts, and a DNAzyme which specifically cleaves PI 3-kinase transcripts or DNA.

In another particular embodiment of this aspect of the present invention, the method is effected by (a) obtaining hematopoietic stem cells to be transplanted from a donor; (b) ex-vivo expanding and inhibiting differentiation of the hematopoietic stem cells by: (i) ex vivo providing said stem cells with conditions for cell proliferation, and (ii) providing said stem cells with an effective concentration of a modulator of PI 3-kinase activity, said modulator selected capable of downregulating a PI 3-kinase activity or an expression of a gene encoding a PI 3-kinase; thereby expanding and inhibiting differentiation of said stem cells; and (c) transplanting or implanting the hematopoietic stem cells into a recipient. In yet another embodiment, step (b) is effected by providing the stem cells with ex-vivo culture conditions for reducing a capacity of the stem cells in responding to signaling pathways involving PI 3-kinase.

The donor and the recipient can be a single individual or different individuals, for example, allogeneic individuals. When allogeneic transplantation is practiced, regimes for reducing implant rejection and/or graft vs. host disease, as well know in the art, should be undertaken. Such regimes are currently practiced in human therapy. Most advanced regimes are disclosed in publications by Slavin S. et al., e.g., J Clin Immunol (2002) 22: 64, and J Hematother Stem Cell Res (2002) 11: 265), Gur H. et al. (Blood (2002) 99: 4174), and Martelli M F et al, (Semin Hematol (2002) 39: 48), which are incorporated herein by reference.

According to yet another aspect of the present invention there is provided a method of adoptive immunotherapy. The method according to this aspect of the present invention is effected by (a) obtaining progenitor hematopoietic stem cells from a patient; (b) ex-vivo expanding and inhibiting differentiation of the hematopoietic stem cells by: (i) providing the stem cells ex vivo with conditions for cell proliferation, and (ii) providing the progenitor hematopoietic cells with an effective concentration of a modulator of PI 3-kinase activity, said modulator selected capable of downregulating a PI 3-kinase activity or an expression of a gene encoding a PI 3-kinase; thereby expanding and inhibiting differentiation of said stem cells; and (c) transplanting or implanting the progenitor hematopoietic stem cells into a recipient.

In another particular embodiment of this aspect of the present invention, step (b) of the method is effected by providing the cells with conditions for reducing a capacity of the stem cells in responding to signaling pathways involving PI 3-kinase, thereby expanding a population of the stem cells, while at the same time, substantially inhibiting differentiation of the stem cells.

According to a preferred embodiment of the present invention, the modulator selected capable of downregulating a PI 3-kinase activity or an expression of a gene encoding a PI 3-kinase is selected from the group consisting of an inhibitor of PI 3-kinase catalytic activity, an antisense polynucleotide capable of specifically hybridizing with an mRNA transcript encoding PI 3-kinase, a ribozyme which specifically cleaves PI 3-kinase transcripts, coding sequences and/or promoter elements, an siRNA molecule capable of inducing degradation of PI 3-kinase transcripts, and a DNAzyme which specifically cleaves PI 3-kinase transcripts or DNA.

The effect of the agents that reduce PI 3-kinase expression or activity used in context of the present invention is not limited to ex-vivo settings. Hence, based on the findings herein described, novel in-vivo applications for these agents are envisaged.

Hence, according to yet another aspect of the present invention there is provided a method of mobilization of bone marrow stem cells into the peripheral blood of a donor for harvesting the cells. The method according to this aspect of the present invention is effected by (a) administering to the donor an effective amount of a modulator of PI 3-kinase activity or expression of a gene encoding PI 3-kinase, the modulator selected capable of downregulating a PI 3-kinase activity or an expression of a gene encoding PI 3-kinase, and harvesting the cells by leukophoresis.

In still another particular embodiment of this aspect of the present invention, step (a) of the method is effected by administering to the donor an effective amount of an agent for reducing a capacity of the stem cells in responding to signaling pathways involving PI 3-kinase, thereby expanding a and inhibiting differentiation of a population of bone marrow cells.

Preferably, the methods of mobilization of stem cells further comprise administering to the donor at least one cytokine, preferably at least one early cytokine, which are presently used to induce cell mobilization into peripheral blood.

Further according to an aspect of the present invention there is provided a method of inhibiting maturation/differentiation of erythroid precursor cells for the treatment of a β-hemoglobinopathic patient. The method according to this aspect of the present invention is effected by administering to the patient an a modulator of PI 3-kinase activity or expression of a gene encoding PI 3-kinase, the modulator selected capable of downregulating a PI 3-kinase activity or an expression of a gene encoding PI 3-kinase, thereby expanding and inhibiting differentiation of a population of stem cells of the patient, such that upon natural removal of the modulator of PI 3-kinase from the patient, the stem cells undergo accelerated maturation, resulting in elevated fetal hemoglobin production.

The modulator used according to this method of the present invention can be an agent for abrogating or reducing a capacity of the cells in responding to PI 3-kinase signaling, a inhibitor, such as wortmannin or LY294002, or an inhibitory PI 3-kinase antibody. In another embodiment, the method is effected by further administering a cytokine to the patient.

In in-vivo settings, administration of the modulators that reduce PI 3-kinase expression or activity, e.g., PI 3-kinase inhibitors wortamnnin and LY294002, or anti-PI 3-kinase antibodies, may be by a pharmaceutical composition including same, which may further include thickeners, carriers, buffers, diluents, surface active agents, preservatives, and the like, all as well known in the art.

The pharmaceutical composition may be administered in various ways, depending on the preference for local or systemic treatment, and on the area to be treated. Administration may be done topically (including opthalmically, vaginally, rectally, intranasally), orally, by inhalation, or parenterally, for example by intravenous drip or intraperitoneal, subcutaneous, subdural, intramuscular or intravenous injection, or via an implantable delivery device.

Formulations for topical administration may include, but are not limited to, lotions, ointments, gels, creams, suppositories, drops, liquids, sprays and powders. Conventional pharmaceutical carriers, aqueous, powder or oily bases, thickeners and the like may be necessary or desirable.

Compositions for oral administration include powders or granules, suspensions or solutions in water or non-aqueous media, sachets, capsules or tablets. Thickeners, diluents, flavorings, dispersing aids, emulsifiers or binders may be desirable.

Formulations for parenteral administration may include, but are not limited to, sterile solutions, which may also contain buffers, diluents and other suitable additives.

Formulations for implantable delivery devices may similarly include, but are not limited to, sterile solutions, which may also contain buffers, diluents and other suitable additives.

Dosing is dependent on responsiveness of the condition for treatment, but will normally be one or more doses per day, with course of treatment lasting from several days to several months or until a required effect is achieved. Persons ordinarily skilled in the art can easily determine optimum dosages, dosing methodologies and repetition rates. Slow release administration regimes may be advantageous in some applications.

According to preferred embodiments of the present invention, providing the stem cells with the conditions for ex-vivo cell proliferation comprises providing the cells with nutrients and with cytokines. Preferably, the cytokines are early acting cytokines, such as, but not limited to, stem cell factor, FLT3 ligand, interleukin-1, interleukin-2, interleukin-3, interleukin-6, interleukin-10, interleukin-12, tumor necrosis factor-α and thrombopoietin. It will be appreciated in this respect that novel cytokines are continuously discovered, some of which may find uses in the methods of cell expansion of the present invention.

Late acting cytokines can also be used. These include, for example, granulocyte colony stimulating factor, granulocyte/macrophage colony stimulating factor, erythropoietin, FGF, EGF, NGF, VEGF, LIF, Hepatocyte growth factor and macrophage colony stimulating factor.

The stem cells to be expanded by the method of the present invention can be embryonic stem cells or adult stem cells. Embryonic stem cells and methods of their retrieval are well known in the art and are described, for example, in Trounson A O (Reprod Fertil Dev (2001) 13: 523), Roach M L (Methods Mol Biol (2002) 185: 1), and Smith A G (Annu Rev Cell Dev Biol (2001) 17:435). Adult stem cells are stem cells, which are derived from tissues of adults and are also well known in the art. Methods of isolating or enriching for adult stem cells are described in, for example, Miraglia, S. et al. (1997) Blood 90: 5013, Uchida, N. et al. (2000) Proc. Natl. Acad. Sci. USA 97: 14720, Simmons, P. J. et al. (1991) Blood 78: 55, Prockop D J (Cytotherapy (2001) 3: 393), Bohmer R M (Fetal Diagn Ther (2002) 17: 83) and Rowley S D et al. (Bone Marrow Transplant (1998) 21: 1253), Stem Cell Biology Daniel R. Marshak (Editor) Richard L. Gardner (Editor), Publisher: Cold Spring Harbor Laboratory Press, (2001) and Hematopoietic Stem Cell Transplantation. Anthony D. Ho (Editor) Richard Champlin (Editor), Publisher: Marcel Dekker (2000).

A presently preferred source for adult stem cells is the hematopoietic system. Hence, according to a preferred embodiment of the present invention the stem cells are hematopoietic stem cells. Such stem cells can be derived from bone marrow, peripheral blood and neonatal umbilical cord blood. Methods of enriching white blood cells (mononuclear cells) for stem cells are well known in the art, including, selecting for CD133 and CD34⁺ expressing cells. CD 133⁺ and CD34⁺ cells include pluripotent stem cells and very early progenitor cells, which, under the appropriate conditions may revert to stem cells, as they are not committed cells.

One most surprising result obtained while reducing the present invention to practice was that stem cells present in the mononuclear cell fraction of blood (i.e., white blood cells), can undergo expansion using the methods of the present invention in a fashion similar to stem cells enriched CD34⁺ cell fraction of blood. Hence, according to an embodiment of the present invention, the stem cells that undergo expansion are mixed (e.g., not separated from, not enriched) with committed cells. This embodiment of the present invention is of particular advantage because it relieves the tedious need for cell separation prior to ex-vivo culturing the cells.

In another embodiment, the cells are enriched for hematopoietic CD133⁺ cells or CD34⁺ cells and are characterized by an absence, or significantly diminished expression of cell surface antigens CD38 and Lineage specific antigens (Lin, including: CD3, CD61, CD19, CD33, CD14, CD15 and/or CD4).

It was experimentally found that reducing the capacity of the stem cells in responding to the disclosed signaling pathways is reversible, e.g., inherently reversible. In some experiments, following 16-18 weeks in culture the cells ceased to expand and started to differentiate. In other words, cells expanded using the protocols of the present invention to not transform into cell lines. Hence, by exposing such cells following sufficient expansion to growth conditions by which differentiation is induced, one would be able to direct the ex-vivo differentiation of the cells to desired direction, including ex vivo and in vivo cis- and trans-differentiation.

As used herein “cis-differentiation” refers to differentiation of adult stem cells into a tissue from which they were derived. For example, the differentiation of CD34⁺ hematopoietic cells to different committed/mature blood cells constitutes cis-differentiation.

As used herein “trans-differentiation” refers to differentiation of adult stem cells into a tissue from which they were not derived. For example, the differentiation of CD34⁺ hematopoietic cells to cells of different tissue origin, e.g., myocites constitutes trans-differentiation.

The stem cells used for cell expansion in context of the present invention can be obtained from any tissue of any multicellular organism including both animals and plants. Stem cells were shown to exist in many organs and tissues and are believed to exist in all tissues of animals, including, but not limited to, bone marrow (Rowley S D et al. (1998) Bone Marrow Transplant 21: 1253), peripheral blood (Koizumi K, (2000) Bone Marrow Transplant 26: 787, liver (Petersen B E et al. (1998) Hepatology 27: 433) and brain (Pagano S F et al. (2000) Stem Cells 18: 295). It is anticipated that all such cells are expandable using the methods of the present invention.

In a recent study (see PCTIL03/00235, to Peled, from which the present application claims priority) the present inventor unexpectedly discovered that ex vivo expanded stem cells differentiate into various cell type, including heart, lung, bone marrow and vascular cells following in vivo administration.

Depending on the source stem cells and target organ, differentiation can be either cis-differentiation or trans-differentiation or a combination of both.

As is mentioned hereinabove “cis-differentiation” refers to differentiation of stem cells into a tissue identical to the tissue from which they were derived. For example, the differentiation of CD34+ hematopoietic cells to different committed/mature blood cells constitutes cis-differentiation.

As is mentioned hereinabove “trans-differentiation” refers to differentiation of stem cells into a tissue distinct from which they were derived. For example, the differentiation of CD34+ hematopoietic cells to cells of different tissue origin, e.g., cardiac cells, constitutes trans-differentiation.

Since the expanded stem cells of the present invention are capable of differentiating in vivo into a variety of specific cell types, and since differentiation can be predetermined according to source and target tissue combinations, the method of the present invention can be utilized in cell replacement therapy.

Since transplantation of cord blood stem cells into MI rats have been shown to result in cell differentiation and homing of differentiated cells to loci of an MI scar and injured lung parenchyma, stem cells, expanded and administered using the methods described hereinabove, can be used to regenerate damaged tissue and in cell replacement therapy. Thus, the present methodology can be used in treating disorders which require cell or tissue replacement.

The disorder can be a neurological disorder, a muscular disorder, a cardiovascular disorder, an hematological disorder, a skin disorder, a liver disorder, and the like.

Myelin disorders form an important group of human neurological diseases that are, as yet, incurable. Progress in animal models, particularly in transplanting cells of the oligodendrocyte lineage, has resulted in significant focal re-myelination and physiological evidence of restoration of function (Repair of myelin disease: Strategies and progress in animal models. Molecular Medicine Today. 1997. pp. 554-561). Future therapies could involve both transplantation and promotion of endogenous repair, and the two approaches could be combined with ex vivo manipulation of donor tissue. Defects in cartilage and bones can also be treated using the teachings of the present invention. Methods of utilizing stem cells for treating such disorders are provided in U.S. Pat. No. 4,642,120. Skin regeneration of a wound or burn in an animal or human can also be treated using the teachings of the present invention. Methods of utilizing stem cells for treating such disorders are provided in U.S. Pat. No. 5,654,186 and U.S. Pat. No. 5,716,411.

In addition to the above-described application, the teachings of the present invention can also be utilized in several other therapeutic applications.

Transplantation of hematopoietic cells has become the treatment of choice for a variety of inherited or malignant diseases. While early transplantation procedures utilized the entire bone marrow (BM) population, recently, more defined populations, enriched for stem cells (CD34+ cells) have been used (Van Epps D E, et al. Harvesting, characterization, and culture of CD34+ cells from human bone marrow, peripheral blood, and cord blood. Blood Cells 20:411, 1994). In addition to bone marrow, such cells could also be derived from other sources such as peripheral blood (PB) and neonatal umbilical cord blood (CB) (Emerson S G. Ex-vivo expansion of hematopoietic precursors, progenitors, and stem cells: The next generation of cellular therapeutics. Blood 87:3082, 1996). Compared to BM, transplantation with PB cells shortens the period of pancytopenia and reduces the risks of infection and bleeding (Brugger W, et al. Reconstitution of hematopoiesis after high-dose chemotherapy by autologous progenitor cells generated in-vivo. N Engl J Med 333: 283, 1995; Williams S F, et al. Selection and expansion of peripheral blood CD34+ cells in autologous stem cell transplantation for breast cancer. Blood 87: 1687, 1996; Zimmerman R M, et al. Large-scale selection of CD34+ peripheral blood progenitors and expansion of neutrophil precursors for clinical applications. J Hematotherapy, 5: 247, 1996).

An additional advantage of using PB for transplantation is its accessibility, although to date the limiting factor in PB transplantation stems from the low number of circulating pluripotent stem/progenitor cells available for harvesting. To obtain enough PB-derived stem cells for transplantation, these cells are “harvested” by repeated leukophoresis following their mobilization from the marrow into the circulation by treatment with chemotherapy and cytokines. Such treatment is obviously not suitable for normal donors. Thus, the use of ex vivo expanded stem cells for transplantation provides several advantages: (i) it reduces the volume of blood required for reconstitution of an adult hematopoietic system and may obviate the need for mobilization and leukophoresis; (ii) it enables storage of small number of PB or CB stem cells for potential future use; and (iii) it traverses contamination limitations often associated with autologous transplantation of recipients with malignancies. In such cases, contaminating tumor cells in autologous infusion often contribute to the recurrence of the disease, selecting and expanding CD34+ stem cells will reduce the load of tumor cells in the final transplant.

In addition, expanded stem cell cultures are depleted of T lymphocytes, and thus are advantageous in allogeneic transplants in which T-cells contribute to graft-versus-host disease (Koller M R, Emerson S G, Palsson B O. Large-scale expansion of human stem and progenitor cells from bone marrow mononuclear cells in continuous perfusion cultures. Blood 82:378, 1993; Lebkowski J S, et al. Rapid isolation and serum-free expansion of human CD34+ cells. Blood Cells 20: 404, 1994).

Clinical studies indicate that transplantation of ex vivo expanded cells derived from a small number of PB CD34+ cells can restore hematopoiesis in recipients treated with high doses of chemotherapy, although the results do not yet allow firm conclusions about long term in vivo hematopoietic capabilities of these cultured cells.

For successful transplantation, shortening the duration of the cytopenic phase, as well as long-term engraftment, is crucial. Inclusion of intermediate and late progenitor cells in the transplant could accelerate the production of donor-derived mature cells thereby shortening the cytopenic phase.

It is thus important, in such applications that ex-vivo expanded cells include, in addition to stem cells, more differentiated progenitor cells in order to optimize short-term recovery and long-term restoration of hematopoiesis. Expansion of intermediate and late progenitor cells, especially those committed to the neutrophilic and megakaryocytic lineages, concomitant with expansion of stem cells, should serve this purpose (Sandstrom C E, et al. Effects of CD34+ cell selection and perfusion on ex vivo expansion of peripheral blood mononuclear cells. Blood 86: 958, 1995). Such cultures may be useful in restoring hematopoiesis in recipients with completely ablated bone marrow, as well as in providing a supportive measure for shortening recipient bone marrow recovery following conventional radio- or chemo-therapies. In addition to the above, the teachings of the present invention can also be applied towards hepatic regeneration, muscle regeneration, and stimulation of bone growth for applications in osteoporosis. The teachings of the present invention can also be applied to cases which require enhanced immune response or replacement of deficient functions, such as, for example, adoptive immunotherapy, including immunotherapy of various malignancies, immuno-deficiencies, viral and genetic diseases [Freedman A R, et al. Generation of T lymphocytes from bone marrow CD34+ cells in vitro. (1996). Nature Medicine. 2: 46; Heslop H E, et al. Long term restoration of immunity against Epstein-Barr virus infection by adoptive transfer of gene-modified virus-specific T lymphocytes. (1996) Nature Medicine, 2: 551; Protti M P, et al. Particulate naturally processed peptides prime a cytotoxic response against human melanoma in vitro. (1996). Cancer Res., 56: 1210].

Reducing the capacity of the stem cells in responding to PI 3-kinase signaling pathways is by ex-vivo culturing the stem cells in a presence of an effective amount of a modulator capable of downregulating PI 3-kinase activity and/or gene expression, preferably, for a time period of 0.1-50%, preferably, 0.1-25%, more preferably, 0.1-15%, of an entire ex-vivo culturing period of the stem cells or for the entire period. While reducing the present invention to practice, it was uncovered that an initial pulsed exposure to a PI 3-kinase activity inhibitor is sufficient to exert cell expansion after the inhibitor was removed from the culturing set up.

According to an additional aspect of the present invention, there is provided an assay of determining whether a specific modulator of PI 3-kinase activity or gene expression is capable of inhibiting differentiation of cells. The assay according to this aspect of the present invention comprises culturing a population of cells capable of differentiating, such as stem cells, (e.g. CD34⁺ hematopoietic cells), progenitor cells, or cells of a substantially non-differentiated cell line, such as, but not limited to, USP-1 and USP-3 (Sukoyan M A (2002) Braz J Med Biol Res, 35(5):535, C6, c2, Cr/A-3, DB1 and B6-26 (U.S. Pat. No. 6,190,910), and H9.1 and H9.2 (Odorico J. S. (2001) Stem Cells 19: 193) in the presence or absence of the modulator and monitoring changes in differentiation of the cells over time, e.g., a few weeks to a few months. Increased differentiation, as compared to non-treated cells, indicates a modulator of PI 3-kinase activity or gene expression incapable of inhibiting differentiation, whereas a lack or decrease in differentiation as compared to untreated cells indicates a modulator capable of inhibiting differentiation, which can be used effectively as a modulator of PI 3-kinase activity, for example, in the methods of the present invention disclosed herein.

Preferably, culturing the population of stem cells or cells of a substantially non-differentiated cell line is performed in a presence of an effective amount of a cytokine, preferably, an early acting cytokine or a combination of such cytokines, e.g., thrombopoietin (TPO), interleukin-6 (IL-6), an FLT-3 ligand and stem cell factor (SCF). This assay can be used, by one ordinarily skilled in the art, to determine which of the antagonists listed below is most efficient for the purpose of implementing the various methods, preparations and articles-of-manufacture of the present invention which are further described hereinafter. To determine most effective concentrations and exposure time for achieving optimal results with stem cells of different origins.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### RAR-antagonists and Their Use in ex-vivo Hematopoietic Cell Expansion

### Material and Experimental Methods

High-affinity Retinoic Acid Receptor Antagonist (RAR) Synthesis:

Synthesis of the RAR Antagonist 4-[[4-(4-ethylphenyl)-2,2-dimethyl-(2H)-thiochomen-6-yl)]-benzoic acid, (AGN 194310):

The RAR antagonist AGN194310 was synthesized according to the procedure described by Johnson (26), with some modification.

Synthesis of 3-(4-methoxyphenylthio)-3-methyl-butyric acid:

A heavy-walled screw-cap tube was charged with 3-methyl-2-butenoic acid (13.86 gm) 3,3-dimethylacrylic acid, (138.4 mmol), 4-methoxythiophenol (143.2 mmol), and piperidine (41.6 mmol) [Aldrich]. The mixture was heated to 105-110° C. for 32 hours, then cooled to room temperature. The reaction mixture was dissolved in ethyl acetate (EtOAc) (700 ml) with stirring, and the resulting solution was washed with 1M aqueous HCl (50 ml×2), water (50 ml), and saturated aqueous NaCl (50 ml). The organic solution was thereafter dried over NaSO₄. Concentration of this organic solution under reduced pressure afforded an oil and 2 days incubation at −20° C. yielded a crystalline solid. Forty ml of pentane were added to the solid, which was then crushed and filtered. The solid was washed on filter paper with pentane (20 ml, 2 times) to yield the product 3-(4-methoxyphenylthio)-3-methyl-butyric acid, as pale yellow crystals (31.4 grams, 94.4% yield, m.p. 62-64° C.), [¹H-NMR(CDCl₃): d7.5 (t, 2H, J=8 Hz), d6.9 (t, 2H, J=6.7 Hz), d3.9 (s, 3H, J=16.1 Hz), d2.6 (s, 2H), d1.3 (s, 6H)].

Synthesis of 3-(4-methoxyphenylthio)-3-methyl-butyryl chloride:

93.62 mmol oxalyl chloride in 10 ml benzene was added to a solution of 3-(4-methoxyphenylthio)-3-methyl-butyric acid in 100 ml of benzene at room temperature, for 30 minutes. During the addition of the oxalyl chloride, the solution turned yellow. After stirring the reaction mixture for 4 hours at room temperature, the reaction solution was cooled to 5° C. and washed with ice cold 5% aqueous NaOH (5 ml×6) (a large volume of gas was released during this procedure), followed by ice-cold water (15 ml×2) and finally saturated aqueous NaCl (15 ml). The organic solution was dried over NaSO₄ and concentrated under reduced pressure to give the acyl chloride product as a clear yellow oil. This material was used without further purification in the next step. [¹H-NMR (CDCl₃): d3.8 (s, 3H), d3.1 (s, 2H), d1.4 (s, 6H)].

Synthesis of 6methoxy-2,2-dimethyl-thiochroman-4-one:

A solution of Tin (IV) chloride in 30 ml dichloromethane was added dropwise to a solution of 3-(4-methoxyphenylthio)-3-methyl-butyryl chloride in 180 ml dichloromethane, at 0° C., yielding a dark red solution. After stirring the reaction mixture at 0° C. for 2 hours, the reaction was quenched by the slow addition of 115 ml water. The dark red reaction mixture became yellow.

The organic layer was washed with 1M aqueous HCl (50 ml), 5% aqueous NaOH (50 ml) and a saturated solution of NaCl (50 ml) and was thereafter dried over magnesium sulfate. The resulting organic solution was concentrated under reduced pressure, and distilled under vacuum (135-142° C., 0.6 mm/Hg) to obtain 6-methoxy-2,2-dimethyl-thiochroman-4-one as a residual pale-yellow oil (11 grams, 80.7%); [¹H-NMR (CDCl₃): d7.6 (s, 1H), d7.1 (s, 1H), d7.0 (s, 1H), d3.8 (s, 3H), d2.86 (s, 2H), d1.46 (s, 6H)].

Synthesis of 6hydroxy-2,2-dimethyl-thiochroman-4-one:

Boron tribromide (20 grams) in 80 ml dichloromethane was added over a 20 minute period to a solution of 6-methoxy-2,2-dimethyl-thiochroman-4-one in 50 ml dichloromethane. The reaction mixture was cooled to −23° C. and stirred for 5 hours, cooled to −78° C., then quenched by the slow addition of 50 ml water (0.5 hour). Following warming to room temperature, the colorless precipitate was filtered. After separation of the organic layer, the aqueous layer was extracted with 120 ml dichloromethane. The combined organic layers were washed with saturated aqueous NaHCO₃ (50 ml), water (50 ml) and saturated aqueous NaCl, then dried over MgSO₄. Removal of the organic solvent under reduced pressure gave a green solid (6 grams of crude product). This product was dissolved in 100 ml diethyl ether and the resulting solution was diluted with 300 ml petroleum ether. Overnight incubation at −15° C. yielded a crystalline product (2.3 grams, 41% yield, m.p. 122-126° C.). The filtrate was evaporated under vacuum, and the residue (3.42 grams) was dissolved in 30 ml diethyl ether. The ether solution was diluted with 150 ml petroleum ether and the resulting mixture was kept in a freezer at −20° C. overnight. Precipitation and filtration of the solution yielded 1.5 grams of the product 6-methoxy-2,2-dimethyl-thiochroman-4-one. This compound was re-precipitated by dissolution in 30 ml diethyl ether, then diluted with 20 ml petroleum ether. Incubation at 4° C. overnight, yielded 1 gram (80.7% yield, m.p. 135-142° C., 0.6 mm/Hg) of the green crystalline product, 6-hydroxy-2,2-dimethyl-thiochroman-4-one. [¹H-NMR (CDCl₃): d7.8 (s, 1H), d7.7 (s, 1H), d7.1 (s, 1H), d2.8 (s, 2H), d1.45 (s, 6H)].

Synthesis of 2,2-dimethyl-4-oxo-thiochroman-6-yl-trifluoro-methanesulfonate:

Trifluoromethanesulfonic anhydride was added to a stirred solution of 6-hydroxy-2,2-dimethyl-thiochroman-4-one in anhydrous pyridine. The mixture was stirred for 4 hours at 0° C., then stirred overnight at room temperature. Concentration under high vacuum yielded a residue that was treated with diethyl ether (75 ml). The ether solution was separated from the precipitate resulting from the formation of a salt between pyridine and trifluoromethanesulfonic acid. The ether solution was washed with water, then aqueous NaCl, and dried over MgSO₄. After removing the ether, the residue was crystallized. Traces of pyridine were removed under high vacuum. 0.7 gram of the crude product was obtained, and was further purified by column chromatography using 14 grams silica, and a solution of 200 ml petroleum ether:ethyl acetate (95:5) (using 15 ml eluent solution×13). After evaporation of the product fractions, 0.62 gram of 2,2-dimethyl-4-oxo-thiochroman-6-yl-trifluoro-methanesulfonate was obtained as colorless crystals (76.5% yield, m.p. 70-74° C.), [¹H-NMR (CDCl₃): d7.9 (s, 1H), d7.3 (s, 2H), d2.8 (s, 2H), d1.4 (s, 6H)].

Synthesis of 2,2-dimethyl-6-trimethylsilanyl-ethynyl-thiochroman-4-one:

A solution of 2,2-dimethyl-4-oxo-thiochroman-6-yl-trifluoro-methanesulfonate in triethylamine and dimethylformamide was sparged with argon for 10 minutes. Trimethylsilylacetylene and bis[triphenylphosphine] palladium(II) chloride were added to this solution. The reaction mixture was heated in a bath at 95-100° C. and maintained a reaction temperature of 88-90° C., for 5 hours. The reaction solution was cooled to room temperature, diluted with 200 ml water, and extracted with 100 ml ethyl acetate (60 ml×3). The resulting organic phase was washed with water (50 ml×2), and brine (50 ml). Finally, the organic solution was dried over MgSO₄, evaporated under reduced pressure, and the resulting residue was further purified by column chromatography using 42 grams silica, and an eluent system composed of 400 ml petroleum ether:ethyl acetate (97:3), yielding 2,2-dimethyl-6-trimethylsilanyl-ethynyl-thiochroman-4-one (1.82 grams, 76.4% yield, m.p. 67-70° C.); [¹H-NMR (CDCl₃): d7.8 (s, 1H), d7.3 (s, 2H), d2.8 (s, 2H), d1.4 (s, 6H), d0.23 (s, 9H)].

Synthesis of 6ethynyl-2,2,-dimethylthiochroman-4-one:

A solution of 2,2-dimethyl-6-trimethylsilanyl-ethynyl-thiochroman-4-one in methanol and potassium bicarbonate was stirred overnight at room temperature. The potassium carbonate was dissolved and the reaction was evaporated to a reduced volume of 30-40 ml, diluted with water (to an approximate volume of 70-100 ml), cooled in an ice-water bath and extracted with diethyl ether (60 ml×3). The combined organic layers were washed with 30 ml water and saturated aqueous NaCl (30 ml) and dried over MgSO₄. Removal of the solvent under reduced pressure afforded 6-ethynyl-2,2-dimethylthiochroman-4-one as an orange solid (1.3 gram, 97.7% yield, m.p. 63-66° C.) [¹H-NMR (CDCl₃): d7.8 (s, 1H), d7.3 (s, 2H), d3.0 (s, 1H), d 2.8 (s, 2H), d1.4 (s, 6H)].

Synthesis of ethyl 4-iodobenzoate:

A mixture of 4-iodobenzoic acid, 25 ml ethyl alcohol and 20 ml solution of dry HCl in ethyl alcohol was refluxed for 2 hours. The solid was dissolved after 1 hour of boiling. The reaction solution was cooled to room temperature and evaporated under vacuum to a volume of 10 ml. A lower organic layer formed with the chemical conversion of the acid to the ester. The resulting mixture was cooled in an ice bath. To this mixture 80 ml of diethyl ether, dry sodium hydrogen carbonate (1 gram) and 50 grams of ice were added. This solution was stirred, washed by dissolution of a saturated solution of sodium bicarbonate in 50 ml water and water, dried over sodium sulfate, and evaporated under vacuum, yielding ethyl 4-iodobenzoate as a liquid oil product (5.43 gram, 96.1% yield) [¹H-NMR (CDCl₃): d7.8 (s, 1H), d7.79 (s, 1H), 7.6 (s, 1H), d4.4 (d, 2H, J=7.1 Hz), d1.4 (s, 3H)].

Synthesis of ethyl 4-[(2,2-dimethyl-4-oxo-thiochroman-6-yl)ethynyl]-benzoate:

A solution of 6-ethynyl-2,2-dimethyl-thiochroman-4-one and ethyl 4-iodobenzoate in 80 ml triethylamine was purged with argon for 10 minutes. 0.7 gram Pd[PPh₃]₂Cl₂ and 0.19 gram CuI were added to this solution. The solution was sparged with argon for an additional 5 minutes, then stirred for 2 days at room temperature. The reaction mixture was filtered through a pad of celite with a diethyl ether wash. The filtrate was evaporated under reduced pressure. The solid residue was purified by column chromatography (40 grams silica, petroleum ether:ethyl acetate 95:5, 750 ml eluent solvent system) to yield ethyl 4-[(2,2-dimethyl-4-oxo-thiochroman-6-yl) ethynyl]-benzoate (1.26 gram, 56.5% yield, m.p. 102-104° C.). [¹H-NMR(CDCl₃): d8.275 (s, 2H), d7.6 (s, 3H), d7.5 (s, 1H), d7.2 (s, 1H), d4.3 (t, 2H, J=7), d2.8 (s, 2H), d1.48 (s, 3H)].

Synthesis of Ethyl 4-[(2,2-dimethyl-4-trifluoromethanesulfonyloxy)-(2H)-thiochromen-6-yl)ethynyl]benzoate:

A solution of sodium bis(trimethylsilyl)amide (0.6 M solution in toluene) and 10 ml of tetrahydrofuran was cooled to −78° C. and a solution of ethyl 4-[(2,2-Dimethyl-4-oxo-thiochroman-6-yl)ethynyl]benzoate in 10 ml tetrahydrofuran (THF) was slowly added. After 30 minutes, a solution of 2-[N,N-bis(trifluoromethanesulfonyl)amino]pyridine in 7 ml THF was added to the reaction mixture. After 5 minutes, the cooling bath was removed and the reaction solution was warmed to room temperature, stirred overnight and quenched by the addition of a saturated aqueous solution of NH₄Cl (20 ml). Two solvent layers were formed. The solution mixture was extracted with ethyl acetate (75 ml). The combined organic layers were washed with 5% aqueous NaOH (10 ml), water (15 ml×2), dried over MgSO₄, then concentrated under reduced pressure. The crude product (1.74 gram) was purified by column chromatography with 35 grams silica, and 2% ethyl acetate/petroleum ether (500 ml, 20×25 ml) eluent system. After evaporation of the combined eluted product fractions, ethyl 4-[(2,2-dimethyl-4-trifluoromethanesulfonyloxy)-(2H)-thiochromen-6-yl) ethynyl]benzoate (1.16 gram, 71% yield, m.p. 100-104° C.) was obtained, as a pale yellow solid. [¹H-NMR (CDCl₃): d8.2 (s, 2H), d7.6 (s, 3H), d7.5 (s, 1H), d7.2 (s, 1H), d6.0 (s, 1H), d4.4 (t, 6H, J=24 Hz)].

Synthesis of ethyl 4-[[4-(4-ethylphenyl)-2,2-dimethyl-[2H]-thiochromen-6-yl]-ethynyl]-benzoate:

7.25 ml of 1.7 M LiC(CH₃)₃ in pentane were added to a solution of p-bromo-ethyl-benzene (cooled to −78° C.) in 4 ml of THF. A solution of 658.7 mg zinc chloride in 8 ml THF was added, and the reaction mixture was warmed to room temperature, stirred for 40 minutes, then transferred to a second flask containing ethyl 4-[(2,2-Dimethyl-4-trifluoromethylsulfonyl)-(2H)-thiochromen-6-yl)ethynyl]benzoate and Pd(PPh₃)₄ in 8 ml THF. The resulting solution was heated to 50° C. for 2 hours, stirred at room temperature overnight, then quenched by addition of saturated aqueous NH₄Cl (10 ml) for 10 minutes. Two layers formed. The mixture was extracted with 75 ml ethyl acetate and the combined organic layers were washed with water (10 ml), and saturated NaCl. After drying the organic solution over MgSO₄, the solution was concentrated under reduced pressure, and purified by column chromatography using 24 grams silica, and a petroleum ether:ethyl acetate (95:5) eluent system (200 ml) yielding ethyl 4-[[4-(4-ethylphenyl)-2,2-dimethyl-[2H]-thiochromen-6-yl]-ethynyl]-benzoate

[¹H-NMR (CDCl₃): d8.2 (s, 2H), d7.6 (s, 2H), d7.4 (s, 2H), d7.2 (s, 1H), d7.1 (s, 2H), d7.0 (s, 2H), d6.0 (s, 1H), d4.4 (t, 2H, J=24 Hz), d2.8 (t, 2H, J=15 Hz), d1.6 (s, 6H), d1.4 (t, 3H, J=14 Hz)].

Synthesis of 4-[[4-(4-Ethylphenyl)-2,2-dimethyl-(2H)-thiochroman-6-yl]-ethynyl]benzoic acid:

Two ml of a 2 M solution of NaOH were added to a solution of ethyl 4[[4-(4-ethylphenyl)-2,2-dimethyl-[2H]-thiochromen-6-yl]-ethynyl]benzoate in THF and ethanol. The solution was heated to 40° C., stirred overnight, then cooled to room temperature. The reaction mixture was acidified with 1 N HCl (4 ml). At the beginning of the process, the reaction mixture formed a heterogeneous system. The mixture was extracted with ethyl acetate (25 ml×2). The combined organic layers were washed with 10 ml water, saturated aqueous NaCl, and dried with NaSO₄, and the solvent was removed under reduced pressure. The residual solid (0.31 gram) was recrystallized from acetonitrile (25 ml) to yield 4-[[4-(4-ethylphenyl)-2,2-dimethyl-(2H)-thiochroman-6-yl]-ethynyl]benzoic acid, (AGN194310) (0.236 gram, 70% ) as a colorless solid (m.p. 210-212° C.) [¹H-NMR (DMSO-d6): d8.2 (s, 2H), d7.8 (s, 2H), d7.6 (s, 2H), d7.4 (s, 2H), d7.2 (s, 2H), d7.0 (s, 1H), d6.0 (s, 1H), d2.6 (t, 2H, J=35 Hz), d1.6 (s, 6H), d1.4 (t, 3H, J=46 Hz)].

Mononuclear Cell Fraction Collection and Purification:

Human blood cells were obtained from umbilical cord blood from female patients following full-term, normal delivery (informed consent was obtained). Samples were collected and processed within 12 hours postpartum. Blood was mixed with 3% Gelatin (Sigma, St. Louis, Mo.), sedimented for 30 minutes to remove most red blood cells. The leukocyte-rich fraction was harvested and layered on a Ficoll-Hypaque gradient (1.077 gram/ml; Sigma), and centrifuged at 400 g for 30 minutes. The mononuclear cell fraction in the interface layer was collected, washed three times and resuspended in phosphate-buffered saline (PBS) solution (Biological Industries) containing 0.5% bovine serum albumin (BSA, Fraction V; Sigma).

Purification of CD34⁺ Cells from Mononuclear Cell Fractions:

To purify CD34⁺ mononuclear cells, the fraction was subjected to two cycles of immuno-magnetic separation using the MiniMACS® or Clinimax® CD34 Progenitor Cell Isolation Kit (Miltenyi Biotec, Auburn, Calif.) as per manufacturer's recommendations. The purity of the CD34⁺ population obtained ranged from 95% to 98% as was determined by flow cytometry (see below).

To further purify the CD34⁺ population into CD34⁺38⁻ or the CD34⁺ Lin⁻ sub-fractions, the purified CD34⁺ cells were further labeled for CD38 (Dako A/S, Glostrup, Denmark) or lineage antigens (BD Biosciences, Erermbodegem, Belgium). The negatively labeled fraction was measured and sorted by a FACS sorter.

For CD34⁻Lin⁻ purification, the CD34⁻ fraction was depleted from cells expressing lineage antigens using a negative selection column (StemCell Technologies, Vancouver, BC, Canada).

Ex-vivo Expansion of CD34^{+/-} Cell Populations:

CD34⁺ expressing purified cells above were cultured in 24-well Costar Cell Culture Clusters (Coming Inc., Corning, N.Y.) or culture bags (American Fluoroseal Corp), at a concentration of 10⁴ cells/ml in alpha medium (Biological Industries, Beit Haemek, Israel) supplemented with 10% fetal bovine serum (FBS, Biological Industries). The following human recombinant cytokines were added: Thrombopoietin (TPO), interleukin-6 (IL-6), FLT-3 ligand and stem cell factor (SCF), all at final concentrations of 50 ng/ml each, though occasionally IL-3, at a concentration of 20 ng/ml, was added either together or instead of SCF. For non-hematopoietic cell differentiation, FGF, EGF, NGF, VEGF, LIF or Hepatocyte growth factor (HGF) were used to supplement the growth medium, either alone or in various combinations. All cytokines used were purchased from Perpo Tech, Inc. (Rocky Hill, N.J.). The cultures were incubated at 37° C., 5% CO₂, in a humidified atmosphere.

Alternatively, whole mononuclear fraction cells (MNC) were isolated, cultured and supplemented with cytokines, as above.

At weekly intervals, cell cultures were toped and semi-depopulated and were supplemented with fresh medium, serum and cytokines or supplemented with fresh growth medium, alone. At predetermined time points, cells were harvested, stained with trypan blue, counted, and cell morphology was determined via the use of cytospin (Shandon, UK)-prepared smears stained with May-Grunwald/Giemsa solutions.

RAR Antagonist Supplementation of ex-vivo Hematopoietic Stem/Progenitor Cell Cultures:

CD34⁺ purified and whole MNC cultures were prepared and maintained as described above. AGN 194310 RAR antagonist was added to test cultures at concentrations ranging from 1×10⁻³−1×10⁻¹¹ M [or 410 μg/l to 4.1×10⁻⁵ μg/l]. The antagonist was added for a predetermined, limited period, for up to three weeks or continuously during the entire culture period.

Morphological Assessment:

Morphological characterization of the resulting culture populations was accomplished on aliquots of cells deposited on glass slides via cytospin (Cytocentrifuge, Shandon, Runcom, UK). Cells were fixed, stained with May-Grunwald/Giemsa stain and examined microscopically.

Surface Antigen Analysis:

Cells were harvested, washed with a PBS solution containing 1% bovine sera albumin (BSA) and 0.1% sodium azide (Sigma), and stained at 4° C. for 60 minutes with fluorescein isothiocyanate or phycoerythrin-conjugated antibodies (all from Immunoquality Products, the Netherlands). The cells were then washed with the same buffer and analyzed by FACS caliber or Facstarplus flow cytometers. Cells were passed at a rate of 1000 cells/second, using saline as the sheath fluid. A 488 nm argon laser beam served as the light source for excitation. Emission of ten thousand cells was measured using logarithmic amplification, and analyzed using CellQuest software. Negative control staining of cells was accomplished with mouse IgG-PE (Dako A/S Glostrup, Denmark) and mouse IgG-FITC (BD Biosciences, Erembodegem, Belgium).

Determination of CD34 and Other Hematopoietic Marker Expression:

CD34 surface expression on short and long-term cultures initiated either with purified CD34⁺ cells or the entire MNC fraction was determined as follows: CD34⁺ cells were positively reselected (Miltenyi kit) and counted. Purity was confirmed by subsequent FACS and cell morphology analysis.

Reselected CD34⁺ cell subsets were stained for the following combination of antigens: CD34PE/CD38FITC and CD34PE/38, 33, 14, 15, 3, 4, 61, 19 (Lin) FITC. The fraction positive for CD34 and negative for CD38 was defined as CD34⁺CD38⁻. The fraction positive for CD34 and negative for LIN was defined as CD34⁺Lin⁻ cell fraction.

Cell Population Calculations:

FACS analysis results are given as percentage values of cells. Absolute numbers of subsets are calculated from the absolute number of CD34⁺ cells.

Determination of baseline levels of CD34⁺/CD38− and CD34⁺/Lin⁻ cells was conducted as follows: CD34⁺ cells were purified from 3 thawed cord blood units and stained for the above markers. The mean of these experiments was considered as the baseline value.

Total cell counts, numbers of CD34⁺ cells and subsets, and CFU numbers are presented as cumulative numbers, with the assumption that the cultures had not been passaged; i.e., the number of cells per ml were multiplied by the number of passages performed.

Assaying Colony Forming Unit (CFU) Ability:

Cells were cloned in semi-solid, methylcellulose-containing medium supplemented with 2 IU/ml erythropoietin (Eprex, Cilag AG Int., Switzerland), stem cell factor and IL-3, both at 20 ng/ml, and G-CSF and GM-CSF, both at 10 ng/ml (all from Perpo Tech). Cultures were incubated for 14 days at 37° C., 5% CO₂ in a humidified atmosphere.

Determination of LTC-CFUc Values:

Briefly, the ability of the cultures to maintain self-renewal was measured by determination of the content of colony forming unit cells in the long and extended long-term cultures (LTC-CFUc), as described in the references hereinabove.

Experimental Results

RAR Antagonist Treatment of Enriched CD34⁺ Populations Alters Surface Differentiation Marker Expression Resulting in Large Numbers of Cells with a Less-differentiated Phenotype in Short-term Cultures:

In order to determine retinoid receptor antagonist effects on the ex-vivo expansion of stem cells, CD34⁺ cell enriched cultures were initiated in the presence of a combination of 4 cytokines with and without different concentrations of the retinoic acid receptor antagonist AGN 194310. Two weeks after the initial seeding, the percentage of cells bearing the CD34⁺ marker (considered to be mostly committed progenitor cells), as well as the percentage of cells bearing the markers CD34⁺/CD38⁻ and CD34⁺Lin⁻ (considered to represent the stem and early progenitor compartment) was ascertained by FACS analysis.

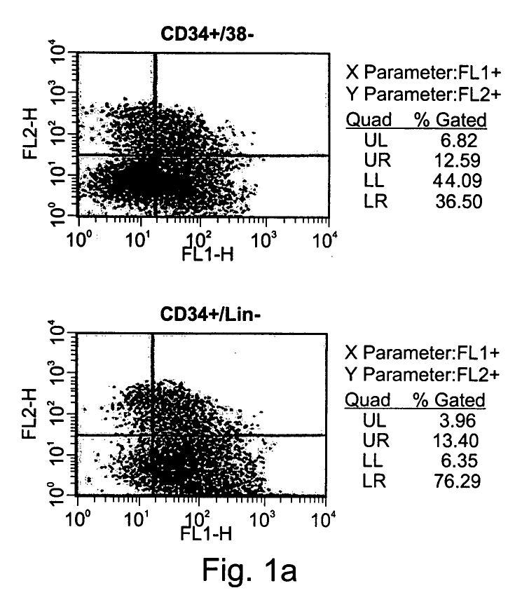

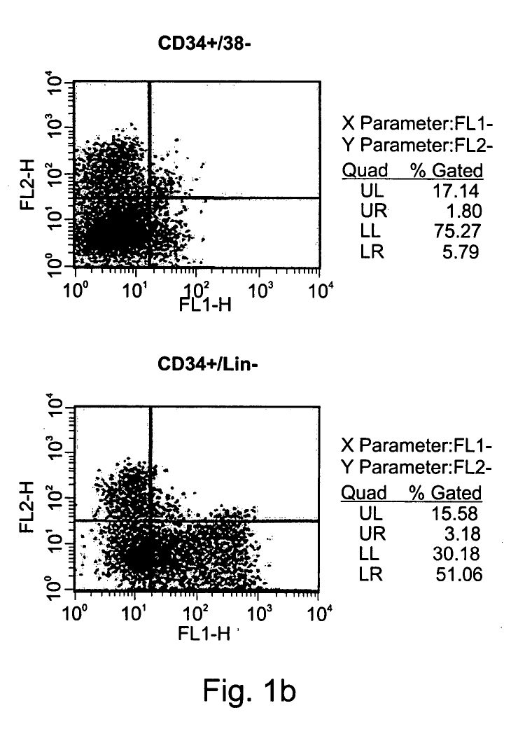

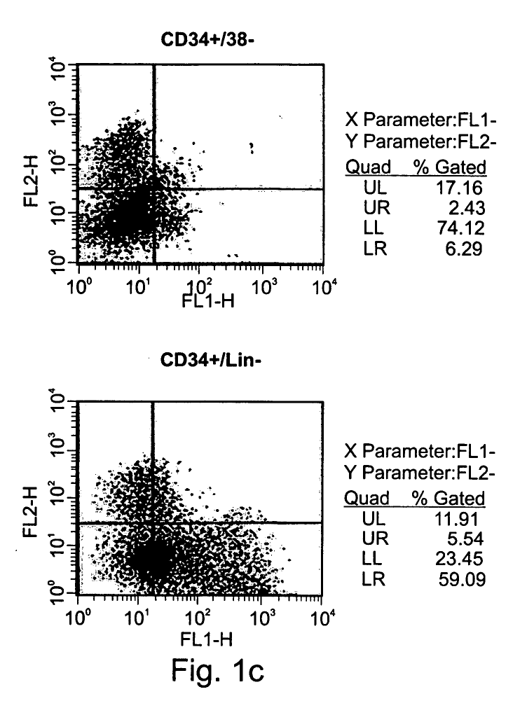

The FACS analysis plots are shown in FIGS. 1A-C. Retinoic acid receptor (RAR) antagonist treated cultures contained similar numbers of total and CD34⁺ cells as compared to cytokine-only treated cultures. RAR antagonist treatment completely abolished the expression of the CD38 antigen and concurrently, significantly inhibited the expression of the additional differentiation associated antigens CD33, CD14, CD15, CD4, CD3, CD19 and CD61, which was a totally unexpected phenomenon. Table 1 below summarizes the data from the FACS analysis.

TABLE 1

| | No. of cells | | | |
|---|---|---|---|---|
| | (× 10⁴) | % 34⁺cells | % 34⁺/38⁻cells | % 34⁺/Lin⁻cells |
| control | 52 | 19.41 | 6.82 | 3.96 |
| (cytokines | | | | |
| only) | | | | |
| RAR | 42 | 18.94 | 17.14 | 15.18 |
| antagonist, | | | | |
| 10⁻⁵ M | | | | |
| RAR | 52 | 19.59 | 17.16 | 11.91 |
| antagonist, | | | | |
| 10⁻⁶ M | | | | |

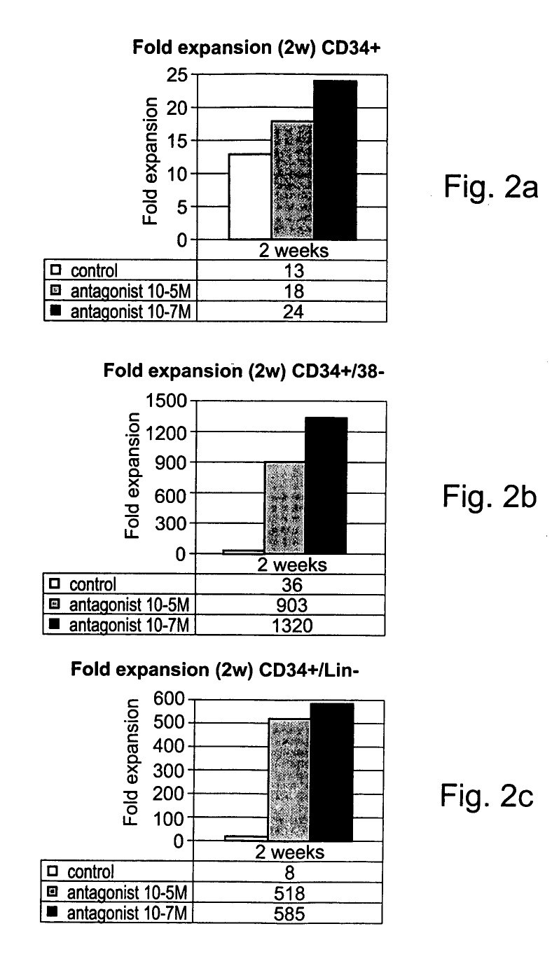

In an additional set of experiments, the stem and early progenitor cell subsets were measured following 2 weeks expansion from a re-selected CD34⁺ cell fraction. After two weeks in culture, CD34⁺ cells were re-selected and analyzed by FACS, as above, for the presence of the surface markers CD34⁺CD38⁻ and CD34⁺Lin⁻ (FIG. 2). RAR antagonist-treated cultures of reselected CD34⁺ cells revealed a 1000-fold increase in CD34⁺CD38⁻ and a 500-fold increase in CD34⁺Lin⁻ surface expression. In marked contrast, reselected control cultures treated with cytokines alone revealed only a 36-fold expansion of the CD34⁺CD38⁻ and an 8-fold expansion of the CD34⁺Lin⁻ compartments. Despite the marked differences in surface antigen expression, the total number of cells, and total number of CD34⁺ cells was comparable in all cultures. These results indicate that RAR antagonists preferably enable marked proliferation, yet limited differentiation of the stem cell compartment. RAR antagonists thus directly impact the high fold expansion of these rare cells during the short-term culture period. It could also be concluded that the antagonists do not have any positive or negative effect on more mature, committed CD34⁺ cells.

RAR Antagonist Treatment of Enriched CD34⁺ Populations Alters Surface Differentiation Marker Expression Resulting in Large Numbers of Cells with a Less-differentiated Phenotype in Long-term Cultures:

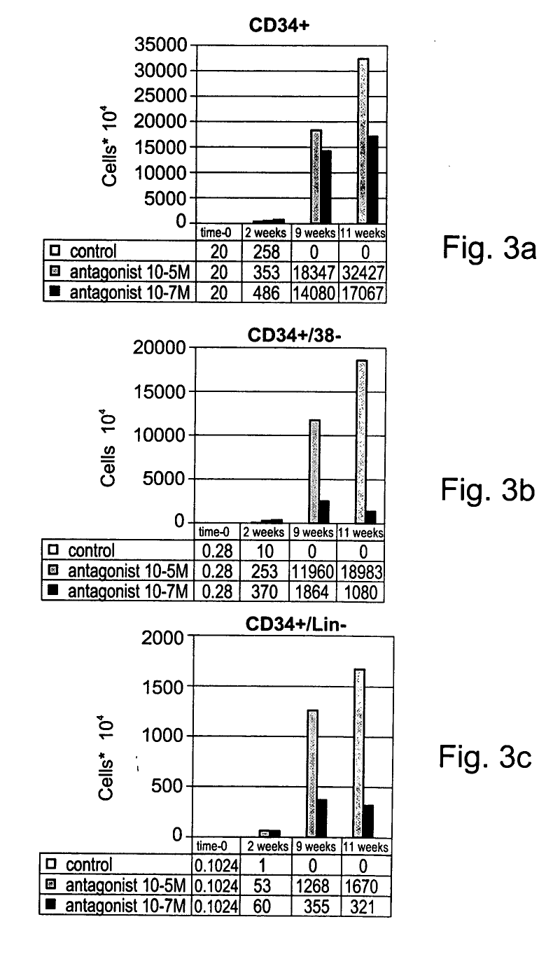

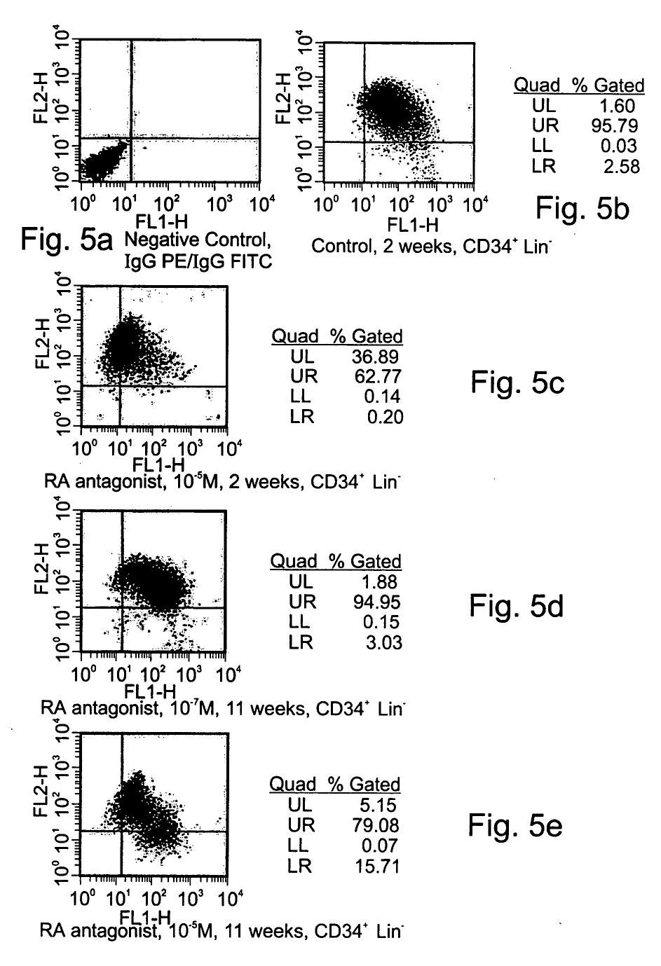

In order to find out whether the RAR antagonists potentiate a stem cell fraction with higher self-renewal ability, the effect of a limited, short-term (2-3 weeks) RAR antagonist culture treatment was tested on long-term expansion of CD34⁺ cells and subsets. Cultures were treated with RAR antagonists for the first three weeks only and then incubated for an additional eight weeks in the absence of the antagonist. In order to determine the effect of the antagonist on short and long term expansion of CD34+ cells, representative samples were taken from the cultures at the time intervals indicated (FIG. 3), for re-selection of CD34+ cells. CD34⁺ surface expression was again determined by FACS analysis following a positive selection step (FIG. 5B). During the first three weeks of incubation there were no significant differences between control and RAR antagonist treated cultures in terms of the numbers of CD34⁺-bearing cells. Following an additional eight weeks of incubation (week 11 of the culture), the RAR antagonist pre-treated cultures revealed a continuous, long-term increased expression of surface CD34+ antigen (FIG. 3A) whereas no CD34⁺ cells could be detected in the control cultures. A 92-fold increase in expression was seen in RAR antagonist treated cultures between week three to eleven and a 1621-fold expansion of this compartment occurred since the initiation of the cultures.

Expression of the CD34⁺CD38⁻ and CD34⁺Lin⁻ surface markers was verified in a highly purified, CD34+ re-selected fraction (FIGS. 3B-C). After two weeks in culture, while control samples revealed a modest 10-fold increase in CD34⁺Lin⁻ surface expression, RAR antagonist treated cultures expanded by a marked 530-fold. CD34⁺Lin⁻ expression at week eleven, 9 weeks after the termination of the treatment with the antagonist, revealed a 16,700-fold increase in CD34⁺Lin⁻expression. Comparison between the fold-expansion of RAR antagonist treated cultures versus that of control cells indicates that only the former enables a significant continuous proliferation of stem cells in extended long-term cultures. The continued expansion of stem cells in the absence of RAR antagonists indicates that even a relatively short pulse with the antagonist is sufficient to modify stem cell responses.

In an additional experiment, cultures were treated for one week only with cytokines only (control) or with cytokines and the RAR antagonist. A marked long-term effect of the RAR antagonist was noticed at week 13 of incubation, as is demonstrated in the results presented in Table 2 below. At week 20, the RAR antagonist pre-treated cultures deteriorated and the cells underwent normal differentiation, though in a slower kinetic that the control. These results indicate that a one-week RAR antagonist treatment is sufficient for dramatically modulating the proliferation ability of stem cells in ex-vivo conditions as the RAR antagonist transiently potentiate stem cell proliferation yet maintains their self-renewal ability.

TABLE 2

| Treatment | No. of CD34+ cells | No. of CFU*103 |
|---|---|---|
| Control (week 13) | 0 | 0 |
| Control (week 20) | 0 | 0 |
| RAR antagonist (10⁻⁵ M) | 10322 | 66355 |
| (week 13) | | |
| RAR antagonist (10⁻⁵ M) | 0 | 0 |
| (week 20) | | |

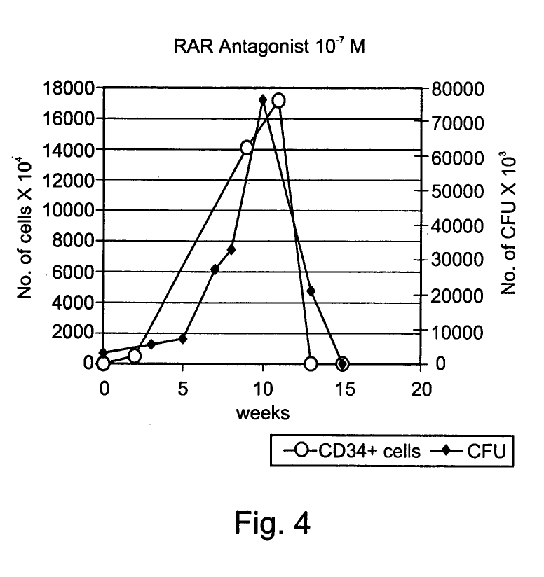

The limited extensive and durable cell proliferation enabled by the RAR antagonist is further demonstrated in another experiment, where it was shown that ex-vivo cultures supplemented with the RAR antagonist AGN194310 (10⁻⁷ M or 0.41 microgram/liter) enabled cell proliferation, only until 11 weeks post initial seeding of culture cells (FIG. 4). CFU forming ability was assayed as well, yet peak colony forming unit ability preceded peak absolute number of CD34⁺ cells by approximately one week, whereupon a precipitous decline in proliferation was evident, at which point cellular differentiation occurred, as evidenced by the loss of clonogenic (CFU forming ability) potential of the culture. These results, which describe a normal behavior of stem cells, namely extensive proliferation followed by differentiation are in marked contrast to previous reports that integration of a dominant negative retinoid receptor gene sustain infinite proliferation, in other words, resulted in the creation of cell lines (Muramatsu M, Biochem Biophys Res Commun 2001 Jul. 27:285(4):891-6 “reversible integration of the dominant negative retinoid receptor gene for ex vivo expansion of hematopoietic stem/progenitor cells), whereas in the present invention, cells were fully capable of normal differentiation, following extended ex-vivo proliferation.

A representative FACS chart plot of CD34⁺ cells 2 and 11 weeks following re-selection is shown in FIG. 5. While control cultures expressed markers for a more differentiated state, RAR antagonist treated samples expressed a less differentiated phenotype, as evidenced by the leftward shift in expression profile. These findings indicated that although not lineage negative, most of the CD34⁺ cells derived from RAR antagonist treated cultures expressed fewer lineage related surface markers.

RAR Antagonist Treatment of Mononuclear Cell Populations Expands a Population of Cells with a Less-differentiated Phenotype

Mononuclear cell fractions cultured in the presence of RAR antagonists and cytokines similarly revealed a significant increase in the number of CD34+Lin− cells (78%, 24%) as quantitated by FACS analysis from a reselected, highly purified CD34+ cell fraction, as compared to controls, 2 and 5 weeks (respectively), after initial seeding (Table 3). However, most remarkable is that these cells responded to the RAR antagonists and expanded an undifferentiated population, even in mixed culture conditions, without prior purification of the CD34⁺ population. RAR antagonist treatment was sufficient to stimulate specific expansion of the stem/progenitor cell compartment, as 5 weeks post seeding, while control MNCs had no detectable CD34⁺ population, RAR antagonist treated cultures revealed significant numbers of CD34⁺ cells, and those that were lineage marker deficient. Thus, any factors elaborated by the MNC culture cells that suppress CD34⁺ cell survival in control samples are insufficient to override the signal provided by the RAR antagonist to elaborate this compartment.

TABLE 3
Expansion of CD34⁺/Lin⁻ mononuclear cells

| | |
|---|---|
| Cytokines | Cytokines + RAR |
| only | antagonist 10⁻⁶ M |

2 weeks

| | | |
|---|---|---|
| No of CD34 cells × 10⁴* | 176 | 169 |
| No of CD34⁺/Lin⁻ × 10⁴* | 1.76 | 132.5 |
| % CD34/Lin⁻ | 1 | 78.4 |

5 weeks

| | | |
|---|---|---|
| No of CD34 cells × 10⁴* | 0 | 985 |
| No of CD34⁺/Lin⁻ × 10⁴* | 0 | 237.8 |
| % CD34/Lin⁻ | 0 | 24.1 |

*Cumulative value

RAR Antagonist Treatment Enhances Long-term Culture Colony Forming Unit (LTC-CFUc) Ability

Demonstration of a culture's ability to form colony forming units (CFUs) is another functional, in vitro method for verifying the presence of stem and early progenitor cells with a high self-renewal potential. Here it is demonstrate that culture pre-treatment with RAR antagonists enabled greater expansion of cells with a self-renewal capacity as evidenced by the presence of increasing numbers of CFU cells during the extended long-term culture period.

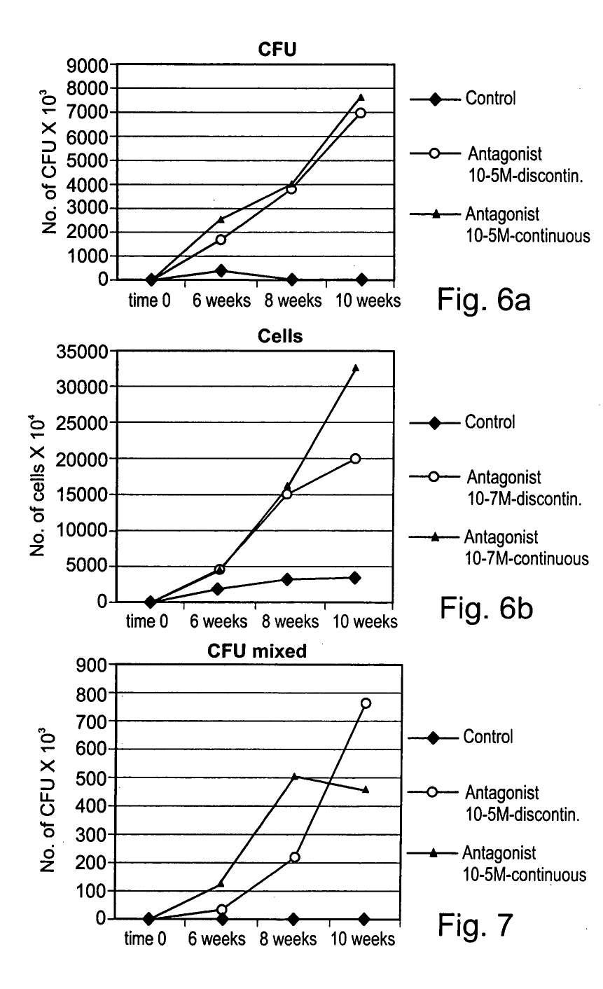

Long-term CD34⁺ cell cultures were supplemented with a combination of 4 cytokines, Flt3, TPO, IL-6 and IL-3, with and without varying predetermined concentrations of the RAR antagonist AGN 194310. RAR antagonist treatment of the cultures was for a limited period of three weeks or was continuous during the entire culture period. The ability to form CFUs was determined for long-term (6 weeks) cultures treated with 2 doses of the RAR antagonist for a short pulse or continuously and was compared to control samples treated with cytokines alone. Long-term cultures pulsed for the first 3 weeks with the antagonist revealed a 5-fold increase in CFU content as compared to control cultures (FIGS. 6A and 6B. Enumeration of mix-colonies indicated that control cultures did not contain any mix-colony forming unit cells, whereas antagonist treated cultures contained a higher number of cells with CFU-mix potential (FIG. 7).

RAR Antagonist Treatment Enhances Extended Long-term Culture Colony Forming Unit (LTC-CFUc) Ability:

The ability to form CFUc was determined for extended long-term (8-10 week) cultures treated with the RAR antagonists, as well. The differences in CFU content were significantly more pronounced during this culture period. RAR antagonist treatment markedly increased CFUc content between week 6 to 10, as compared to control cultures, which lost the ability to regenerate cells with CFU potential (FIGS. 6A and 6B) RAR antagonist pulse-treatment or continuous treatment increased CFU content by 15×10⁴. Pulse treatment with the antagonist yielded the highest level of CFU-mix content, as well (FIG. 7)

### Example 2

### RAR-antagonists and Their Use in ex-vivo Hepatocyte Expansion

### Material and Experimental Methods Isolation and Culture of Primary Hepatocytes:

Three intact livers were harvested from 3 week old VLVC female mice (Harlan Laboratories, Jerusalem, Israel), dissected and washed twice with DMEM (Beit Haemek, Israel), incubated with DMEM in the presence 0.05% collagenase for 30 minutes at 37° C., ground and passed through a 200 μm mesh sieve, yielding individual hepatocytes. Cells were washed twice and viability was ascertained with trypan blue. Cells were plated in collagen-coated, 35 mm tissue culture plates at a density of 4-×10⁴ live cells/ml in F12 media (containing 15 mM Hepes, 0.1% glucose, 10 mM sodium bicarbonate, 100 units/ml penicillin-streptomycin, glutamine, 0.5 units/ml insulin, 7.5 m cg/ml hydrocortisone, and 10% fetal bovine serum). Medium was changed after 12 hours, the cells were washed twice with phosphate buffered saline (PBS) and new medium was added. Medium was changed twice a week.

Hepatocytes were also grown in the presence of Epidermal Growth Factor (EGF), Platelet-Derived Growth Factor β chain (PDGF-BB), Fibroblast growth Factors (FGF-4) and Hepatocyte Growth Factor (HGF), at 20-50 ng/ml each, for the entire culturing period according to the method of Schwartz et al. (Schwartz R E, Reyes M, Koodie L, Jiang Y, Blackstad M, Lund T, Lenvik T, Johnson S, Hu W S, Verfaillie C M. Multipotent adult progenitor cells from bone marrow differentiate into functional hepatocyte-like cells. J Clin Invest. 2002; 109 (10): 1291-302). Hepatocytes were also grown in serum free medium according to the method of Runge et al. (Runge D, Runge D M, Jager D, Lubecki K A, Beer Stolz D, Karathanasis S, Kietzmann T, Strom S C, Jungermann K, Fleig W E, Michalopoulos G K. Serum-free, long-term cultures of human hepatocytes: maintenance of cell morphology, transcription factors, and liver-specific functions. Biochem Biophys Res Commun. 2000; 269(1): 46-53).

In all of the above-mentioned hepatocytes culture conditions, cells are grown in the presence or absence of the retinoic acid antagonist AGN 194310 at concentrations ranging from 10⁻⁵ M to 10⁻⁹ M.

After a period of 3 weeks, cultures treated with 10⁻⁵ M antagonist were detached with 0.25% trypsin, split and replated at a 1:2 ratio. The cells were either immunostained as described below, or visualized with Giemsa staining.

Murine hepatocyte cultures supplemented with EGF and HGF were evaluated as primary cultures, or following first and second passages. First passage cultures were grown for 2 weeks, split 1:2 and immunostained 8 days later for the presence of albumin, as described below. Second passage cultures were similarly grown for 2 weeks, split 1:2, and grown for an additional week, then split 1:4 and similarly immunostained 4 days later.

Histologic Characterization:

Hepatocytes and ex-vivo expanded cells were fixed in methanol directly in their cell culture plates and each procedure performed by standard procedures as outlined below.

The cellular uptake of organic anions by culture hepatocytes commonly use as markers of hepatocyte functionality, was studied by indocyanine green (ICG) dye uptake. ICG (Sigma, Jerusalem, Israel)) was dissolved in DMEM yielding a final concentration of 1 mg/ml (Yamada T, Yoshikawa M, Kanda S, Kato Y, Nakajima Y, Ishizaka S, Tsunoda Y. In vitro differentiation of embryonic stem cells into hepatocyte-like cells identified by cellular uptake of indocyanine green. Stem Cells. 2002; 20(2): 146-54). Ten days cultured hepatocytes were washed twice with PBS and incubated with 400 μl of the dye for 15 minutes at 37° C. Samples were then rinsed 3 times with PBS, and visualized by light microscopy.

Ex-vivo expanded cells and hepatocytes were stained with Giemsa stain, according to manufacturer's instructions (Shandon, Pittsburg, Pa.) for 4 minutes at room temperature, washed in buffer solution for 4 minutes and washed 3-4 times with rinse solution.

Immunocytochemistry

Hepatocytes were probed for expression of α-fetoprotein (AFP) using a rabbit polyclonal antibody raised against a recombinant protein of human origin that cross-reacts with AFP from mouse (H-140 Santa Cruz Technology, Santa Cruz, Calif.), and albumin using a rabbit antiserum to mouse albumin (Cappel-ICN, Aurora, Ohio). Cells were fixed in methanol at −20° C. for 10 minutes, rinsed with PBS for 5 minutes, and permeabilized with 0.1% triton-X (Sigma, Jerusalem Israel) in PBS for 5 minutes. The cells were then washed with Tris buffer saline (TBS) for 5 minutes and incubated with 1% bovine serum albumin (BSA) in PBS for 10 minutes. Endogeneous peroxidases were inactivated by incubation with peroxidase block (Envision, Dako, Carpinteria, Calif.) for 5 minutes, at room temperature. Cells were incubated with antibodies raised in rabbit against mouse albumin (at a dilution of 1:100); or against α-fetoprotein (at a dilution of 1:25) for 30 minutes. Samples were then visualized for peroxidase activity (via methods according to manufacturer's instructions using the Envision HRP-system (Dako, Carpinteria, Calif.), and counterstained with hematoxylin (Dako, Carpinteria, Calif.).

### Experimental Results

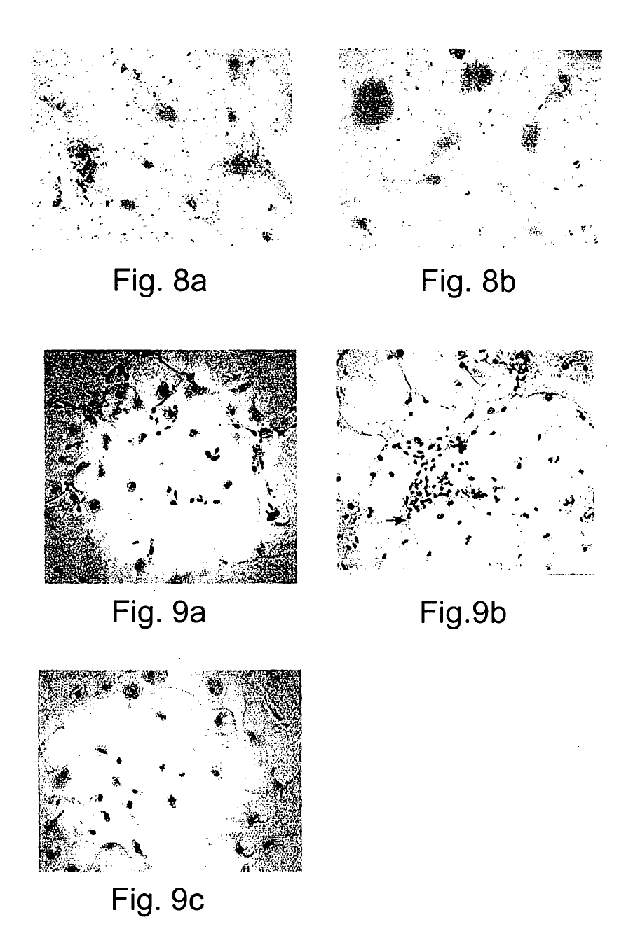

Primary cultures derived from 3 weeks old mouse livers, grown in media in the absence of cytokines, were probed for the expression of hepatocyte-specific markers including early development markers like α-fetoprotein (which is specific for less differentiated progenitor cells) and albumin which is a marker for mature hepatocytes, following 3 weeks in culture. Cultured cells stained positively (red-brown precipitate) for α-fetoprotein (FIG. 8A), and for albumin (data not shown) indicating the presence of functional hepatocytes. Incubation of the cultures in the presence of the 10⁻⁵ M retinoic acid antagonist resulted in an increase in the fraction of cells that stained positively for α-fetoprotein as compared to control cultures (FIG. 8B). This increase may signal the proliferation of early hepatocytes. Similarly, giemsa staining of the cultures revealed a large population of oval cells (hepatocyte stem progenitor cells are defined as oval cells) in cultures treated with the retinoic acid antagonist (FIG. 9B) while few were apparent in untreated control cultures (FIG. 9A).

Hepatocytes cultures grown in the presence of the antagonist and in the absence of cytokines for 3 weeks were trypsinized, split, and replated. The cells reattached to the culture plate and revealed typical hepatocytic morphology (FIG. 9C), as opposed to previous data indicating a difficulty in growing primary hepatocytes for extended periods of time in culture, especially in the absence of cytokines (Wick M, Koebe H G, Schildberg F W. New ways in hepatocyte cultures: Cell immobilization technique ALTEX. 1997; 14(2):51-56; Hino H, Tateno C, Sato H, Yamasaki C, Katayama S, Kohashi T, Aratani A, Asahara T, Dohi K, Yoshizato K. A long-term culture of human hepatocytes which show a high growth potential and express their differentiated phenotypes. Biochem Biophys Res Commun. 1999 Mar. 5;256(1):184-91; Tateno C, Yoshizato K. Long-term cultivation of adult rat hepatocytes that undergo multiple cell divisions and express normal parenchymal phenotypes. Am J Pathol. 1996; 148(2): 383-92).

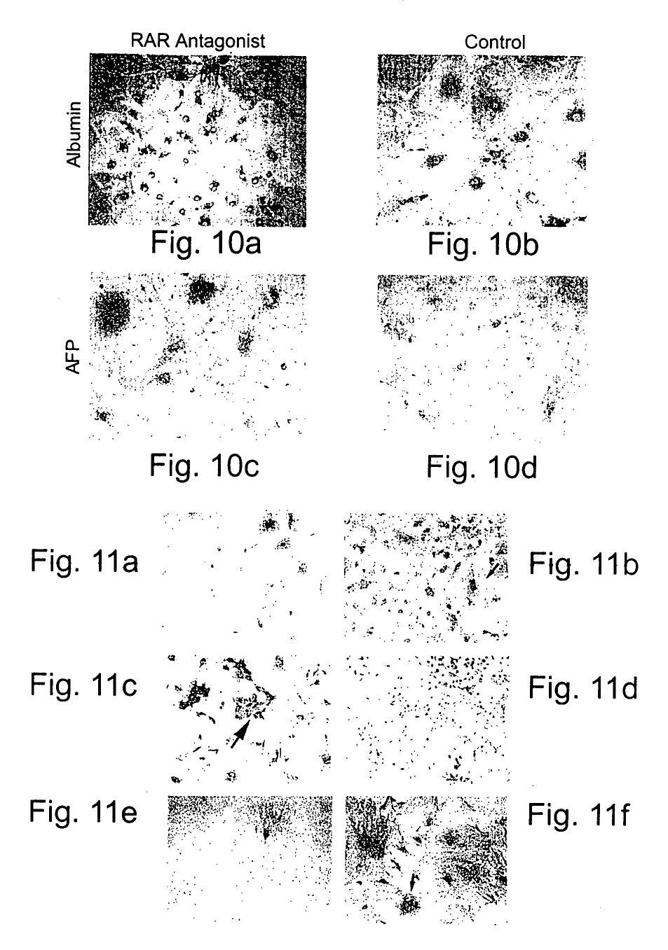

The supplementation of the culture media with growth factors in primary hepatocyte cultures treated with RAR antagonist revealed similar results to unsupplemented cultures, in that supplemented cultures stained positively for the production of α-fetoprotein (FIG. 10C), as compared to control cultures, supplemented with growth factors, but deprived of the RAR antagonist, where no immunostaining was evident (FIG. 10D). Background staining, as determined by probing for albumin expression, was negligible in RAR antagonist treated (FIG. 10A) and untreated, supplemented cultures (FIG. 10B). Thus culture supplementation with growth factors alone is insufficient to expand a less-differentiated cellular phenotype.

Similarly, first and second passages of growth factor-supplemented hepatocyte cultures were evaluated for their ability to persist in culture. In first passage growth factor-supplemented cultures both RAR antagonist treated (FIG. 11B) and untreated control cultures (FIG. 11A) revealed the presence of typical hepatocytes, however only RAR treated cultures (FIGS. 11C and D) revealed a large number of islets of oval cells, indicative of a hepatocyte stem cell population.

Second passage growth factor-supplemented cultures showed a marked diminishment in the number of hepatocytes evident in control cultures (FIG. 11E), as compared to RAR treated cultures (FIG. 11F), indicative of a failure of growth factor supplementation alone to provide expanded and persistent hepatocytes in culture. Only RAR antagonist treatment enabled expansion and long-term culture of hepatocyte populations.

### Example 3

### RXR and RAR+RXR Antagonists and Their Use in ex-vivo Cell Expansion

### Material and Experimental Methods

Synthesis of the RXR antagonist (2E, 4E, 6Z)-7-[3-propoxy-5,6,7,8-tetrahydro-5,5,8,8-tetramethyl-2-naphthalene-2-yl]-3-methylocta-2,4,6-trienoic acid] (LGN 100754):

The synthesis of LGN100754 was based on (i) Canan-Koch et al. J. Med. Chem. 39, 17, 3229-3234 [reaction scheme, page 3231; and (ii) Synthetic protocols from International Application No. PCT/US96/14876 (WO 97/12853) entitled Dimer-Selective RXR Modulators and Methods for Their Use. All materials were purchased from Ligand Pharmaceuticals Inc.

Synthesis of 6-ethynyl-1,1,4,4-tetramethyl-7-propoxy-1,2,3,4-tetrahydronaphthalene:

Phosphorus oxychloride (0.234 grams, 0.142 ml, 1.52 mmol) was added dropwise to dimethyl formamide (DMF) (4 ml) at room temperature under a nitrogen atmosphere. The solution was stirred for 30 minutes. The 1-(3-propoxy-5,6,7,8-tetrahydro-5,5,8,8,-tetramethylnaphthalen-2-yl) ethanone was added quickly (in one portion) to the orange solution, the reaction solution was heated to 60° C. and was stirred for 12 hours. The obtained dark brown solution was poured into ice water and the aqueous layer was adjusted to pH 7 with solid sodium hydrogen carbonate. Ethyl acetate extraction afforded the crude product, the chloroenal (6-[1-hydroxy,2-chloro-ethenyl]-1,1,4,4-tetramethyl-7-propoxy-1,2,3,4-tetrahydronaphthalene), 0.128 grams, as an orange/brown oil. A solution of the crude chloroenal in dioxane:water (3:2; 5 ml) was added to a solution of NaOH (0.061 grams, 1.52 mmol) in dioxane: H₂O (3:2; 20 ml), at 80° C., and the reaction mixture was stirred for 2 hours, to yield an orange reaction solution. The reaction solution was cooled to room temperature, poured into brine and extracted with EtOAc. The organic phase was dried (MgSO4), filtered, and concentrated to afford an orange oil which was purified by radial chromatography (10:1 hexane:ethyl acetate) to give the product 6-ethynyl-1,1,4,4,-tetramethyl-7-propoxy-1,2,3,4-tetrahydronaphthalene (39%) as a yellow oil [¹H-NMR (400 MHz, CDCl₃): d 7.38(s, 1H, Ar-H), 6.76(s,1H, Ar-H), 3.98 (t, J=6.6 Hz, 2H, OCH₃), 3.19 (s, 1H, CH),1.83 (m, 2H, CH₂),1.66 (m, 2H, 2CH₂),1.26 (s, 6H, 2CH₃),1.23 (s, 6H, 2CH₃), 0.93 (t, J=7.4 Hz, 3H, CH₃)].

Synthesis of 3-(3-propoxy-5,6,7,8-tetrahydro-5,5,8,8-tetramethylnaphthalene-2-yl) propynenitrile:

Ethyl magnesium bromide (3.33 ml of a 1.0 M solution in THF, 3.32 mmol) was added dropwise to a room temperature solution of the acetylene ether (6-ethynyl-1,1,4,4,-tetramethyl-7-propoxy-1,2,3,4-tetrahydronaphthalene) (0.450 grams, 1.66 mmol) in THF (10 ml). The solution was heated to reflux for 6 hours and then cooled to room temperature. Phenyl cyanate (0.40 grams, 0.50 ml, 3.33 mmol) was added (neat) to the reaction solution and the reflux was continued for additional 2 hours. The reaction solution was cooled to room temperature and quenched with a saturated ammonium chloride solution. Aqueous workup followed by radial chromatography (20:1hexanes:EtOAc) afforded the product 3-(5,5,8,8-tetramethyl-3-propoxy-5,6,7,8-tetrahydronaphthalen-2-yl)-propynenitrile (80%) as a yellow solid; ¹H-NMR (400 MHz, CDCl₃): d 7.44 (s, 1H, Ar-H), 6.78 (s, 1H, Ar-H), 3.97 (t, J=6.5 Hz, 2H, OCH₂), 1.83 (m, 2H, CH₂), 1.67 (m, 2H, 2CH₂), 1.27 (s, 6H, 2CH₃), 124 (s, 6H, 2CH₃), 1.03 (t, J=7.3 Hz, 3H, CH₃).

Synthesis of 3-(3-propoxy-5,5,8,8,-tetramethyl-5,6,7,8-tetrahydro-naphthalene-2-yl)but-2-enenitrile:

A flame dried flask was charged with a suspension of copper(I) iodide (0.057 grams, 0.298 mmol) in THF (5 ml) and the mixture was stirred at 0° C. under nitrogen atmosphere. Methyl lithium (0.43 ml of a 1.4 M solution in ether, 0.596 mmol) was added dropwise to give a colorless solution. The solution was cooled to −78° C. and afforded a yellow/brown color. The acetylene nitrile 3-(5,5,8,8-tetramethyl-3-propoxy-5,6,7,8-tetrahydronaphthalene-2-yl)propionitrile (0.040 grams, 0.135 mmol) in THF (3.0 ml) was added dropwise and the solution was stirred at −78° C. for 45 minutes and then quenched with methanol (5 ml). An aqueous workup afforded the cis-alkene nitrile 3-(3-propoxy-5,5,8,8,-tetramethyl-5,6,7,8-tetrahydro-naphthalene-2-yl)but-2-enenitrile (97%) as a yellow oil; ¹H-NMR (400 MHz, CDCl₃): d 7.19 (s, 1H, Ar-H), 6.78 (s, 1H, Ar-H), 5.35 (s, 1H, olefinic), 3.92 (t, J=6.4 Hz, 2H, OCH₂), 2.27 (s, 3H, CH₃), 1.79 (m, 2H, CH₂), 1.67 (s, 2H, 2CH₂), 1.28 (s, 6H, 2CH₃), 1.27 (s, 6H, 2CH₃), 1.02 (t, J=7.4 Hz, 3H, CH₃).

Synthesis of (2E, 4E, 6Z)-7-3[-propoxy-5,6,7,8-tetrahydro-5,5,8,8-tetramethyl-2-naphthalene-2-yl]-3-methylocta-2,4,6-trienoic acid]:

A round-bottomed flask equipped with N₂ bubbler, septa, and a stir bar was charged with a solution of 3-(3-propoxy-5,5,8,8,-tetramethyl-5,6,7,8-tetrahydro-naphthalene-2-yl)-but-2-enenitrile adduct in hexanes (5 ml) and toluene (5 ml), and was then cooled to −78° C. DIBAL (3.71 ml of a 1.0 M solution in toluene, 5.6 mmol) was added dropwise via syringe to the solution which was then stirred for 1.5 hour at −78° C., quenched with aqueous sodium potassium tartarate solution (10 ml) and warmed to room temperature over 30 minutes. The aqueous layer was acidified (1.0 M HCl to pH=4) and extracted with EtOAc (3×10 ml). The combined organic extracts were washed with water and brine, dried (sodium sulfate), filtered, and concentrated to give the cis-alkenyl, cis-3-(3-propoxy-5,5,8,8-tetramethyl-5,6,7,8-tetrahydro-naphthalene-2-yl)but-2-enal as a yellow oil; ¹H-NMR (400 MHz, CDCl₃): d 9.36 (d, J=8.4 Hz, 1H, CHO), 6.99 (s, 1H, Ar-H), 6.79 (s, 1H, Ar-H), 6.09 (s, J=8.4 Hz, 1H, olefinic), 3.90 (t, J=6.5 Hz, 2H, OCH₂),2.29 (s, 3H, CH₃),1.76 (m, 2H, CH₂), 1.68 (s, 2H, 2CH₂), 1.3 (s, 6H, 2CH₃), 1.24 (s, 6H, 2CH₃), 1.00 (t, J=7.4 Hz, 3H, CH₃).

A flame-dried round-bottomed flask equipped with a nitrogen bubbler, septa, and a stir bar was then charged with a solution of diethyl 3-ethoxycarbonyl-2-methyl-prop-2-enyl phosphonate (0.417 grams, 1.58 mmol, 0.39 ml) in THF (2.0 ml) and 1,3-Dimethyl-3,4,5,6-tetrahydro-2(1H)-pyrimidinone (DMPU, 0.7 ml). The solution was cooled to −78° C., and n-butyl lithium (0.96 ml of a 1.5 M solution in hexanes, 1.44 mmol) was added drop-wise via a syringe. The reaction mixture was warmed to 0° C. and stirred for 15 minutes. The resulting solution was then cooled to −78° C. and cis-3-(3-propoxy-5,5,8,8-tetramethyl-5,6,7,8-tetrahydro-naphthalene-2-yl)but-2-enal (1.31 mmol) was added dropwise via cannula. The solution was warmed to ambient temperature. After stirring for 1.5 hours, the reaction was quenched with water (15 ml), and the aqueous layer was extracted with EtOAc (3×10 ml). The combined organic layers were washed with aqueous CuSO₄, water, and brine, dried under sodium sulfate, filtered, and concentrated to give a crude ester (2E, 4E, 6Z)-7-3[-propoxy-5,6,7,8-tetrahydro 5,5,8,8-tetramethyl-2-naphthalene-2-yl]-3-methyl-octa-2,4,6-trienoic acid ethyl ester. The crude ester was hydrolyzed with KOH (excess) in methanol (7 ml) at reflux temperature and quenched with 1 M HCl (5 ml). The solution was concentrated, diluted with water (10 ml) and the aqueous layer was extracted with EtOAc (3×15 ml). The combined organic layers were washed with water and brine, dried over NaSO₄, filtered, concentrated, purified by radial chromatography followed by preparative silica gel TLC to give (2E, 4E, 6Z)-7-3[-propoxy-5,6,7,8-tetrahydro-5,5,8,8-tetramethyl-2-naphthalene-2-yl]-3-methylocta-2,4,6-trienoic acid] as a pale yellow solid; m.p. 177-179° C.; ¹H-NMR (400 MHz, CDCl₃): d 6.95 (s, 1H, Ar-H), 6.79 (s, 1H, Ar-H), 6.62 (dd, J=15.3, 11.0 Hz, 1H, olefinic), 6.22 (appp br d, 2H, 2* olefinic), 5.76 (s, 1H, olefinic), 3.89 (t, J=6.5 Hz, 2H, OCH₂), 2.19 (s, 3H, CH₃), 2.13 (s, 3H, 2CH₃, 1.77(m, 2H, CH₂), 1.68 (s, 4H, 2CH₂), 1.30 (s, 6H, 2CH₃), 1.23 (s, 6H, 2CH₃), 1.01 (t, J=7.4 Hz, 3H, CH₃).

Synthesis of the RAR+RXR antagonist 4-[5H-2,3-(2,5-Dimethyl-2,5-Hexano) 5-Methyl-8-nitrodibenzo [b,e][1,4]diazepin-11-yl) Benzoic acid [designated HX 531]:

Synthesis of the RAR+RXR antagonist HX531 was accomplished based on the procedure described by Masyuki Ebisawa et al., Chem. Pharm. Bull., 47(12): 1778-1786 (1999).

Synthesis of 2,5-Dimethyl-2,5-hexanediol:

Solutions of hydrogen peroxide (1.05 moles) and ferrous sulfate (1 mole and 1 mole of sulfuric acid) were added simultaneously and equivalently to an aqueous solution of t-butyl alcohol (285 ml or 3 moles in 800 ml of water containing 23 ml of sulfuric acid) at 30° C. A 36% yield of semi-solid product possessing a camphor-like odor was thereby isolated. The 2,5-dimethyl-2,5-hexanediol product was purified by drying and recrystallization (EtOAc) (melting point (mp): 85-87° C.).

Synthesis of 2,5-dichloro-2,5-dimethylhexane:

The synthesis was accomplished as previously described [Mayr, H., et al., Chem. Ber. 124: 203, 1999]. 2,5-Dimethyl-2,5-hexanediol (73.1 grams, 0.500 mol) was stirred with 37% aqueous HCl (250 ml) for 1 hour. The initially homogeneous mixture precipitated to yield a crystalline product. The product was extracted with 600 ml of petroleum ether and dried with CaCl₂. Evaporation of the solvent yielded 81.9 grams (89%) of an NMR-spectroscopically pure solid, which was recrystallized from petroleum ether (mp: 68-68.5° C.) as 2,5-dichloro-2,5-dimethylhexane.

Synthesis of 6-bromo-1,2,3,4-tetrahydro-1,1,4,4-tetramethylnaphthalene:

A 200 ml round-bottomed flask equipped with a stir bar and a reflux condenser was charged with a solution of bromobenzene (109 mmol, 17 ml) and 2,5-dichloro-2,5-dimethyl hexane (10 grams, 54.6 mmol) in dichloromethane (30 ml). Aluminum chloride (1.45 grams, 10.9 mmol) was added to the solution slowly, until spontaneous reflux subsides. After stirring for 10-15 minutes at room temperature, the reaction was poured into ice water (30 ml) and the layers were separated. The aqueous layer was extracted with EtOAc (5×20 ml). The combined organic layer was washed with water and brine, dried over sodium sulfate, filtered, and concentrated, to yield a 6-bromo-1,2,3,4-tetrahydro-1,1,4,4-tetramethylnaphthalene product.

A mixture of 6-bromo-1,2,3,4-tetrahydro-1,1,4,4-tetramethylnaphthalene (30 grams, 110 mmol), potassium carbonate (56.1 grams, 41 mmol) and copper iodide (4.53 grams) in o-xylene (300 ml) was heated at 150° C. for 14 hours. After removal of the solvent, the residue was purified by silica gel column chromatography (EtOAc:n-hexane 1:100) to yield the product 2-nitro-1-amino-[1,2,3,4-tetrahydro-1,1,4,4-tetramethylnaphthalene]-benzene as red plates (n-hexane) (36.09 grams, 82% yield of title product, mp: 118° C.].

A solution of 2-nitro-1-amino-[1,2,3.4-tetrahydro-1,1,4,4-tetramethylnaphthalene]-benzene (500 mg, 1.54 mmol) in DMF (10 ml) was added to a suspension of NaOH (60%, 92 mg, 2.31 mmol) in DMF (1 ml) and the mixture was stirred for 30 minutes, followed by addition of methyl iodide (0.5 ml) and additional stirring for 1 hour. After removal of the solvent, the residue was taken up in water, and was extracted with dichloromethane. The organic layer was washed with water and brine, and was dried over MgSO₄. Removal of the solvent under vacuum gave a crude product 2-nitro-1-methylamino-[1,2,3,4-tetrahydro-1,1,4,4-tetramethyl-naphthalene]benzene (543 mg).

2-Nitro-1-methylamino-[1,2,3,4-tetrahydro-1,1,4,4-tetramethyl-naphthalene]benzene (540 mg, 1.53 mmol) was dissolved in 20 ml of ethanol, and was hydrogenated over 10% ethyl alcohol (55 mg) for 1 hour. After filtration and removal of the solvent, the residue was chromatographed on silica gel (EtOAc:n-hexane 1:8) to give 2-amino-1-methylamino-[1,2,3,4-tetrahydro-1,1,4,4-tetramethyl-naphthalene]benzene as the product.

Terephthalic acid monomethyl ester chloride (381 mg, 1.91 mmol) was added to a solution of 2-amino-1-methylamino-[1,2,3,4-tetrahydro-1,1,4,4-tetramethyl-naphthalene]benzene (420 mg, 1.3 mmol) in benzene (10 ml) and pyridine (2 ml). The mixture was stirred for 4 hours, then poured into 2N hydrochloric acid, and extracted with EtOAc. The organic layer was dried and was then purified over silica-gel (EtOAc:n-hexane 1:8) to give the product 2-[amido-4-benzoic acid methyl-ester]-1-methyl-amino[1,2,3,4-tetrahydro-1,1,4,4-tetramethyl-naphthalene]-Benzene (631 mg).

A solution of 2-[amido-4-benzoic acid methyl-ester]-1-methyl-amino[1,2,3,4-tetrahydro-1,1,4,4-tetramethyl-naphthalene]-Benzene (630 mg, 1.30 mmol) in dichloromethane was added to polyphosphoric acid (6.0 grams) and the mixture was heated at 110° C. for 18 hours. After cooling, water was added to the reaction and the product was extracted with dichloromethane. The organic layer was washed with brine, dried over magnesium sulfate, and evaporated. The residue was purified by silica-gel column chromatography (EtOAc:n-hexane 1:6) to yield the product 2-[amido-4-benzoic acid methyl ester]-1-methylamino[1,2,3,4-tetrahydro1,1,4,4-tetramethylnaphthalene]4-nitrobenzene (104 grams).

KNO₃ (73 mg, 0.72 mmol) was added to a solution of 2-[amido-4-benzoic acid methyl ester]-1-methylamino[1,2,3,4-tetrahydrol, 1,4,4-tetramethylnaphthalene]4-nitrobenzene (200 mg, 0.44 mmol) in sulfuric acid (12 ml) at 0° C. After 2.5 hours, the mixture was poured into ice water and extracted with dichloromethane. The organic layer was washed successively with 1N NaHCO₃, water and brine, and dried over MgSO₄. After evaporation, the residue was purified by silica gel column chromatography (EtOAc:n-hexane 1:8) to give methyl 4-(5H-2,3-(2,5-dimethyl-2,5-hexano)-5-methyl-8-nitrodibenzo[b,e][1,4]diazepin-11-yl) benzoate (100 mg, 45.5%) and the product recovered (84 mg). This compound was hydrolyzed under basic conditions (2N NaOH/EtOH) as follows:

Synthesis of 4-(5H-2,3-(2,5-dimethyl-2,5-hexano)-5-methyl-8-nitrodibenzo[b,e][1,4]diazepin-11-yl) benzoic acid:

A solution of (5H-2,3-(2,5-dimethyl-2,5-hexano)-5-methyl-8-nitrobenzo[b,e][1,4]diazepin-11-yl]benzoic acid methyl ester (84 mg) in ethanol (4 ml) and 2N NaOH (2 ml) was stirred at room temperature for 2 hours. The mixture was poured into 2N hydrochloric acid, and extracted with dichloromethane. The organic layer was washed with brine, and dried over magnesium sulfate. After evaporation, the crude product was purified by silica gel column chromatography (dichloromethane:methanol 20:1, then 8:1) to give the product 4-(5H-2,3-(2,5-dimethyl-2,5-hexano)-5-methyl-8-nitrodibenzo[b,e][1,4]diazepin-11-yl) benzoic acid, or HX531.

RXR, RAR and RAR+RXR Antagonists Supplementation of ex-vivo Hematopoietic Stem/Progenitor Cell Cultures:

Cultures were prepared and maintained as described above. RXR, RAR, or RAR+RXR antagonists were added to some cultures at concentrations ranging from 10⁻⁴ M to 10⁻⁹ M (100 μM to 10⁻³ M] concentrations corresponding to diluting concentrations of 1550 μg/l to 0.155 μg/l]. The antagonist was added for a predetermined, limited time period, for up to three weeks or continuously during the entire culture period.

All other procedures including mononuclear cell fraction collection and purification, purification of CD34⁺ cells from mononuclear cell fractions, ex-vivo expansion of CD34^{+/-} populations, morphological assessment, surface antigen analysis, determination of CD34 and other hematopoietic marker expression and cell population calculations were carried out as described in the experimental methods section of Example 1 above.

### Experimental Results

Comparative Effects of RAR, RXR and RAR+RXR Antagonists and a Combination of RAR and RXR Antagonists on the ex vivo Expansion of Stem and Progenitor Cells in Culture:

CD34⁺ cell enriched cultures were initiated in the presence of a combination of 4 cytokines (TPO, FLT3, IL-6 and IL-3), with and without different concentrations of the following antagonists: (i) a retinoic acid receptor (RAR) antagonist AGN 194310, (ii) a retinoic X receptor (RXR) antagonist LGD 100754 and (iii) a combination of the RAR antagonist AGN 194310 and the RXR antagonist LGD 100754. Three and five weeks after the initial seeding, the percentage of cells bearing the CD34⁺ marker (considered to be mostly committed progenitor cells), as well as the percentage of cells bearing the markers CD34⁺/CD38⁻ and CD34⁺Lin⁻ (considered to represent the stem and early progenitor compartment) were ascertained by FACS analysis.

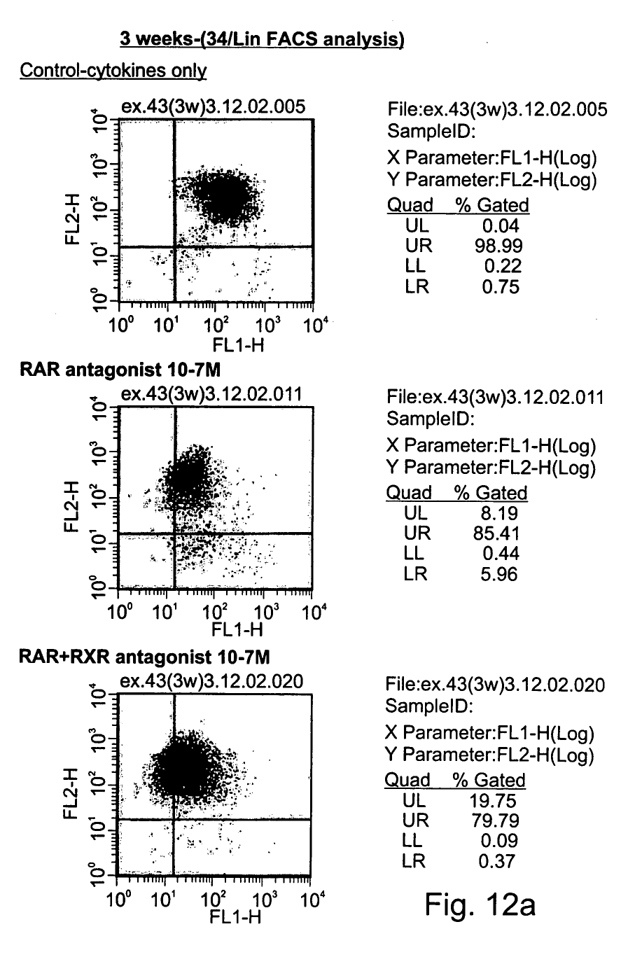

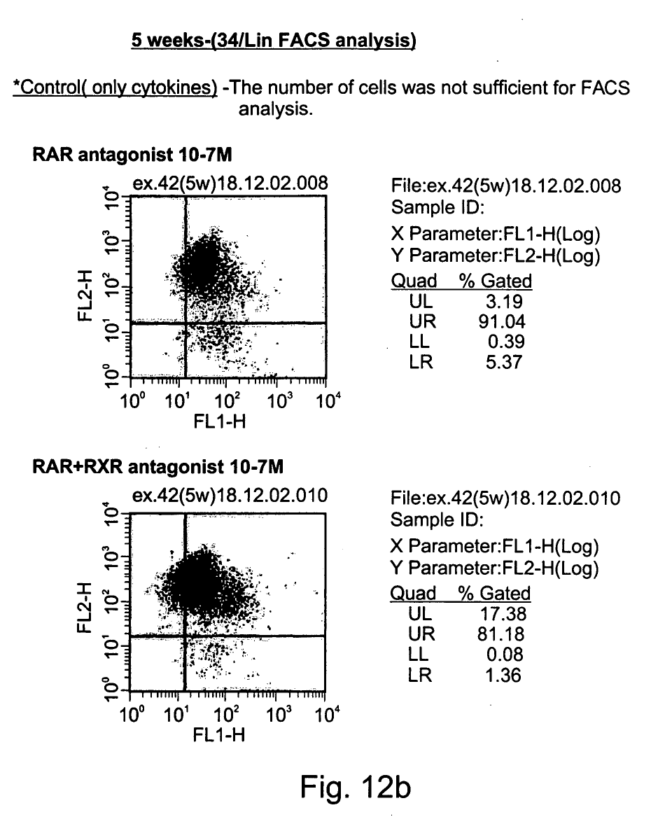

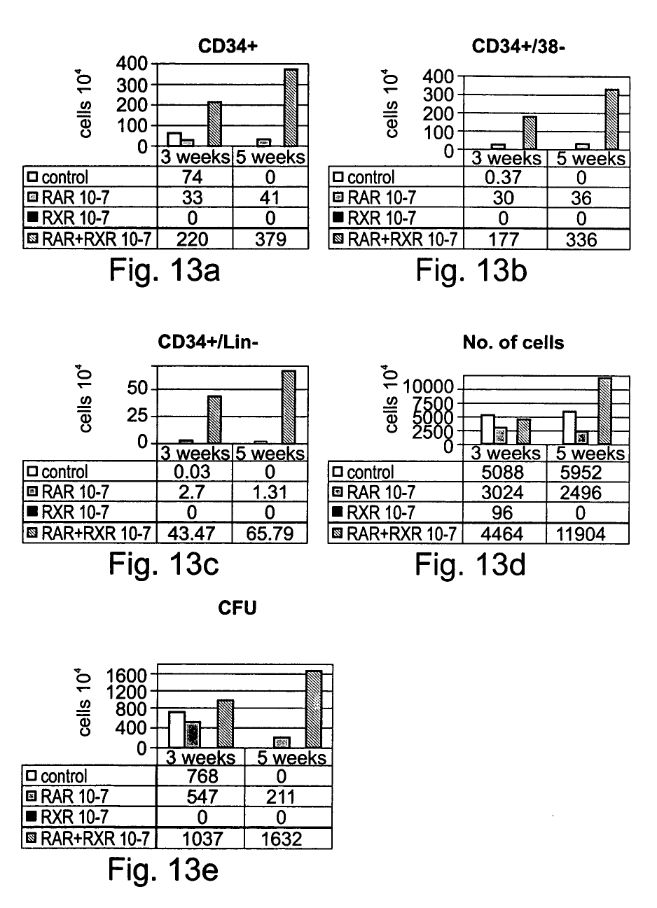

The data obtained from cell population counts, CFU counts and FACS analyses are illustrated in FIGS. 12a-b and 13a-e. The results show that while the RXR antagonist has no activity and the RAR antagonist exerts moderate activity when supplemented to the culture media at a concentration of 10⁻⁷ M and along with the cytokine IL-3 (cell-differentiation accelerator), treatment with the combination of RAR and RXR antagonists resulted in substantially higher levels of CFU, CD34⁺ cells, CD34⁺/38⁻ cells, and CD34⁺/Lin⁻ cells, as compared with the control (cytokines only), the RAR antagonist treatment, and the RXR antagonist treatment. Clearly, the combination of RAR and RXR antagonists exerts a synergistic effect on the ex vivo expansion of stem/progenitor cells.

In an additional experiment, CD34⁺ cell enriched cultures were initiated in the presence of a combination of 4 cytokines (TPO, FLT3, IL-6 and IL-3), with and without the RAR+RXR antagonist HX-531 (i.e., antagonist to both retinoic acid and retinoic X receptors) (10⁻⁶ M; MW=483). The levels of CFU and CD34⁺ cells were determined 3, 7, 9 and 11 weeks after the initial seeding. The results of this experiment are summarized in Table 4 below.

TABLE 4

| | | |
|---|---|---|
| | | RAR + RXR |
| | | ANTAGONIST |
| | CONTROL | HX-531 |
| Time | (cytokines only) | (10⁻⁵M) |

| after seeding | CFU | CD34⁺ | CFU | CD34⁺ |
|---|---|---|---|---|
| (weeks) | (×10³) | (×10⁴) | (×10³) | (×10⁴) |
| 3 | 2256 | 181 | 1920(120 | 167 |
| | | | mixed) | |
| 5 | 1338 | 46 | 8542 | 1636 |
| 9 | 307 | 0 | 36557 | 4977 |
| 11 | 0 | 0 | 67338 | 4055 |

These results indicate that the RAR+RXR antagonist preferably enables marked proliferation, yet limited differentiation of the stem cell compartment, thus directly impact the high fold expansion of stem/progenitor cells during short- and long-term culture period.

### Example 4

Synthesis of the Vitamin D Receptor (VDR) Antagonist 1 Alpha, 25-(OH) 2D3-26,23-lactone:

Synthesis of the four diastereoisomers of 1 alpha, 25-(OH) 2D3-26,23-lactone can be accomplished as described in Ishizuka, S. et. al, Archives of Biochemistry and Biophysics 242: 82,1985, or according to the following procedure:

Synthesis of Methyl 4-Iodo-2-Methyl-Butyrate:

To a suspension of lithium in 2 ml ether (dry) under stream of nitrogen, a solution of phenyl bromide in 3 ml ether was added dropwise. The reaction mixture was heated until complete dissolution of the lithium was achieved. A solution of methylene iodide in ether was prepared under a stream of argon and was cooled to −78° C. The phenyl lithium solution was added dropwise to this solution by a syringe during 0.5 hour, and a solution of methyl (R)-(+)-3-bromo-2-methylpropionate in ether (5 ml) was then added thereto. The reaction mixture was stirred overnight at 25° C. DMSO (7 ml) was then added and the ether was evaporated. The reaction mixture was stirred overnight at 100° C.

Synthesis of (1 alpha, 3 Beta, 5E, 7E, 20R, 1′E)-1,3-bis-(tert-butyldimethylsilyloxy)-20-Methyl(2-Methyl,1′-Heptenylate)-9,10-secopregna-5,7,10,(19)-triene:

To a suspension of lithium metal in 2 ml of dry ether, a solution of phenyl bromide in 3 ml of dry ether was added dropwise, under nitrogen atmosphere. An exothermic reaction was observed during the dissolution of the lithium metal. The reaction mixture was heated until complete dissolution of the lithium metal was achieved.

Triphenylphosphine 99% (1.447 grams, 5.52 mmol) and DMSO were added to the reaction solution of methyl 4-iodo-2-methyl-butyrate described above and the resulting mixture was heated to 100° C. for 18 hours. The mixture was then cooled to −30° C. under nitrogen atmosphere, and the phenyl lithium solution in ether was added thereto.

This reaction mixture was stirred at 0° C. for 1 hour and thereafter a hexane solution of the aldehyde CLP-8—Beta,5E,7E,20R,1′E)-1,3-bis-(tert-butyldimethylsilyloxy)-9,10-secopregna-5,7,10,(19)-triene-aldehyde—was added. The obtained mixture was stirred at 100° C. overnight. The ether and the hexane were thereafter distilled, the reaction mixture was cooled to 60° C. and 50 ml ethyl acetate in 75 ml water were added thereto. The Organic layer was separated, washed with 25 ml water and brine and dried over sodium sulfate. The organic solvent was evaporated under reduced pressure and the residue was dried under high vacuum and was purified on silica gel column (60 grams) with a mixture of hexane-EtOAc (98:2) as an eluent, to obtain 60 mg of the product (1 alpha,3 Beta,5E,7E,20R,1′E)-1,3-bis-(tert-butyldimethylsilyloxy)-20-(2-methyl,1′heptenylate)-9,10-secopregna-5,7,10,(19)-triene.

Synthesis of (1 alpha, 3 beta, 5E, 7E, 20R, 1′E)-1,3-bis-(tert-butyldimethylsilyloxy)-20-(2-methyl-2-hydroxy-1′heptenoic acid)-9,10-secopregna-5,7,10,(19)-triene:

(1 alpha, 3 Beta, 5E, 7E, 20R, 1′E)-1,3-bis-(tert-butyldimethylsilyloxy)-20-(2-methyl-1′-heptenylate)-9,10-secopregna-5,7,10,(19)-triene (60 mg) was dissolved in 3 ml THF and the solution was cooled to −78° C. under a stream of argon. LiN(iPr)2 was added to the reaction mixture, so as to obtain the lithium derivative, which was further reacted with oxygen for 1 hour at −78° C. Triphenylphosphine was then added and the reaction mixture was stirred for 30 minutes. The resulting reaction mixture was then evaporated under vacuum. A solution of KOH in methanol was added to the residue and the reaction mixture was heated to 60° C. for 2.5 hours and was thereafter diluted with 0.5 ml 1N HCl, and evaporated under vacuum. The residue was dissolved in chloroform and the product was purified on silica gel plate (20×20), using a mixture of 97:3 hexane-ethyl acetate (2 times) as the eluent, to obtain 6.3 mg of the product as fraction 2 (Rf=0.81).

The obtained product was then treated with a solution of 15.2 mg iodine in 2 ml methylene chloride, in the presence of pyridine (12 mg) and the reaction mixture was evaporated under vacuum and thereafter under high vacuum. The residue was dissolved with THF and n-Bu₃SnH (29.1 mg) was added thereto. The reaction mixture was stirred at room temperature for 4 hours and was thereafter evaporated under vacuum.

The residue was treated with catalytic amounts of HCl in methanol at 50° C. for 5 hours. The reaction mixture was evaporated under vacuum and the residue was purified on silica gel TLC plate (20×20) using a mixture of 95:5 chloroform-methanol as the eluent, to obtain 2.64 mg of the desired product 9,10-secocholesta-5,7,10(19)-trien-26-oic acid, 1,3,23,25-tetrahydroxy-gamma-lactone or (23S, 25R)-1alpha,25-DihydroxyvitaminD3-26,23-lactone, as fraction 1 (Rf=0.4); FAB-MS: Calc. 426.60, Found 426.88.

### Example 5

### Effect of Nicotinamide on ex-vivo Exansion of Hematopoietic Stem/Progenitor Cells

Nicotinamide Supplementation of ex-vivo Hematopoietic Stem/Progenitor Cell Cultures:

Cultures were prepared and maintained as described above. Nicotinamide was added to cell cultures at concentrations of 1, 5 or 10 mM for up to three weeks culture period. All other procedures including mononuclear cell fraction collection and purification, purification of CD34⁺ cells from mononuclear cell fractions, ex-vivo expansion of stem/progenitor cell populations, morphological assessment, surface antigen analysis, determination of CD34, CD38, Lin and other hematopoietic marker expression and cell population calculations were carried out as described in the experimental methods section of Example 1 above.

### Experimental Results

Effects of Nicotinamide on the ex-vivo Expansion of Stem and Progenitor Hematopoietic Cells:

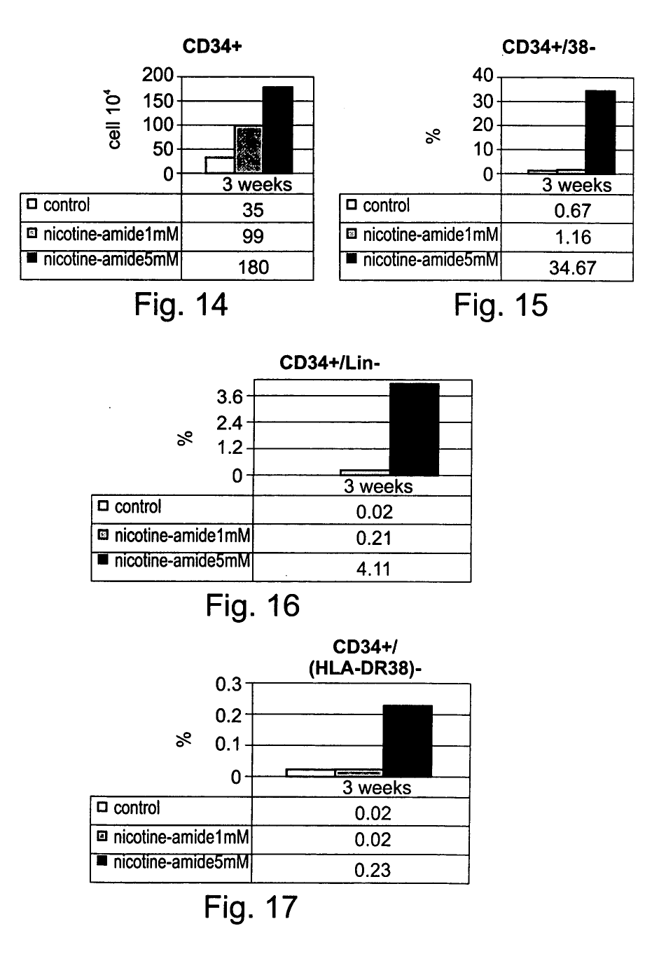

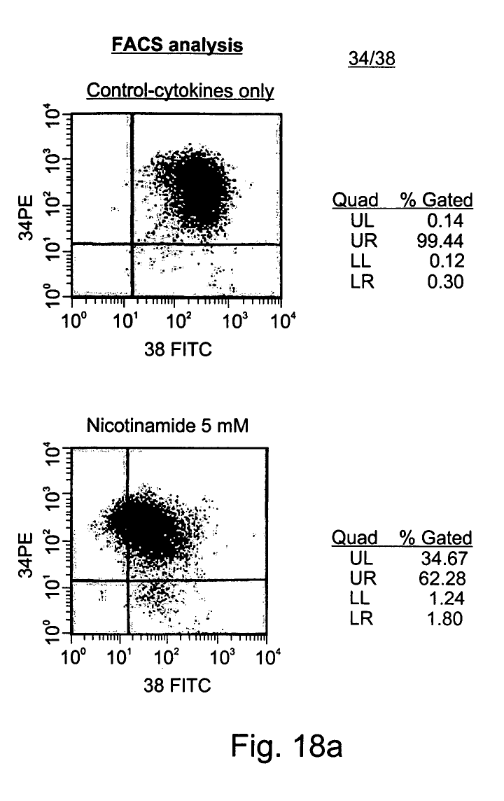

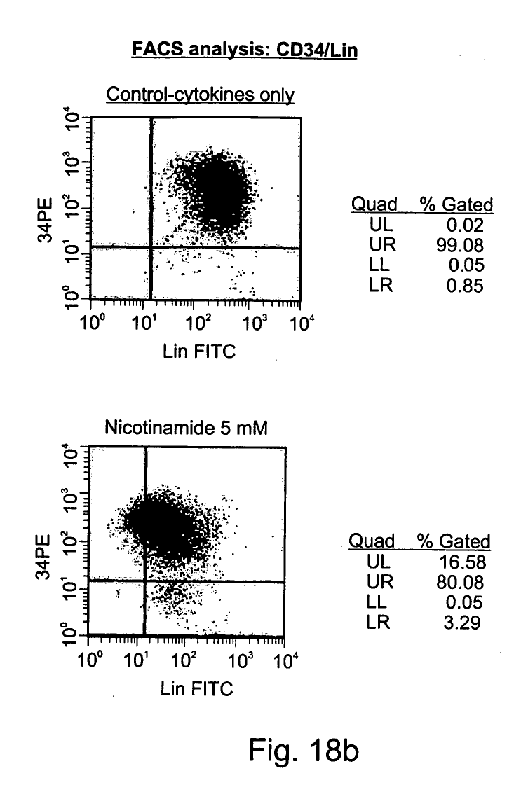

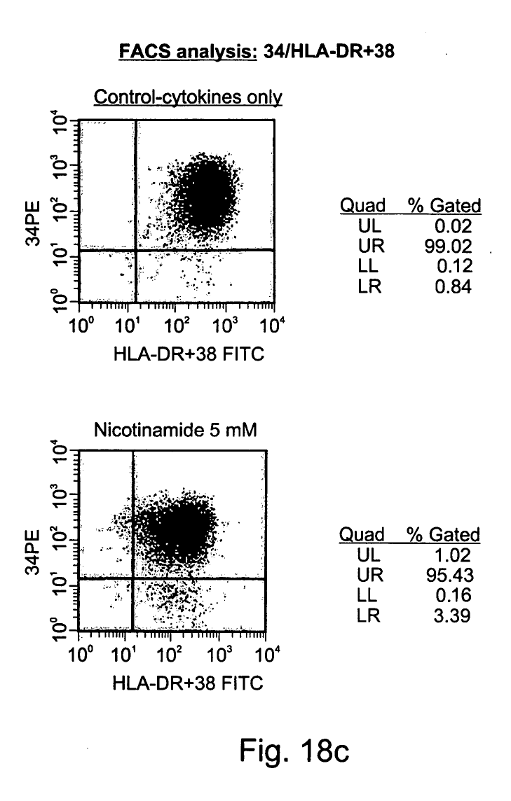

Hematopoietic CD34+ cell cultures were initiated in the presence of a combination of 5 cytokines, SCF, TPO, FLt3, IL-6 and IL-3, with or without different concentrations of nicotinamide. Following three weeks incubation period, the CD34+ cells were re-selected from culture by affinity re-purification method and were enumerated. The results, presented in FIG. 14, show that cultures supplemented with 1 and 5 mM nicotinamide yielded 99×10⁴ and 180×10⁴ CD34+ cells per ml, respectively, as compared with only 35×10⁴ CD34+ cells per ml in the non-treated (cytokines only) control. In addition, the re-selected CD34+ cell fraction was FACS analyzed for stem/progenitor cell markers. The results, presented in FIGS. 15-17 and 18a-b, show substantial increases in the proportion of CD34+/CD38−, CD34+/Lin− and CD34+/(HLA-DR38−) cells in cultures treated with nicotinamide. FIG. 15 shows that cultures supplemented with 1 and 5 mM nicotinamide resulted in 1.7 and 51.7 fold increase, respectively, in CD34+/CD38− cells density, as compared with the untreated (cytokines only) control. FIG. 16 shows that cultures supplemented with 1 and 5 mM nicotinamide resulted in 10.5 and 205.5 fold increase, respectively, in CD34+/Lin− cells density, as compared with the untreated (cytokines only) control. FIG. 17 shows that cultures supplemented with 5 mM nicotinamide resulted in 11.5 fold increase in CD34+/(HLA-DR38−) cells density, as compared with the untreated (cytokines only) control. Hence, nicotinamide was found to be a very effective agent for promoting ex vivo expansion of stem and progenitor cells.

In an additional experiment, cultures were treated with 5 and 10 mM nicotinamide. Table 5 below presents the obtained results, which further demonstrate the powerful effect of nicotinamide on ex-vivo expansion of stem and early progenitor cells.

TABLE 5

| | % of CD34⁺/CD38⁻ of | % of CD34⁺/Lin⁻ of |
|---|---|---|
| Treatment | total cells | total cells |
| control | 1.69 | 0.02 |
| Nicotinamide (5 mM) | 9.69 | 4.11 |
| Nicotinamide (10 mM) | 34.67 | 16.58 |

### Example 6

### Imposing Proliferation yet Restricting Differentiation of Stem and Progenitor Cells by Treating the Cells with Chelators of Transitional Metals

### Experimental Procedures

CD₃₄ cells selection: Peripheral blood “buffy coat” cells derived from a whole blood unit, peripheral blood cells obtained following leukapheresis, or cord blood cells were layered on Ficoll-Hypaque (density 1.077 g/ml) and centrifuged at 1,000×g for 20 min. at room temperature. The interphase layer of mononuclear cells were collected, washed three times with Ca/Mg free phosphate buffered saline containing 1% bovine serum albumin (BSA). The cells were incubated for 30 min. at 4° C. with murine monoclonal anti CD₃₄ antibody (0.5 μg/10⁶ mononuclear cells) and thereafter isolated using the miniMACS apparatus (Miltenyi-Biotec, Bergisch, Gladbach, Germany) according to the manufacturer's protocol.

Culture procedures: For the expansion of progenitor cells, CD₃₄⁺ enriched fractions or unseparated mononuclear cells were seeded at about 1-3×10⁴ cells/ml in either alpha minimal essential medium containing 10% preselected fetal calf serum (FCS) (both from GIBCO, Grand Island, N.Y.), or serum-free medium (Progenitor-34 medium, Life Technologies, Grand Island, N.Y.). The media were supplemented with a mixture of growth factors and transition metal chelators. The cultures were incubated at 37° C. in an atmosphere of 5% CO₂ in air with extra humidity. Half of the medium was changed weekly with fresh medium containing all the supplements.

Cloning potential evaluations: The cloning potential of cells developed in the liquid culture was assayed, at different intervals, in semi-solid medium. The cells were washed and seeded in 35 mm dishes in methylcellulose containing alpha medium supplemented with recombinant growth factors (SCF, G-CSF, GM-CSF and EPO). Following 2 weeks incubation, the cultures were scored with an inverted microscope. Colonies were classified as blast, mixed, erythroid, myeloid, and megakaryocytic, according to their cellular composition.

Morphological assessment: In order to characterize the resulting culture populations, aliquots of cells were deposited on a glass slide (cytocentrifuge, Shandon, Runcorn, UK), fixed and stained in May-Grunwald Giemsa. Other aliquots were stained by benzidine for intracellular hemoglobin.

Immunofluorescence staining: At different intervals, cells from the liquid cultures were assayed for CD₃₄ antigen. Aliquots were harvested, washed and incubated on ice with FITC-labeled anti CD₄₅ monoclonal antibody and either PE-labeled anti CD₃₄ (HPCA-2) monoclonal antibody or PE-labeled control mouse Ig. After incubation, red cells were lysed with lysing solution, while the remaining cells were washed and analyzed by flow cytometer.

Flow cytometry: Cells were analyzed and sorted using FACStar^{plus} flow cytometer (Becton-Dickinson, Immunofluorometry systems, Mountain View, Calif.). Cells were passed at a rate of 1,000 cells/second through a 70 mm nozzle, using saline as the sheath fluid. A 488 mn argon laser beam at 250 mW served as the light source for excitation. Green (FITC-derived) fluorescence was measured using a 530±30 nm band-pass filter and red (PE-derived) fluorescence—using a 575±26 nm band filter. The PMTs was set at the appropriate voltage. Logarithmic amplification was applied for measurements of fluorescence and linear amplification—for forward light scatter. At least 10⁴ cells were analyzed.

### Experimental Results

In an effort to develop culture conditions which stimulate proliferation and inhibit differentiation of hematopoietic progenitor cells, CD₃₄⁺ cells were cultured with the following supplements:

Transition metal chelators such as—tetraethylpentamine (TEPA), captopril (CAP) penicilamine (PEN) or other chelators or ions such as Zinc which interfere with transition metal metabolism;

Early-acting cytokines—stem cell factor (SCF), FLT3 ligand (FL), interleukin-6 (IL-6), thrombopoietin (TPO) and interleukin-3 (IL-3);

Late-acting cytokines—granulocyte colony stimulating factor (G-CSF), granulocyte/macrophage colony stimulating factor (GM-CSF) and erythropoietin (EPO).

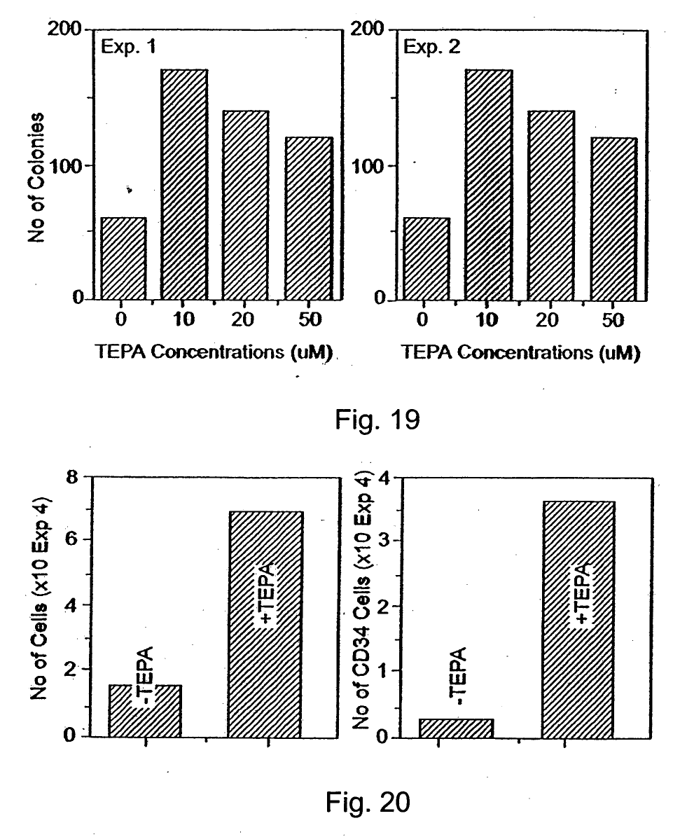

TEPA effects on proliferation and clonability of short term CD₃₄+ cultures: Addition of TEPA to CD34⁺ cells cultured with low doses of early-acting cytokines resulted in a significant increase in total cell number, in the number of CD₃₄⁺ cells (measured by flow cytometry utilizing fluorescence labeled specific antibodies, FIG. 20) and in cell clonability (measured by plating culture aliquots in semi-solid medium and scoring colonies that develop two weeks later, FIG. 19), compared to cultures supplemented only with cytokines. The colonies which developed in semi-solid medium in the presence of TEPA were of myeloid, erythroid and mixed phenotype.

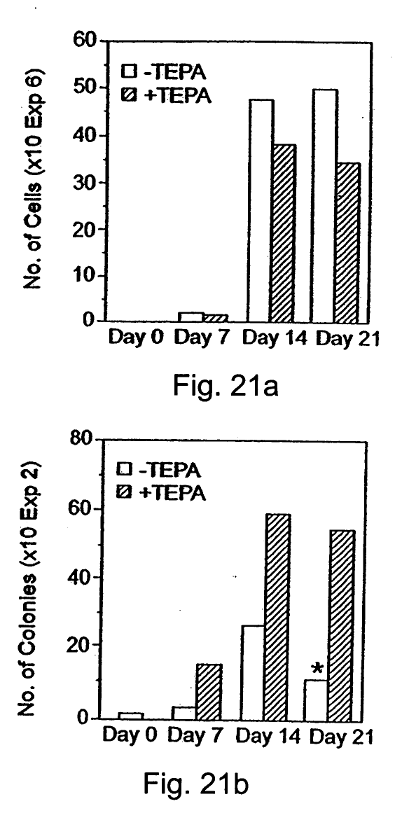

The effects of TEPA were further assessed in cultures supplemented with either high doses of early cytokines (Table 6) or with a combination of early- and late-acting cytokines (FIGS. 21a-b). The results indicated that TEPA significantly increased the clonability and the percentage of CD₃₄⁺ cells in these cultures. As for total cell number it was increased by TEPA in cultures supplemented with early cytokines (Table 6; FIG. 20), whereas in cultures supplemented with both early and late cytokines, TEPA caused a marginal inhibition (FIGS. 21a-b).

Cord blood-derived CD₃₄ cells were plated in liquid culture in the presence of: FL—50 ng/ml, SCF—50 ng/ml, IL-6—50 ng/ml, with or without IL-3—20 ng/ml, with or without TEPA—10 μM. On day 7, the percentage of CD₃₄ cells and the total cell number were determined. Aliquots equivalent to 1×10³ initiating cells were assayed on days 0 and 7 for colony forming cells (CFU) by cloning in semi-solid medium. CFU expansion represents the ratio of CFU present on day 7 to CFU present on day 0.

TABLE 6
The short-term effect of TEPA on CD₃₄ cells

| | | | | Colonies | CFU |
|---|---|---|---|---|---|
| | | Cells/ml | CD₃₄ cells | (Per 1 × 10³ | expansion |
| TEPA | Il-3 | (×10⁴) | (%) | initiating cells) | (fold) |
| − | − | 1 | 1 | 16 | 0.3 |
| + | − | 2 | 11.5 | 140 | 2.8 |
| − | + | 5 | 5 | 165 | 3.3 |
| + | + | 11 | 20 | 850 | 17 |

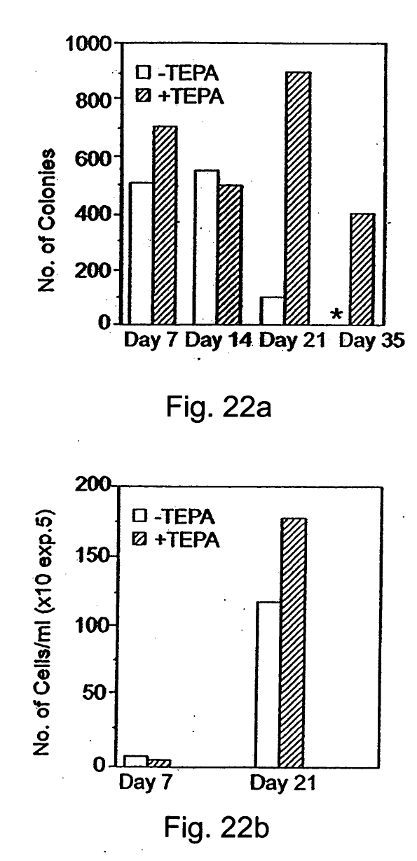

TEPA effects on proliferation and clonability of long-term CD34⁺ cultures: Long-term cultures were maintained for 3-5 weeks by weekly demi-depopulation (one half of the culture volume was removed and replaced by fresh medium and cytokines). Addition of TEPA resulted in a higher clonability in long-term cultures supplemented with either early cytokines (FIGS. 22a-b) or both early and late cytokines (FIGS. 21a-b), as compared to cultures supplemented only with cytokines.

After three weeks in culture, there was a sharp decrease in clonability in cultures supplemented only with cytokines, whereas cultures treated with TEPA in combination with cytokines maintained high clonability, which was even higher than that of short-term cultures.

The effect of TEPA on the maturation of hematopoietic cells: The effect of TEPA on the maturation of hematopoietic cells was tested on several models:

Mouse erythroleukemic cells (MEL): MEL cells are erythroblast like cells. Following treatment with several chemicals (differentiation inducers) the cells undergo erythroid differentiation and accumulate hemoglobin. MEL cells were cultured in the presence of the differentiation inducer hexamethylene bisacetamide (HMBA) and the chelators TEPA or Captopril. At day 3 of the culture, the total number of cells and the percentage of hemoglobin-containing cells were determined (Table 7). The results indicated that both TEPA and captopril inhibited the HMBA-induced differentiation of MEL cells.

Human erythroid cell cultures: Normal human erythroid cells were grown according to the two-phase liquid culture procedure, essentially as described in references 67-70. In the first phase, peripheral blood mononuclear cells were incubated in the presence of early growth factors for 5-7 days. In the second phase, these factors were replaced by the erythroid specific proliferation/differentiation factor, erythropoietin.

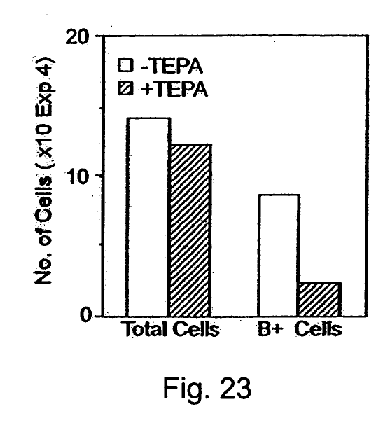

The cultures were supplemented with TEPA at the initiation of the second phase. The total cell number and the percentage of hemoglobin-containing cells were determined after 14 days. The results (FIG. 23) showed that in the presence of TEPA there was a sharp decrease in hemoglobin-containing cells, while the total number of cells decreased only slightly.

These results suggest that TEPA inhibits erythroid differentiation, but does not significantly affect the proliferation ability of the progenitor cells.

TABLE 7
The effect of TEPA and captopril on
growth and differentiation of erythroleukemic cells

| | | Benzidine Positive |
|---|---|---|
| | Cells/ml (×10⁴) | Cells (%) |
| Control | 31 | <1 |
| HMBA | 32 | 46 |
| HMBA + TEPA 5 μM | 35 | 24 |
| HMBA + TEPA 10 μM | 35 | 16 |
| HMBA + TEPA 20 μM | 47 | 16 |
| HMBA + Captopril 20 μM | 34 | 29 |
| HMBA + Captopril 40 μM | 34 | 12 |

Murine erythroleukemia cells (MEL), were cultured in liquid medium supplemented with the differentiation inducer—hexamethylene-bisacetamide (HMBA, 4 mM), with or without different concentrations of TEPA or captopril. On day 3, total cell number and hemoglobin containing (benzidine positive) cells were determined.

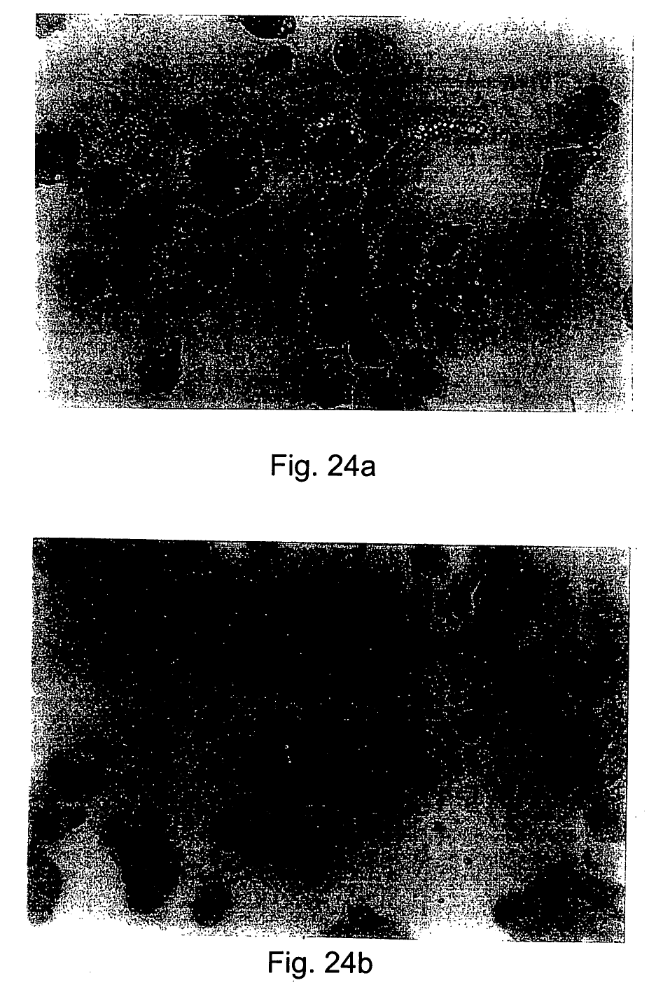

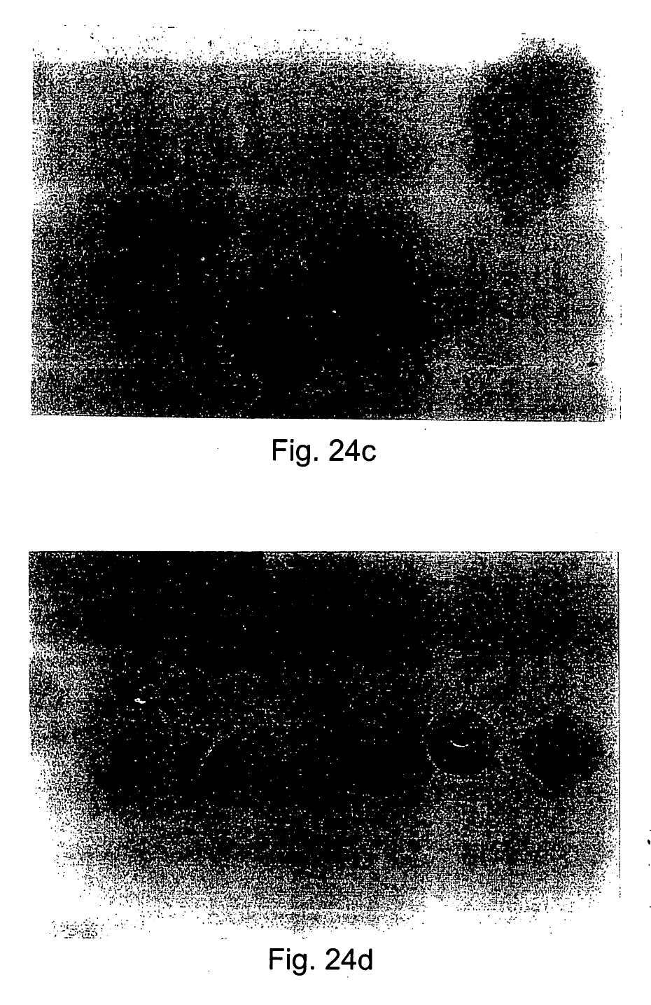

CD₃₄⁺ initiated cultures: Long term liquid cultures initiated with CD₃₄⁺ cells were maintain with different cocktails of cytokines. Half of the cultures were continuously supplemented with TEPA. In order to test the status of cell differentiation, cytospin preparation were stained with May-Grunwald Giemsa (FIGS. 24a-d). The results showed that cultures which were maintained for 4-5 weeks without TEPA contained only fully differentiated cells, while with TEPA the cultures contained, in addition to fully differentiated cells, a subset of 10%-40% of undifferentiated blast-like cells.

These results strongly suggest that TEPA induces a delay in CD₃₄⁺ cell differentiation which results in prolonged proliferation and accumulation of early progenitor cells in long-term ex-vivo cultures.

TEPA s mechanism of activity: In order to determine whether TEPA affects CD₃₄⁺ cells via depletion of transition metals, such as Copper, two approaches were taken.

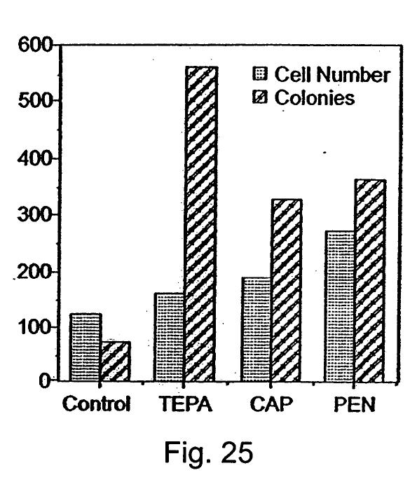

The first was to assess the effect of different transition metal chelators: tetra-ethylpentamine (TEPA), captopril (CAP) or penicilamine (PEN). The results demonstrated that all these compounds share the same effects on CD₃₄⁺ cells as TEPA (FIG. 25).

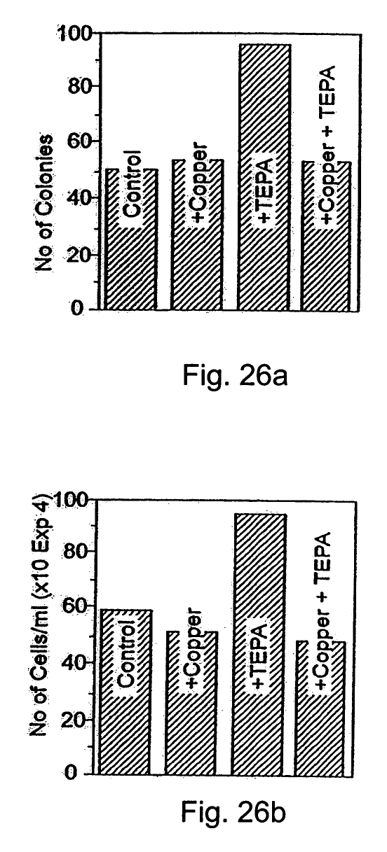

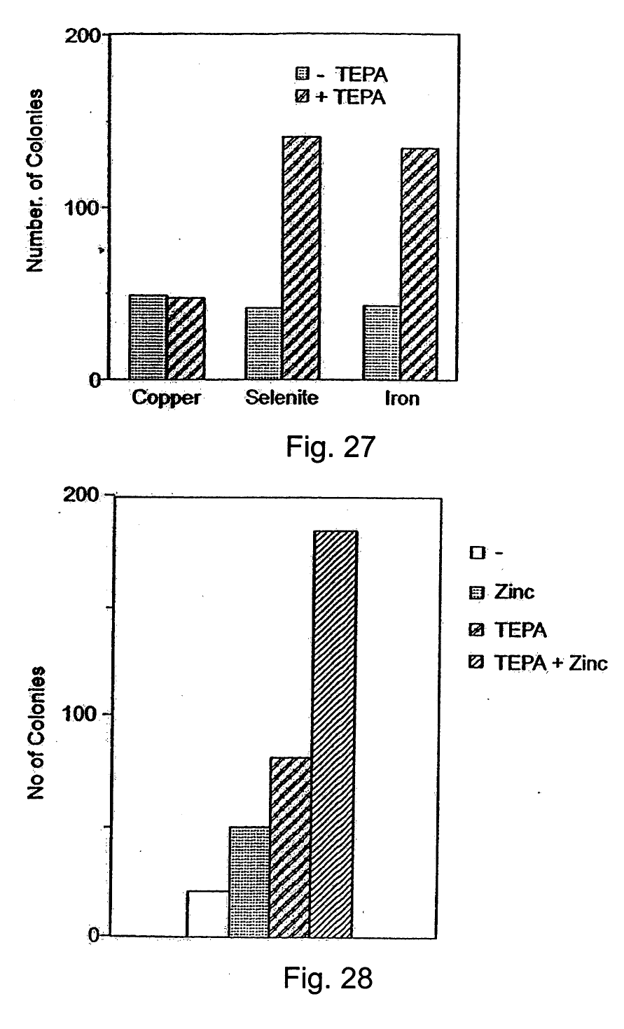

The second approach was to supplement TEPA-treated cultures with Copper. The results indicated that TEPA activities were reversed by Copper (FIGS. 26a-b), while supplementation with other ions, such as iron and selenium, did not (FIG. 27), at least in the short to medium term cultures employed herein.

Zinc, which is known to interfere with transition metal metabolism, e.g., with Copper metabolism, expand the clonability of the cultures by itself. This effect was even more pronounced in the presence of both Zinc and TEPA (FIG. 28).

In the above examples it is demonstrated that by supplementing CD₃₄ cell cultures with early-acting cytokines and the polyamine agent—tetraethylenepentamine (TEPA), for example, it is possible to maintain long term cultures (LTC) without the support of stroma. Three phenomena were evident in these cultures: (i) continuos cell proliferation; (ii) expansion of clonogenic cells (CFUc); and (iii) maintenance of cells at their undifferentiated status.

In contrast, control, TEPA-untreated cultures ceased to proliferate and to generate CFUc and their cells underwent differentiation much earlier.

Thus, TEPA and other transition metal chelators sustains long-term cultures by inhibiting/delaying cellular differentiation through chelation of transition metals, Copper in particular.

The following example further substantiate the results described hereinabove; teaches optimal culture conditions for long-term cultures, teaches additional chelating agents that affect hematopoietic cell differentiation and sheds more light on the mechanism of activity of TEPA and other chelators on their target cells.

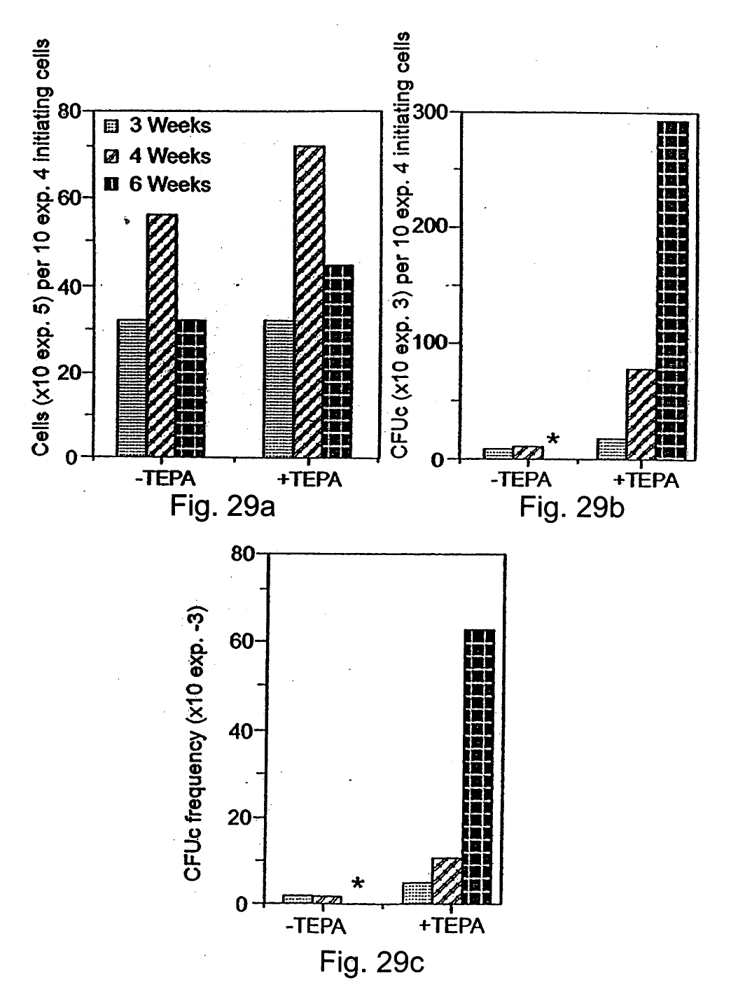

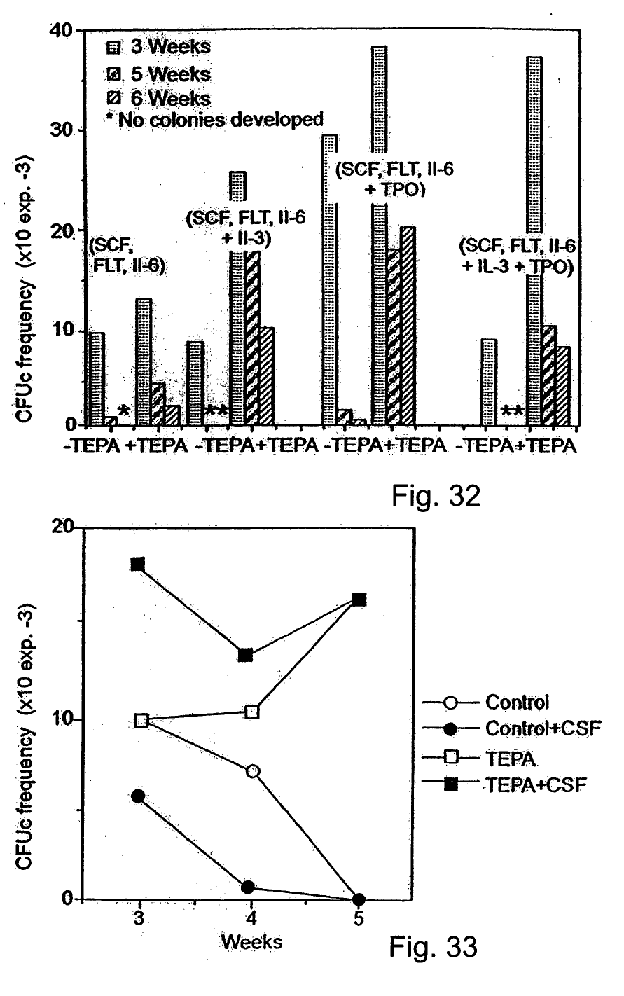

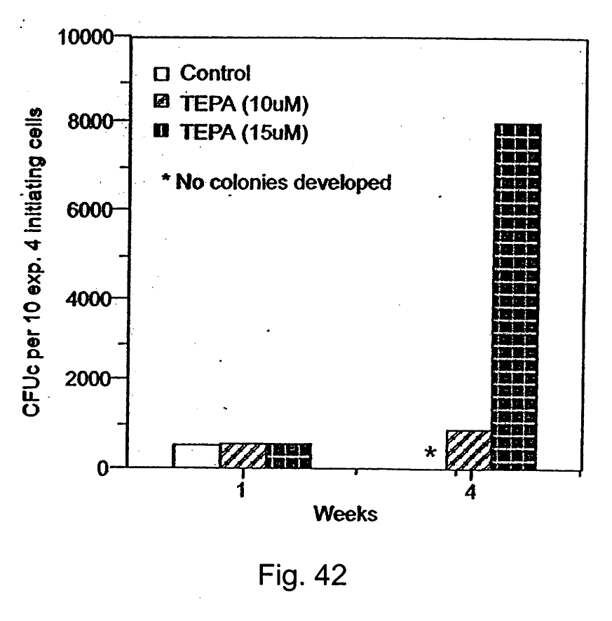

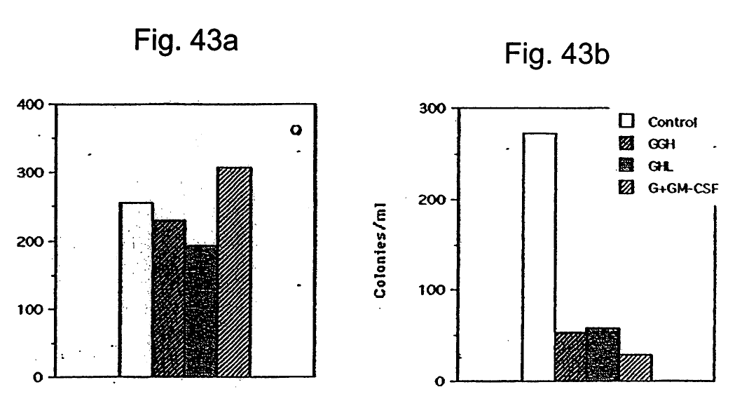

CD₃₄⁺ cells derived from human neonatal cord blood were purified by immunomagnetic method and then cultured in liquid medium supplemented with cytokines either with or without transition metal chelators. At weekly intervals, the cultures were demi-depopulated by removing half of the culture content (supernatant and cells) and replacing it with fresh medium, cytokines and the chelators. At the indicated weeks the cellular content of the cultures were quantified for total cells (by a manual microscopic/hemocytometric method), for CD₃₄⁺ cells (by immuno-flow cytometry) and for clonogenic cells (by cloning the cells in cytokine-supplemented semi-solid medium). The cultures were initiated with 1×10⁴ cells, 50-80% of which were CD₃₄⁺ and 25-50% of which were CFUc. The results presented in FIGS. 29 to 42 were calculated per 1×10⁴ initiating cells (the numbers were multiplied by the dilution factors).

FIG. 29 shows the effect of TEPA on long-term CD₃₄ cultures. Cultures initiated with CD₃₄ cells in liquid medium supplemented with early-acting cytokines (in the absence of stromal cells) could be maintained by TEPA for a long time (>6 weeks). In such cultures, TEPA supported, in combination with the cytokines, maintenance and expansion of clonogenic cells (CFUc): The cultures were started with 2.5×10³ CFUc. Upon termination after 6 weeks, TEPA-treated cultures contained 300×10³ CFUc, (i.e., a 120-fold expansion) while control cultures contained no CFUc.

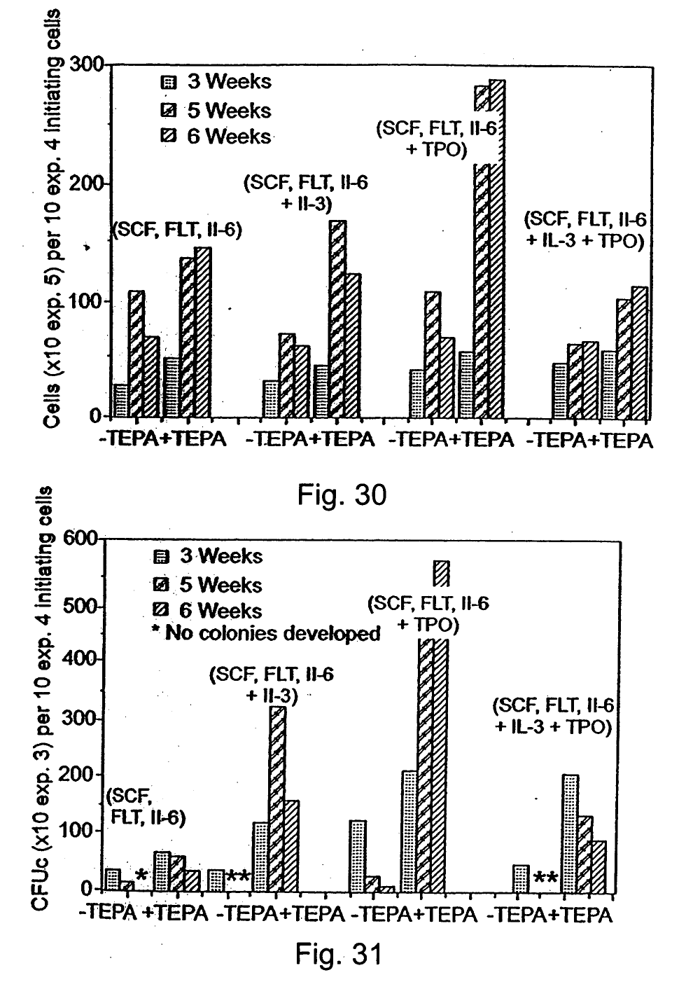

FIGS. 30-32 show the effect of TEPA on cell proliferation, CFUc and CFUc frequency in the presence of different combination of early cytokines. The combination of the early-acting cytokines TPO, SCF, FLT, IL-6 and TEPA was found to be the optimal combination for the maintenance and long term expansion of cells with clonogenic potential.

FIG. 33 shows the effect of G-CSF and GM-CSF on CFUc frequency of control and TEPA-supplemented CD₃₄ cultures. Supplementing the cultures with the late-acting cytokines G-CSF and GM-CSF, which stimulate cell differentiation, resulted in rapid loss of clonogenic cells. This differentiation stimulatory effect is blocked by TEPA.

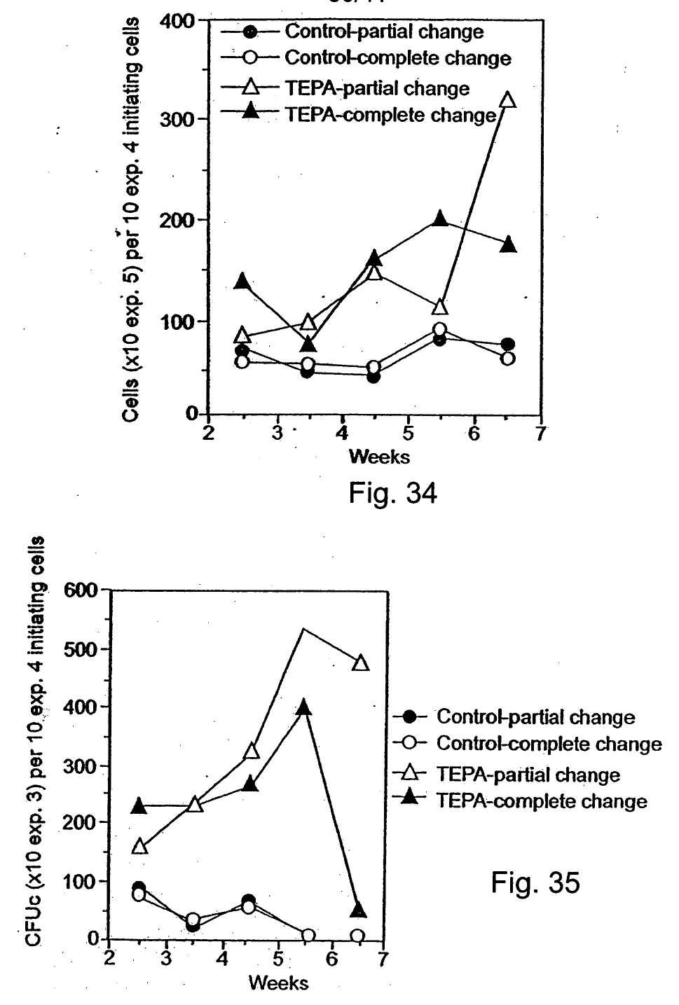

FIGS. 34-35 show the effect of partial or complete medium+TEPA change on long-term cell proliferation (FIG. 34) and CFUc production (FIG. 35). The results obtained indicate that for maintaining maximal expansion, TEPA should be completely replaced, at least, at weekly intervals.

FIG. 37 shows the effect of delayed addition of TEPA on CFUc frequency. It is evident that early exposure of CD₃₄ cells to TEPA was crucial for long-term maintenance and expansion of CFUc, suggesting that TEPA affects differentiation of progenitors at various stages of differentiation.

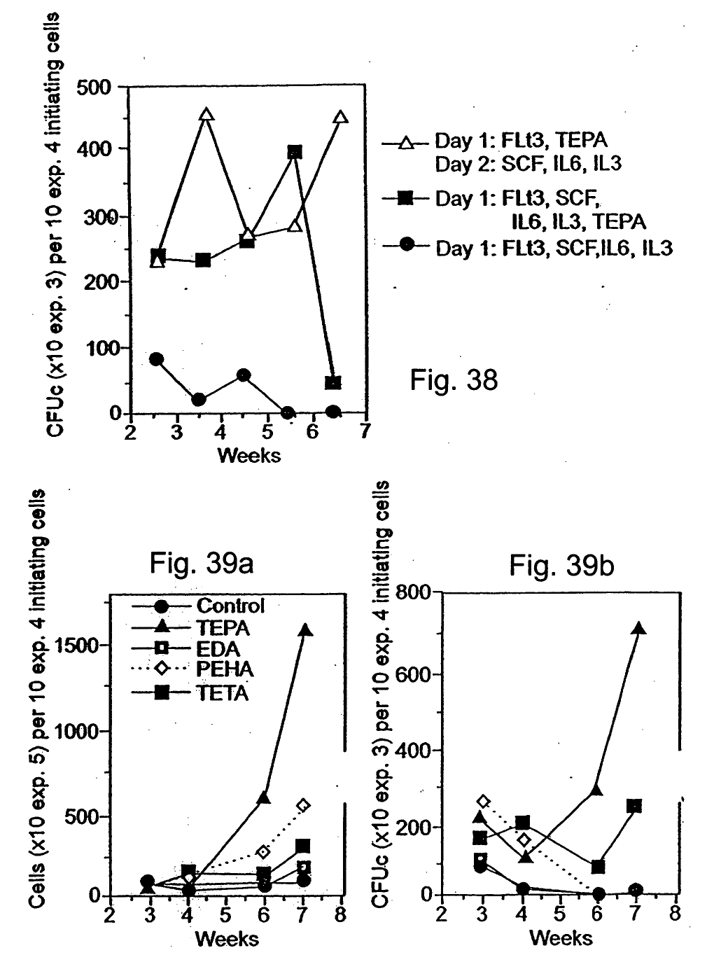

FIG. 38 shows the effect of short-term preincubation with a single cytokine on long-term CFUc production. The results indicate that LTC-CFC are more preserved in TEPA-treated cultures when supplemented for the first 24 hours with a single cytokine rather than the full complement of cytokines, suggesting that under the former conditions cells are blocked more efficiently.

FIGS. 39a-b show the effect of polyamine chelating agents on CD₃₄ cell cultures. Polyamine chelating agents sustained cell proliferation and expanded CFUc during long term cultures. Among the compounds tested, the long-chain polyamines, TEPA and PEHA, were found to be more effective than the short-chain polyamines.

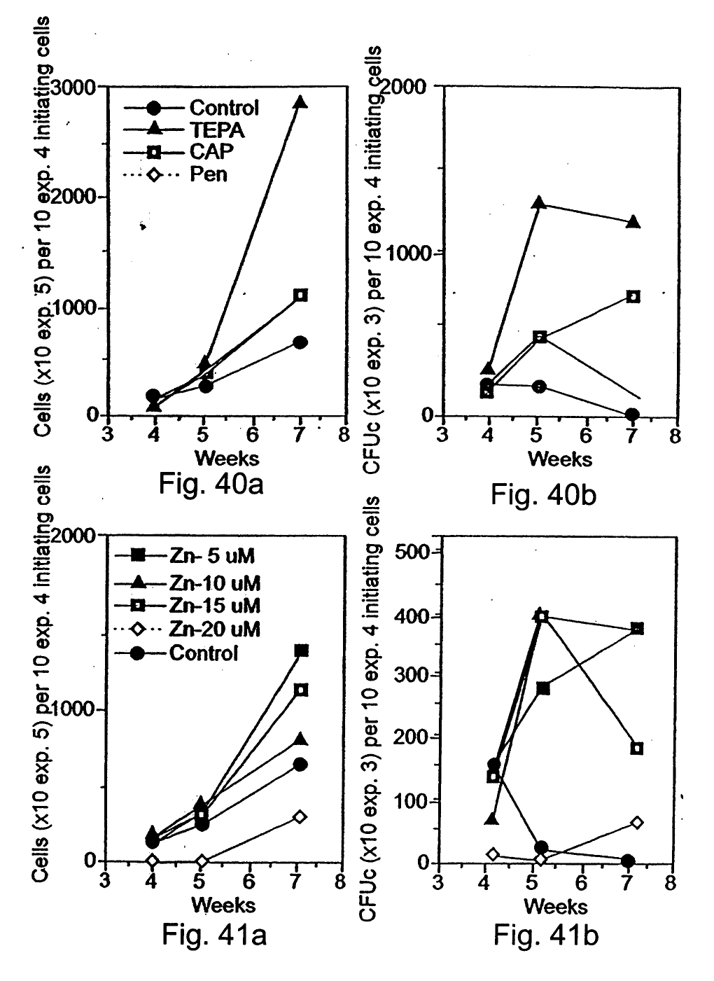

FIGS. 40a-b show the effect of transition metal chelating agents on CD₃₄ cell cultures. Penicilamine (PEN) and captopril (CAP), which are known transition metal chelators, sustained cell proliferation and expansion of clonogenic cells during long-term cultures.

FIGS. 41a-b show the effect of Zinc on CD₃₄ cell cultures. Zinc, which is known to interfere with transition metal metabolism, Copper in particular, mimicked the effect of the chelating agents in long term cultures, but to a smaller extent than the chelators themselves.

Thus, ex-vivo expansion of hematopoietic progenitor cells is limited by the progression of these cells into non-dividing differentiated cells. This differentiation process can be delayed by cultivating the progenitor cells on stroma cell layer. Since the stroma supports continuous cell proliferation and long-term generation of CFUc, it is believed that the stroma inflict an anti differentiation effect on the progenitor cells.

According to another embodiment of the present invention there is provided a method of preservation of stem cells, such as, but not limited to, cord blood derived stem cells, peripheral blood derived stem cells and bone marrow-derived stem cells. The method is effected by handling the stem cell while being harvested, isolated and/or stored, in a presence of a transition metal chelator, e.g., TEPA.

Cord blood-derived cells were collected and stored (unseparated) for 24 hours, at 4° C., either in the presence or absence of 10 μM TEPA. CD₃₄⁺ cells were then separated using either 10 μM TEPA-PBS buffer or TEPA free PBS buffer, respectively. Then, cells were grown in long-term cultures in the presence of 10 μM TEPA.

The results indicated that cultures which were initiated with cells that were handled in the presence of TEPA expanded for 8 weeks, whereas cultures initiated from cells stored without TEPA stopped expanding after 5 weeks only.

It is well known that it takes usually at least several hours between cell collection and either freezing or transplantation.

These results indicate that addition of a transition metal chelator, such as TEPA, to the collection bags and the separation and washing buffers increase the yield of stem cells and improve their potential for long-term growth, thus facilitate the short-term take and the long-term repopulation following transplantation of either “fresh”, cryopreserved or ex-vivo expanded hematopoietic cells.

Thus, further according to the present invention there are provided stem cells collection bags and separation and washing buffers supplemented with an effective amount or concentration of transition metal chelator, which inhibits differentiation.

As is specifically demonstrated in the above examples, a novel system which sustains continuous cell proliferation and long-term generation of CFUc in stroma-free cultures (FIG. 29) has been developed. The system combines the use of early-acting cytokines, such as stem cell factor (SCF), FLT3, interleukin-6 (IL-6), thrombopoietin (TPO) with or without interleukin-3, and transition metal chelating agents (FIGS. 30-32). The early cytokines support the survival and proliferation of the progenitors with reduced stimulus for differentiation compared to late-acting cytokines, such as G-CSF and GM-CSF (FIG. 33). The chelators inhibit differentiation through chelation of transition metals, Copper in particular. Complete medium change at weekly intervals, as compared to partial change, improved LTC-CFC maintenance, suggesting that the TEPA-transition metal complex, e.g., TEPA-Copper complex, may not be stable (FIGS. 34-35).

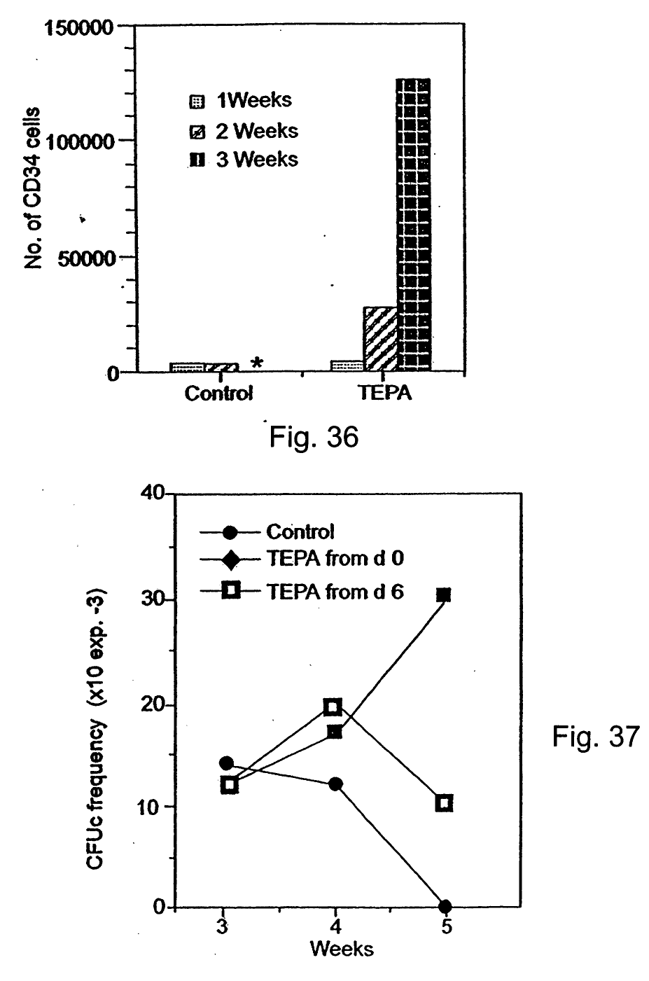

Several lines of evidence suggest that TEPA inhibits differentiation of early progenitors (FIG. 36). For example, when TEPA addition was delayed until day 6 of the culture its effects were reduced as compared to cultures supplemented with TEPA from day 1 (FIG. 37).

While optimal results were obtained when TEPA was added on day 1, it was advantageous to add the full complement of cytokines on day 2. Thus, TEPA-treated cultures which were supplemented for one day with only one cytokine, e.g., FLT3, followed by addition of the other cytokines (SCF, TPO and IL-3) were maintained longer than cultures where all the cytokines were added at day 1 (FIG. 38). We hypothesize that since cell differentiation is driven by the cytokines and is dependent on Copper and other transition metals, inhibition of differentiation requires depletion thereof prior to exposure to the full complement of cytokines. A single cytokine does not support rapid activation (proliferation and differentiation) but maintains cell viability, thus allowing TEPA to efficiently chelate transition metals in quiescent undifferentiated CD₃₄ cells prior to activation.

Following screening, various chelating agents have been found to support continuous cell proliferation and long-term generation of CFUc and to delay cell differentiation. Among them are the polyamines such as, but not limited to, TEPA, EDA, PEHA and TETA (FIGS. 39a-b) or chelators such as, but not limited to, penicilamine (PEN) and captopril (CAP) (FIGS. 40a-b). Zinc which interfere with transition metals (Copper in particular) metabolism also supported LTC-CFC (FIGS. 41a-b).

### Example 7

### The Effect of Copper-chelating Peptides on Proliferation and Clonability in CD₃₄ Cell Cultures

### Experimental Procedures

CD₃₄ cells selection: Peripheral blood “buffy coat” cells derived from a whole blood unit, peripheral blood cells obtained following leukapheresis, or blood cells were layered on Ficoll-Hypaque (density 1.077 g/ml) and centrifuged at 1,000×g for 20 minutes at room temperature. The interphase layer of mononuclear cells were collected, washed three times with Ca/Mg free phosphate buffered saline containing 1% bovine serum albumin (BSA). The cells were incubated for 30 minutes at 4° C. with murine monoclonal anti CD₃₄ antibody (0.5 μg/10⁶ monoclonal cells) and thereafter isolated using the miniMACA apparatus (Miltenyi-Biotec, Bergisch, Gladbach, Germany) according to the manufacturer's protocol.

Culture procedures: For the expansion of progenitor cells, CD₃₄⁺ enriched fractions were seeded at 1×10⁴ cells/ml in alpha minimal essential medium containing 10% preselected fetal calf serum (FCS) (both from GIBCO, Grand Island, N.Y.). The medium was supplemented with a mixture of growth factors and Copper chelators. The cultures were incubated at 37° C. in an atmosphere of 5% CO₂ in air with extra humidity. Half of the medium was changed weekly with fresh medium containing all the supplements.

The cloning potential of the cultured cells was assayed in semi-solid medium. The cells were washed and seeded in 35 mm dishes in methylcellulose containing alpha medium supplemented with 30% FCS and further with recombinant growth factors (stem cell factor (SCF), G-CSF, GM-CSF and erythropoietin (EPO)). Following two week incubation, the cultures were scored with an inverted microscope. Colonies were classified as blast, mixed, erythroid, myeloid, and megakaryocytic, according to their cellular composition.

Morphological assessment: In order to characterize the resulting culture populations, aliquots of cells were deposited on a glass slide (cytocentrifuge, Shandon, Runcorn, UK), fixed and stained in May-Grunwald Giemsa.

Immunofluorescence stainingfor CD₃₄ antigen: Cells were incubated on ice with FITC-labeled anti CD₄₅ monoclonal antibody and either phycoerythrin (PE)-labeled anti CD₃₄ (HPCA-2) monoclonal antibody or PE-labeled control mouse Immunoglobulins (Ig). After incubation, the cells were washed and analyzed by flow cytometry.

Flow cytometry: Cells were analyzed using FACStar^{plus} flow cytometer (Becton-Dickinson, Immunofluorometry systems, Mountain View, Calif.). Cells were passed at a rate of 1,000 cells/second through a 70 μm nozzle, using saline as the sheath fluid. A 488 nm argon laser beam at 250 mW served as the light source for excitation. Green (FITC-derived) fluorescence was measured using a 530±30 nm band-pass filter and red (PE-derived) fluorescence—using a 575±26 nm band filter. The PMTs was set at the appropriate voltage. Logarithmic amplification was applied for measurements of fluorescence and linear amplification—for forward light scatter. At least 10⁴ cells were analyzed.

### Experimental Results

The effect of Copper-chelating peptides on proliferation and clonability in CD₃₄ cell cultures: Cultures were initiated with 10⁴ cord blood-derived CD₃₄⁺ cells by plating purified cells in liquid medium in the presence of SCF, FLT3 and IL-6 (50 ng/ml each) and the Copper-binding peptides, Gly-Gly-His (GGH) or Gly-His-Lys (GHL) (10 μM each), or the late-acting cytokines G-CSF and GM-CSF (10 ng/ml each). At weekly intervals, the cultures were demi-depopulated and supplemented with fresh medium, cytokines and the peptides. After 7 weeks, cells were counted and assayed for CFUc.

As shown in FIGS. 53a-b, the results indicated that GGH and GHL decreased cell number by 10% and 25%, respectively, and G-CSF+GM-CSF by 20%. The effect on the clonogenic potential of the cultures was much more pronounced: 80% and 78% decrease by GGH and GHL, respectively, and 89% by G-CSF+GM-CSF.

### Example 8

### Transition Metal Chelator Assay for Determining the Effect of a Specific Transition Metals Chelator on Cell Differentiation

### Experimental Procedures

Inhibition of differentiation: MEL (mouse erythroleukemia cell line), 8×10³ cells per ml were incubated for 24 hours with different chelators at concentrations indicated in Table 8 below. Then, cultures were supplemented with a differentiation inducer—hexamethylene bisacetamide, 2 mM. Number of cells and percentage of differentiated cells (benzidine positive) were determined 72 hours after addition of the inducer.

Similarly, HL-60 (human myeloid leukemia cell line), 1×10⁵ cells per ml were incubated for 24 hours with different chelators at the concentrations indicated in Table 8 below. Then, cultures were supplemented with the differentiation inducers—vitamin D or retinoic acid (both at 1×10⁻⁷ M). Number of cells and percentage of differentiated (phagocytosing) cells were determined.

Induction of differentiation: HL-60, 1×10⁵ cells per ml were incubated with different chelators. Number of cells and percentage of differentiated (phagocytosing) cells were determined.

Copper Determination: Cells were harvested by centrifugation at 1000×g for 5 minutes. The cell pellet was washed three times by re-suspending the cells in PBS (Ca⁺⁺ and Mg⁺⁺ free) and centrifugation at 1000×g. An aliquot containing 2×10⁶ cells was then transferred into a metal-free Eppendorf tube and the cells were recovered by centrifugation at 1000×g. The cell pellet was re-suspended in 0.03 M ultra-pure nitric acid to give a concentration of 1×10⁷ cells/ml. The cells were homogenized with a high shear mixer (polytron, Kinematica, Switzerland) for 1 minutes to disrupt the cell and release intracellular copper content. Cell samples were vortexed before transferring to a vial autosampler and analyzed in duplicate by a Perkin Elmer graphite furnace atomic absorption spectrophotometer at a wavelength of 324.7 nm. The samples were analyzed against copper standard solution prepared from a commercial stock solution that was diluted with 0.03 M ultra pure nitric acid.

### Experimental Results

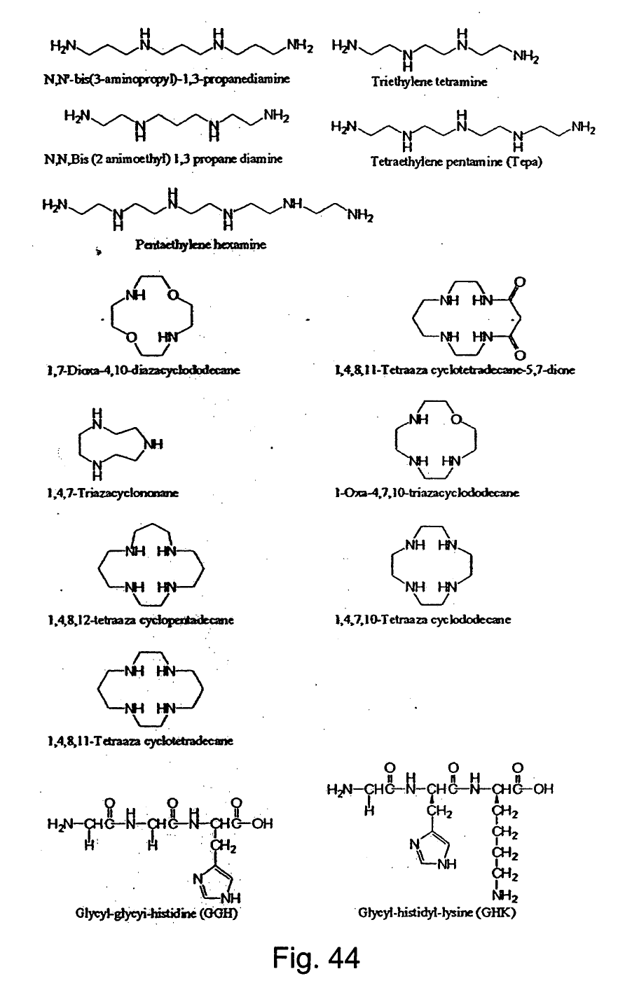

Table 8 bellow summarized the results for HL-60 cells. Inhibition of differentiation of MEL cells yielded comparable results. FIG. 44 provides the chemical structure of the various chelators employed in these experiments.

TABLE 8
Positive correlation between the ability of copper chelators to inhibit or
induce differentiation and copper content in chelator treated cells

| Compound | | | | | | | | | | | | | |
|---|---|---|---|---|---|---|---|---|---|---|---|---|---|
| | Copper | Differentiation | | | | growth | | | | | | | |
| | Affinity | Inhibition | | Induction | | inhibition | | Average Intracellular ppb Cu | | | | | |
| Name | (LogK | 100 | 1000 | 100 | 1000 | 100 | 1000 | (% of control) | | | | | |
| Concentration tested | Cu) | nM | nM | nM | nM | nM | nM | 20 μM | | 100 μM | | 500 μM | |
| Control | | | | | | | | 49 + − 18 | | | | | |
| N,N′-bis(3-amino | 17.3 | − | − | − | − | − | − | 33.8 ppb | 69% | 26.2 ppb | 53% | 27.9 ppb | 57% |
| propyl)-1,3-propanediamine | | | | | | | | | | | | | |
| Triethylene tetramine | 20.2 | + | + | − | − | − | + | 27.7 ppb | 56% | 21.2 ppb | 43% | 16.8 ppb | 34% |
| N,N,Bis (2 animoethyl) 1,3 | 23.9 | + | + | − | − | + | + | 10.8 ppb | 22% | 13.4 ppb | 27% | ND | |
| propane diamine | | | | | | | | | | | | | |
| Tetraethylene pentamine | 24.3 | + | + | − | − | − | + | 31.5 ppb | 64% | 24.1 ppb | 49% | 17.1 ppb | 35% |
| (TEPA) | | | | | | | | | | | | | |
| Pentaethylene hexamine | | + | + | − | − | − | + | 19.3 ppb | 39% | 24.5 ppb | 50% | 17.2 ppb | 35% |
| 1,7-Dioxa-4,10- | | − | − | − | − | − | − | 35.5 ppb | 72% | 36.1 ppb | 73% | 35.0 ppb | 71% |
| diazacyclododecane | | | | | | | | | | | | | |
| 1,4,8,11-Tetraaza | 15 | − | − | − | − | − | − | 37.9 ppb | 77% | 27.4 ppb | 56% | 28.3 ppb | 57% |
| cyclotetradecane-5,7-dione | | | | | | | | | | | | | |
| 1,4,7-Triazacyclononane | 15.5 | + | + | − | − | − | − | 15.8 ppb | 32% | 17.7 ppb | 36% | ND | |
| trihydrochloride | | | | | | | | | | | | | |
| 1-Oxa-4,7,10- | | + | + | − | − | − | − | 39.0 ppb | 79% | 22.9 ppb | 46% | 17.6 ppb | 36% |
| triazacyclododecane | | | | | | | | | | | | | |
| 1,4,8,12-tetraaza | 24.4 | + | + | − | − | + | + | 13.4 ppb | 27% | 12.1 ppb | 25% | 9.6 ppb | 19% |
| cyclopentadecane | | | | | | | | | | | | | |
| 1,4,7,10-Tetraaza | 24.8 | − | − | + | Toxic | | | 27.5 ppb | 56% | 73.9 ppb | 150% | Toxic | |
| cyclododecane | | | | | | | | | | | | | |
| 1,4,8,11-Tetraaza | 27.2 | + | + | − | − | + | + | 15.0 ppb | 30% | 11.4 ppb | 23% | 19.9 ppb | 40% |
| cyclotetradecane | | | | | | | | | | | | | |
| Glycyl-glycyl- | | − | − | + | + | + | + | 202.7 ppb | 411% | 582 ppb | 1181% | 1278 ppb | 2592% |
| histidine Cu complex (GGH- | | | | | | | | | | | | | |
| Cu) | | | | | | | | | | | | | |
| Glycyl-histidyl-lysine Cu | | − | − | + | + | + | + | 481 ppb | 976% | 473 ppb | 959% | 1066 ppb | 2162% |
| complex (GHK-Cu) | | | | | | | | | | | | | |

ND—not determined;
ppb—parts per billion.

As is evident from Table 8 above, good correlation was found between the ability of chelators to modulate cellular copper content and their biological activities. Chelators that reduce cellular copper content are potent differentiation inhibitors. On the other hand, chelators that increase cellular copper content are potent differentiation inducers. Indeed, differentiation inhibitory chelators, such as TEPA, PEHA etc., when tested for their activity on CD₃₄₊ cells, were found to inhibit differentiation. Chelators with differentiation inducing activity such as 1,4,7,10-Tetra-azacyclododecane and the copper binding peptides GGH and HHK were found to stimulate differentiation. Therefore, screening for the ability of chelators to modulate (increase or decrease) cellular copper content could be a predictive assay for the effect of the chelators on various cell types such as the hematopoietic stem (CD₃₄₊) cells.

### Example 9

### Modulation of Differentiation by Copper Chelators on Non-hematopoietic Cells

As is indicated in the Background section above, and as is known from the scientific literature, cooper depletion in-vivo affects a plurality of cell lineages, including, but hematopoietic cells. It was therefore anticipated that the effect of transition metal chelators on differentiation is not limited to cells of the hematpoietic lineage, rather this effect is an underlying phenomenon shared by all eukaryotic cells.

Embryonal stem cells: Embryonal stem cells can be maintained undifferentiated in culture when the medium is supplemented with Leukemia Inhibitory Factor (LIF). It was found that TEPA can replace LIF in maintaining the undifferentiative phenotype of the cells.

Thus, embryonal stem cells were cultured for 3-4 days essentially as described in (66), in the presence of LIF (20-100 ng/ml) or TEPA (10-20 μM) and their differentiation compared to non-treated control cells.

TABLE 9
The effect of TEPA of embryonal stem cells

Effect on

| Compound added | Differentiation | Proliferation |
|---|---|---|
| Control | + | +/− |
| LIF | − | + |
| TEPA | − | + |

The results presented in Table 9, clearly indicate that TEPA exerts a similar effect on embryonal stem cells as it does for other cell types.

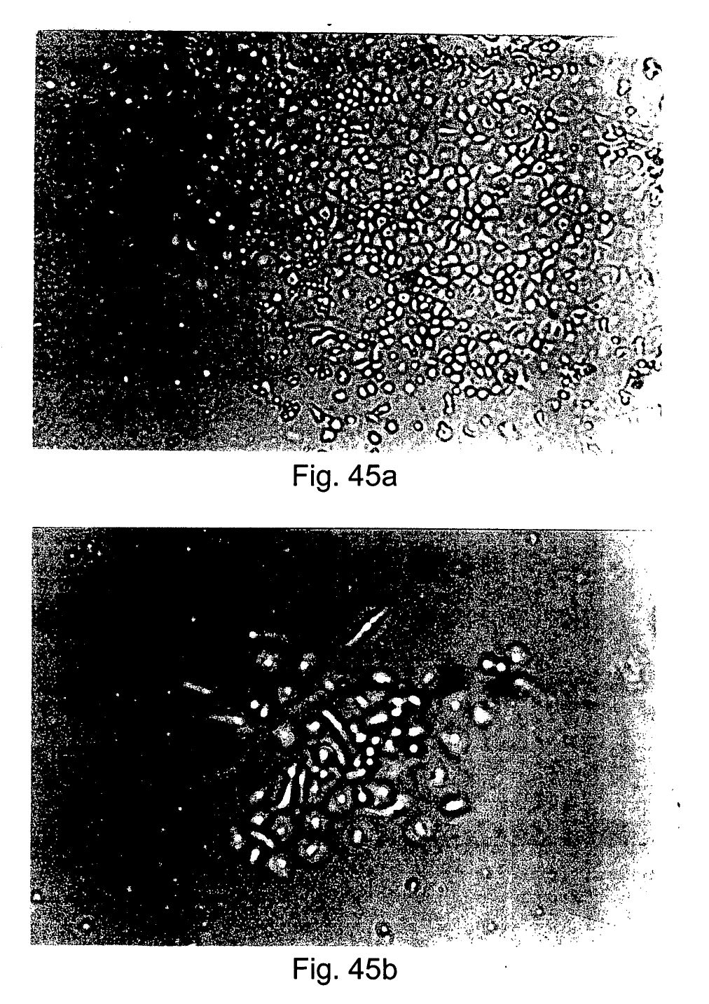

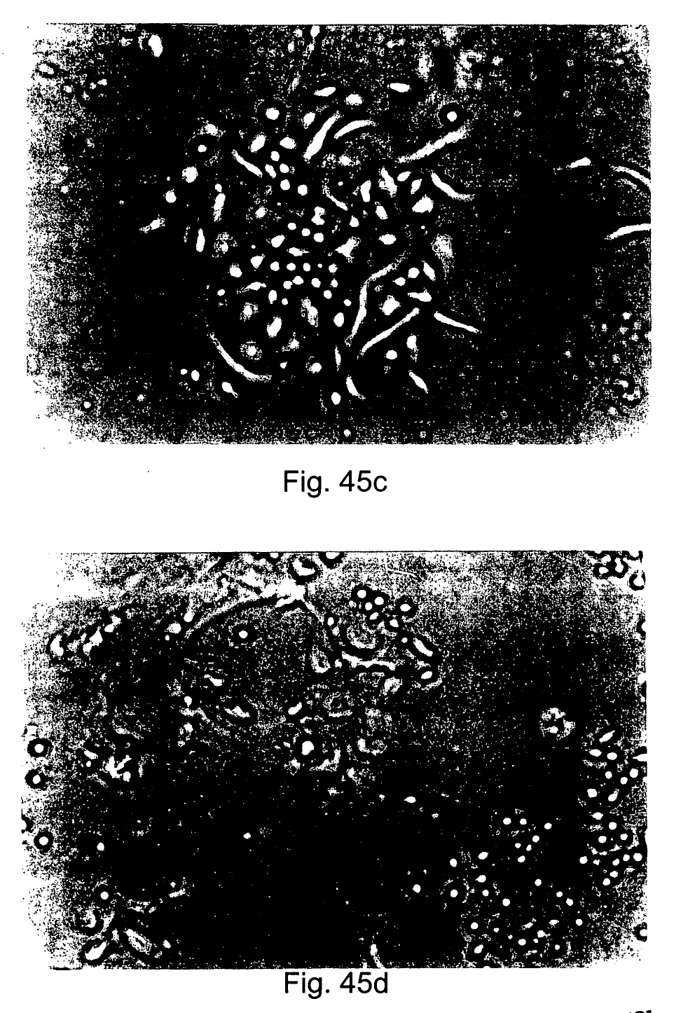

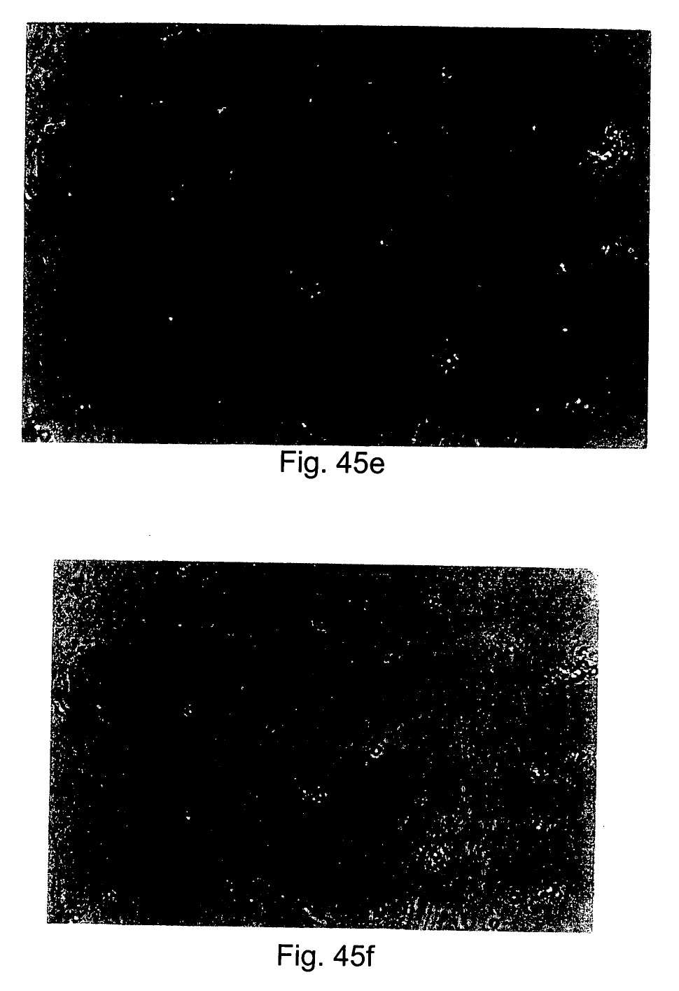

Hepatocytes: Livers were dissected from anesthetized BALB/c mice with sterile tools and immersed into F12 culture medium (Biological Industries, Kibbutz Bet Ha'Emek, Israel). The livers were washed three times with 3% BSA/PBS buffer and minced into small pieces with seizures. Following three washes with 3% BSA/PBS the liver tissue pieces were incubated for 30 minutes with 0.05% collagenase at 37° C. with continuos shaking under 5% CO₂ atmosphere. The digested liver tissue pieces were than mashed by pressing through a fine mesh strainer. After three washes with 3% BSA/PBS the liver cells were seeded into F12 culture medium enriched with: 15 mM HEPES buffer, 0.1 glucose, 10 mM sodium bicarbonate, 0.5 u/ml insulin, 7.5 ng/ml hydrocortisone and with or without 15 μg/ml of TEPA and incubated at 37° C. in a 5% CO₂ atmosphere. After overnight incubation the medium was removed and the cells were supplemented with fresh enriched F12 medium as described above with or without 15 μg/ml of TEPA. Hepatocytes were incubated in 35 mm dishes for several weeks with enriched F12 culture medium with or without 15 μg/ml TEPA at 37° C. under 5% CO₂ atmosphere. Cell culture medium was replaced every week with a fresh medium. Hepatocytes cultures that were ex-vivo expanded with TEPA for five weeks contained many dividing and undifferentiated cells (FIGS. 45a-d), while cultures that were not treated with TEPA contained a very small amount of only differentiated cells (FIGS. 45e-f).

Plant cells: The effect of TEPA on the intracellular copper content of plant cells was determined as follows. Boston fern Callus tissue cultures were obtained from a commercial plant tissue culture production facility (Biological Industries, Kibbutz Bet Ha'Emek, Israel) and incubated with different concentrations of TEPA in the culture medium for two days at room temperature. After three washes with PBS the tissues were suspended in 0.03 M ultra pure nitric acid and homogenized with a high shear mixer (polytron, Kinematica, Switzerland) for 3 minutes to disrupt the cells and release intracellular copper. Cell samples were vortexed before transferring to a vial autosampler and analyzed in duplicate by a Perkin Elmer graphite furnace atomic absorption spectrophotometer at a wavelength of 324.7 nm. The samples were analyzed against copper standard solution prepared from a commercial stock solution that was diluted with 0.03 M ultra pure nitric acid.

Table 10 below summarizes the effect of different TEPA concentration in the growth medium on the intracellular copper concentration of plant cells.

TABLE 10
Effect of different TEPA concentration
in the growth medium on the intracellular copper
concentration of plant callus tissues

| | Average Intracellular Copper Content |
|---|---|
| TEPA Concentration in Medium | (ppb) |
| 0 μM (Control) | 36.85 +/− 16.0 |
| 10 μM | 13.85 +/− 4.09 |
| 50 μM | 8.45 +/− 0.05 |
| 100 μM | 7.1 +/− 2.12 |

It is evident from Table 10 above that incubation of plant cells with TEPA causes a reduction of the intracellular content of copper in the cells.

### Example 10

### Evaluation of the in-vivo Potential of ex-vivo Cultured Cells

Engraftment of SCID mice by ex-vivo expanded human hematopoietic cells: Cord blood purified CD₃₄₊ cells either fresh or following 2 or 4 weeks of ex-vivo culture (plus or minus TEPA) were injected into NOD/SCID mice essentially as described in (56). After 4 weeks, the mice were sacrificed and their femora and tibias were excised and the bone marrow flushed with a syringe fitted with a 25 gauge needle. A single cell suspension was prepared, the cells were washed and an aliquot counted with Trypan blue.

In order to quantitate engrafted cells of human origin, cells were stained with FITC-conjugated anti CD₄₅ antibodies and PE-conjugated either anti CD₃₄, CD₁₉ or CD₃₃ antibodies. Anti CD₄₅ antibodies recognize human, but not mouse, cells, and thus, indicates the human origin of the cells.

The proliferation and differentiation potential of the engrafted cells was assayed by cloning bone marrow cells in semi-solid medium under conditions that allow specifically growth of human derived colonies essentially as described in (56).

The results (Table 11) indicate that the engraftment potential of expanded cells is higher than that of fresh cells, 20-60% CD₄₅₊ as compared to 3-6% CD₄₅₊ cells, respectively. All 6 cord blood samples that were expanded ex-vivo in the presence of TEPA succeeded to engraft the animals, whereas only 2 out of 6 samples that were expanded without TEPA engrafted.

TABLE 11

| | Ex-vivo | | ⁺Engraftment | | | |
|---|---|---|---|---|---|---|
| | Weeks | Treatment | CD₄₅ (%) | CD₃₄ (%) | CD₁₉ (%) | *Colonies |
| CB 2 | 0 | — | 4 | 1.6 | 1.7 | 100 |
| 10% FCS | | | | | | |
| | 2 | Cytokines | 40 | 11 | 15 | 260 |
| | 2 | TEPA + Cytokines | 56 | 13 | 11 | 330 |
| CB 2 | 0 | — | 3 | 1.2 | 1.5 | 70 |
| 10% FCS | | | | | | |
| CB 2 | 2 | Cytokines | 38 | 5.7 | 14 | 127 |
| 10% FCS | | | | | | |
| CB 2 | 2 | TEPA + Cytokines | 48 | 13.5 | 9 | 528 |
| 10% FCS | | | | | | |
| CB 3 | 0 | — | 4 | 1.8 | 2.2 | 250 |
| 10% FCS | | | | | | |
| CB 3 | 2 | Cytokines | 0 | 0 | 0 | 0 |
| 10% FCS | | | | | | |
| CB 3 | 2 | TEPA + Cytokines | 20 | 4 | 5 | 100 |
| 10% FCS | | | | | | |
| CB 4 | 2 | Cytokines | 5 | 1 | 0.7 | 4 |
| 1% FCS | | | | | | |
| CB 4 | 2 | TEPA + Cytokines | 28 | 7 | 8 | 185 |
| 1% FCS | | | | | | |
| CB 4 | 4 | Cytokines | 4 | 2 | 3 | 4 |
| 1% FCS | | | | | | |
| CB 4 | 4 | TEPA + Cytokines | 40 | 9 | 14 | 267 |
| 1% FCS | | | | | | |
| CB 5- | 4 | Cytokines | 4.7 | 1.6 | 1 | 5 |
| FCS | | | | | | |
| CB 5- | 4 | TEPA + Cytokines | 21 | 6 | 9 | 275 |
| FCS | | | | | | |
| CB 6 | 2 | Cytokines | died | died | died | died |
| 10% FCS | | | | | | |
| CB 6 | 2 | TEPA + Cytokines | 73 | 9 | 26 | 420 |
| 10% FCS | | | | | | |
| CB 6 | 2 | Cytokines | 6 | 4 | 6 | 8 |
| 1% FCS | | | | | | |
| CB 6 | 2 | TEPA + Cytokines | 73 | 16 | 19 | 350 |
| 1% FCS | | | | | | |

No. of cells transplanted per mouse:
Fresh CB = 1 × 10⁵ purified CD₃₄₊ cells;
Ex-vivo expanded = the yield of 1 × 10⁵ (CB1-4,6) or 0.5 × 10⁵ (CB5) cultured CB CD₃₄⁺ cells.
*No. of colonies (erythroid and myeloid) per 2 × 10⁵ SCID BM cells.
# Human neonatal cord blood.
⁺Mean of 2-3 mice.

Hematopoietic reconstitution of lethally irradiated mice—fresh vs. ex-vivo expanded cells: Three month old female Balb/c X C57B1/6 F1 mice were lethally irradiated (1000 rad) and transplanted one day later with 1×10⁵ fresh bone marrow cells or the yield of 1×10⁵ bone marrow cells expanded ex-vivo either with or without TEPA for 3 to 5 weeks, as detailed in Table 12. Peripheral blood WBC counts were performed on weekly basis.

The results indicated that WBC recovery was faster in mice transplanted with bone marrow cells expanded ex-vivo in the presence of TEPA as compared to fresh or cells expanded without TEPA.

TABLE 12

| | | | WBC × 10⁶/ml | | | Survival | | |
|---|---|---|---|---|---|---|---|---|
| | | | | Ex-vivo expansion | | | Ex-vivo expansion | |
| | | | | with | | | with | |
| | Ex-vivo | In-vivo | | | TEPA + | | | TEPA + |
| | (Weeks) | (Days) | Fresh BM | cytokines | cytokines | Fresh BM | cytokines | cytokines |
| Exp. I | 3 | 13 | 0.3 | 0.5 | 0.7 | 4/5 | 4/5 | 5/5 |
| | 3 | 19 | 0.48 | 0.58 | 1.5 | 4/5 | 4/5 | 5/5 |
| Exp. II | 3 | 11 | 0.17 | 0.07 | 0.92 | 5/5 | 4/5 | 5/5 |
| | 3 | 29 | 5.6 | 0 | 10.8 | 5/5 | 0/5 | 5/5 |
| Exp. III | 5 | 6 | 0.1 | 0.02 | 0.69 | 5/5 | 5/5 | 5/5 |
| | 5 | 11 | 0.21 | 0.23 | 1.27 | 3/5 | 5/5 | 5/5 |
| | 5 | 19 | n.d. | n.d. | n.d. | 3/5 | 2/5 | 4/5 |
| | 5 | 27 | n.d. | n.d. | n.d. | 3/5 | 1/5 | 4/5 |

No. of cells transplanted per mouse:
Fresh BM = 1 × 10⁵ cells.
Ex-vivo expansion = the yield of 1 × 10⁵ cultured BM cells.

Survival of irradiated mice that were not transplanted was 0/5 in all three experiments.

### Example 11

### Co-treatment of Copper Chelation and RAR Antagonism Has No Additive or Synergic Effect on Stem Cell Expansion

CD34+ cell cultures were supplemented for three weeks with a combination of four cytokines: SCF, TPO, IL-6, and FLt3, with or without the following additives: TEPA 5 μM, RAR antagonist 10⁻⁵ M and TEPA 5 μM plus RAR antagonist 10⁻⁵ M. From week three onward, all cultures were supplemented only with cytokines. At week 7, the number of cells and of CD34+ cells were determined. Culture content of CD34+ cells was determined from a purified, re-selected fraction, using the MiniMACS CD34 progenitor cell isolation kit (Miltenyi Biotec). The re-selected cells were counted, given absolute numbers of CD34+ cells in the culture. Percentages of early CD34+ cell subsets, CD34⁺Lin−, were determined from the re-selected CD34+ cell fraction. Cells were dually stained with CD34PE and a mixture of FITC-conjugated antibodies against CD38, CD33, CD14, CD15, CD3, CD4, CD61, CD19 FITC for determination of CD34+Lin− cells.

The results obtained indicated that co-treatment of copper chelation and RAR antagonism had no additive or synergic effect on stem cell expansion, suggesting that copper chelation and RAR antagonism affect a single signaling pathway.

### Example 12

### Co-treatment of Copper Chelation and CD38 Inhibition Has No Additive or Synergic Effect on Stem Cell Expansion

CD34+ cell cultures were supplemented for three weeks with a combination of four cytokines: IL-3, TPO, IL-6, and FLt3, with or without the following additives: TEPA 10 μM, Nicotinamide 10 mM, and TEPA 10 μM plus Nicotinamide 10 mM. From week three onward all cultures were supplemented only with cytokines. At week 5, number of cells and of CD34+ cells were determined. Culture content of CD34+ cells was determined from a purified, re-selected fraction, using the MiniMACS CD34 progenitor cell isolation kit (Miltenyi Biotec). The re-selected cells were counted, given absolute numbers of CD34+ cells in the culture.

The results obtained indicated that co-treatment of copper chelation and CD38 inhibition had no additive or synergic effect on stem cell expansion, suggesting that copper chelation and CD38 inhibition affect a single signaling pathway.

Hence, as is evident from Examples 11 and 12, combining different reagents, active at different cellular targets, demonstrated neither additive nor synergistic effect. These results support CD38 protein and it's biological function as a casual event in regulation of stem cells self-renewal.

### Example 13

### Inhibition of PI 3-kinase Results in Stem Cell Expansion

Experimental Procedures:

CD133+Cells: While CD34+ enriched cord or peripheral white blood cells have traditionally constituted a reference population enriched in undifferentiated hematopoietic cells for transplantation, the recent identification and isolation of human hematopoietic cells expressing additional markers such as CD133 (formerly known as AC133), has provided novel insights into the hematopoietic progenitor and stem cell compartment in the human, and a better understanding of the relationships between the cell surface phenotype of the subpopulations comprising the human hematopoietic system and their proliferative and differentiative capacity (see, for example, Bhatia, M., Leukemia 2001; 15:1685-88). Studies of cultures of the AC 133+ subpopulation indicate that CD133+ cells have high self-renewal capability, maintain early hematopoietic stem/progenitor cell (HSPC) characteristics, and show superior survival in culture, as compared to CD34+ cells (see Forraz, et al, Br. J. Haematology, 2002; 119:516-24). Applicability of the methods of the present invention for expansion and inhibition of differentiation of cells for transplantation to a broad range of undifferentiated hematopoietic stem/progenitor cells can be assessed using CD133+ cells as well. CD133+ cells can be identified and isolated for culture or cytometry using procedures well known in the art, such as the CliniMACS system, optimized for CD133 with CD133 MicroBeads (Miltenyi Biotech), or anti-CD133 monoclonal antibodies (cat no: 16-1331; eBioscience San Diego Calif., USA).

Mononuclear cell fraction collection and purification: Human blood cells were obtained from umbilical cord blood from female patients following full-term, normal delivery (informed consent was obtained). Samples were collected and processed within 12 hours postpartum. Blood was mixed with 3% Gelatin (Sigma, St. Louis, Mo.), sedimented for 30 minutes to remove most red blood cells. The leukocyte-rich fraction was harvested and layered on a Ficoll-Hypaque gradient (1.077 gram/ml; Sigma), and centrifuged at 400 g for 30 minutes. The mononuclear cell fraction in the interface layer was collected, washed three times and resuspended in phosphate-buffered saline (PBS) solution (Biological Industries) containing 0.5% bovine serum albumin (BSA, Fraction V; Sigma, St. Louis, Mo.).

Purification of CD34⁺ cells from mononuclear cell fractions: To purify CD34⁺ mononuclear cells, the mononuclear cell fraction was subjected to two cycles of immuno-magnetic separation using the MiniMACS® or Clinimax® CD34 Progenitor Cell Isolation Kit (Miltenyi Biotec, Auburn, Calif.) as per manufacturer's recommendations. The purity of the CD34⁺ population obtained ranged from 95% to 98%, as determined by flow cytometry (see below).

To further purify the CD34⁺ population into CD34⁺38⁻ or the CD34⁺Lin⁻ sub-fractions, the purified CD34⁺ cells were further labeled using monoclonal antibodies specific for CD38 (Dako A/S, Glostrup, Denmark) or lineage antigens (BD Biosciences, Erermbodegem, Belgium). The negatively labeled fraction (CD34⁺CD38⁻ or CD34⁺ Lin⁻) was measured and sorted by a FACS sorter.

For CD34⁻Lin⁻ purification, the CD34⁻ fraction was depleted from cells expressing lineage antigens using a negative selection column (StemCell Technologies, Vancouver, BC, Canada).

Ex vivo expansion: Purified CD34+ cells were cultured in culture bags (American Fluoroseal Co. Gaithersburg, Md., USA) at a concentration of 1×10⁴ cells/ml in MEMα/10% FCS containing the following human recombinant cytokines: Thrombopoietin (TPO), interleukin-6 (IL-6), FLT-3 ligand and stem cell factor (SCF), IL-3, each at a final concentration of 50 ng/ml (Perpo Tech, Inc., Rocky Hill, N.J., USA), with or without the PI 3-kinase inhibitor, Ly294002 at 0.1, 0.5, 1, 5, 10, 20, 50, 100 μM/L and incubated at 37° C. in a humidified atmosphere of 5% CO₂ in air. The cultures were replenished weekly, for three weeks, with the same volume of fresh medium, Ly294002 and growth factors. From week three and up to the termination of the experiments the cultures were weekly demi-depopulated. Cells were counted following staining with trypan blue. At various time points, harvested cells were used to assay the content of colony forming units in culture (CFUc), enumeration of CD34+ cells following re-selection and immunophenotype analysis. Cell morphology was determined on cytospin (Shandon, Pittsburgh, Pa., USA) prepared smears stained with May-Grunwald/Giemsa solutions.

Surface antigen analysis: The cells were washed with a PBS solution containing 1% BSA, and stained (at 4° C. for 30 min) with fluorescein isothiocyanate (FITC)- or phycoerythrin (PE)-conjugated antibodies. The cells were then washed in the above buffer and analyzed using a FACScalibur® flow cytometer (Becton Dickinson, San Jose, Calif., USA). The cells were passed at a rate of up to 1000 cells/second, using a 488 nm argon laser beam as the light source for excitation. Emission of 10⁴ cells was measured using logarithmic amplification, and analyzed using the CellQuest software (Becton Dickinson).

Cells stained with FITC- and PE-conjugated isotype control antibodies were used to determine background fluorescence.

Determination of CD34+ cell content after expansion: The content of CD34+ cells was determined from a purified, re-selected fraction of expanded hematopoietic progenitor cells, using the MiniMACS CD34 progenitor cell isolation kit (Miltenyi Biotec) according to the manufacturers recommendations. In brief, mononuclear cells derived from one portion of the culture were subjected to two cycles of immunomagnetic bead separation. The purity of the CD34+ population thus obtained was 95-98%, as evaluated by flow cytometry. CD34+ cell content of the entire culture was calculated as follows: number of CD34+ cells recovered following repurification after expansion multiplied by the culture volume/volume of the portion of the culture subjected to repurification (CD34+ₜₒₜₐₗ=CD34+ in sample×total culture volume/volume of repurified sample). Up to week three the cultures were topped weekly with fresh medium. Therefore, the culture volume was measured directly. From week three and on (due to demi-depopulation), the culture volume was calculating by multiplying the actual volume with the number of passages. Fold expansion was calculated by dividing the CD34+ cell content of the culture by the number of inoculated CD34+ cells (Fold expansion=total CD34+ content/CD34+ inoculated). FACS analysis of cells from 8 week cultures showed that the forward light scatter (FSC-H) and side light scatter (SSC-H) of the repurified CD34+ cells were similar to those of the CD34+ cells before culture. Giemsa staining showed that the morphology of the cells was identical to that of freshly purified CD34+ cells (data not shown).

Determination of early CD34+ cell subsets: The percentages of the early CD34+ cell subsets were determined as well from the re-purified CD34+ cell fraction. Cells were dually stained with PE anti-CD34 and FITC anti-CD38 antibodies for determination of CD34+CD38− cells, and with PE anti-CD34 antibodies and a mixture of FITC-conjugated antibodies against differentiation antigens (CD38, CD33, CD14, CD15, CD3, CD61, CD19) for determination of CD34+Lin− cells. Antibodies to CD34, CD38 and CD61 were purchased from DAKO (Glostrup, Denmark) and antibodies to CD33, CD14, CD15, CD3 and CD19—from Becton Dickinson (San Jose, Calif.). FACS analysis results of the above subsets are expressed as percentage of CD34+ cells. The absolute number of CD34+CD38− and CD34+Lin− cells in the culture was calculated from the total number of CD34+ cells recovered following the re-purification step.

Morphological assessment: Morphological characterization of the resulting culture populations was performed using aliquots of cells deposited on glass slides via cytospin (Cytocentrifuge, Shandon, Runcorn, UK). Cells were fixed, stained with May-Grunwald/Giemsa stain and examined microscopically.

Assay for Colony Forming Cells (CFUc): Mononuclear cells were added, (1500 cells/three ml), to semisolid medium, containing methylcellulose (Sigma), 30% FCS, 1% bovine serum albumin (BSA), 1×10⁻⁵ M β-mercaptoethanol (Sigma, St Louis, Mo.), 1 mM glutamine (Biological Industries), 2 IU/ml erythropoietin (Eprex, Cilag AG Int., Schaffhausen, Switzerland), SCF and IL-3, both at 20 ng/ml, G-CSF and GM-CSF, both at 10 ng/ml (Perpo Tech) and 2 μM hemin (Sigma). Following stirring the mixture was divided into two 35 mm dishes. The dishes were incubated for 14 days at 37° C. in a humidified atmosphere of 5% CO₂ in air. At the end of the incubation period, myeloid and erythroid colonies were counted under an inverted microscope at 40× magnification. CFUc content of the expansion culture was calculated as follows: Total number of scored colonies per two dishes×total mononuclear cell number/1500. Up to week three total mononuclear cells were determined by multiplying the number of cells per ml by the culture volume. From week three and on, number of passages was also taken into account (due to demi-depopulation).

Results:

In order to determine the effect of inhibition of the PI 3-kinase signal pathway on differentiation and expansion of stem and early progeitor cells, hematopoietic stem cell cultures were established as described hereinabove: CD34+ cells were supplemented for three weeks with a cytokine cocktail, with and without the PI 3-kinase inhibitor, Ly294002. To determine the long-term potential for expansion following a brief exposure to the PI 3-kinase inhibitor, beginning from week three the cultures were supplemented with only cytokines. Early (CD34+CD38−, CD34+Lin−) and late (CD34+ CFUc) progenitor cells were analyzed two and three weeks after initiation of the experiment. Late progenitor cells were analyzed for the remainder of the experiment.

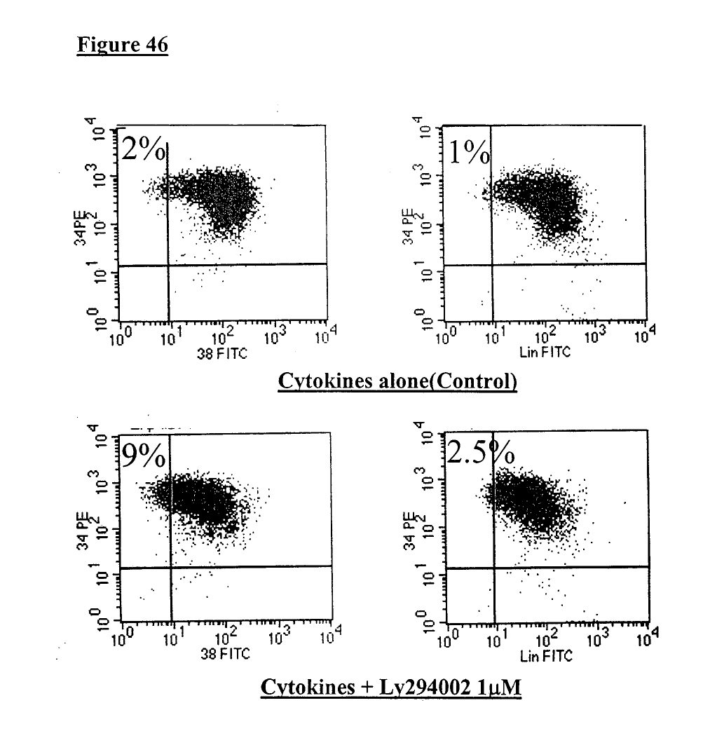

FACS analysis of cultures at two weeks (Table 13) demonstrates significantly higher percentages of early progenitor cell subsets, CD34+CD38− and CD34+Lin− cells in Ly294002-treated cultures (9.1% and 2.5%, respectively), compared with those in the control cultures (2.0 and 1.0, respectively). Representative FACS analysis of samples of Ly294002-treated and control cells with respect to CD34/CD38 and CD34/CD38/Lin is shown in FIG. 46. The absolute content of CD34+CD38− and CD34+CD38−Lin− cells was significantly higher in PI 3-kinase inhibitor-treated (9×10⁴ and 2.5×10⁴ cells respectively) than in control cultures (2×10⁴ and 1×10⁴ cells respectively). The content of late progenitor, CD34+ cells was similar in both treated (65×10⁴ cells) and untreated (55×10⁴ cells) cultures. However, the frequency of CD34+ cells among the total cultured cells was higher following two-weeks PI3-K-inhibitor treatment (0.3) than the frequency control cultures (0.2).

TABLE 13
The effect of LY294002 on ex vivo expansion of early and late
progenitor cells

| | | | CD34 + CD38- | CD34 + CD38- | CD34 + Lin- | CD34 + Lin- |
|---|---|---|---|---|---|---|
| | TNC × 10⁴ | CD34 × 10⁴ | (%) | ×10⁴ | (%) | ×10⁴ |
| Cytokines | 218 | 65 | 2.2 | 1.4 | 1.0 | 0.6 |
| Cytokines + | 155 | 53 | 9.1 | 4.8 | 2.5 | 1.3 |
| Ly294002 | | | | | | |

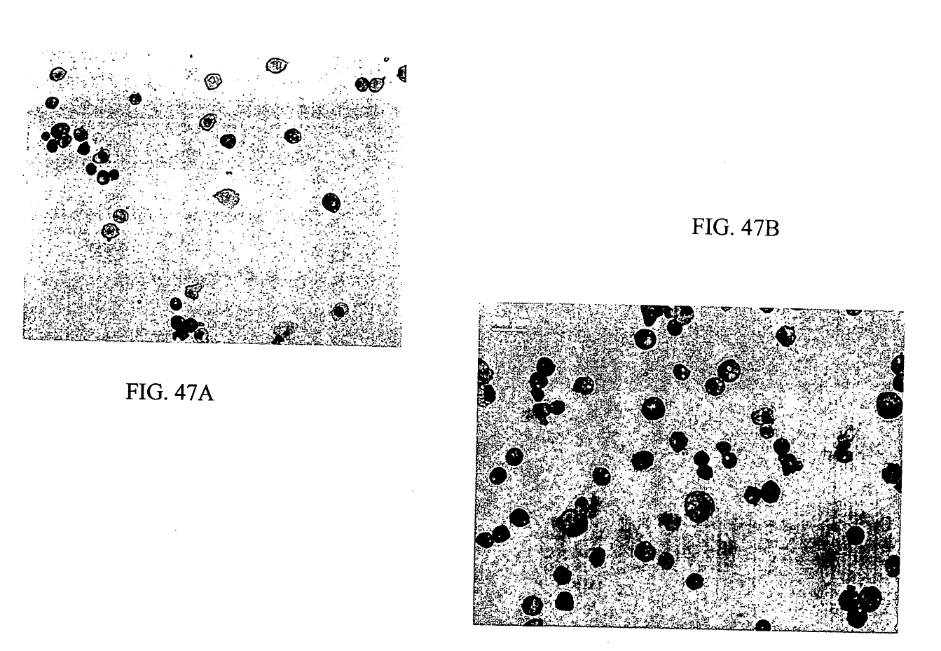

Morphological assessment of the cultured hematopoietic stem cells, after three weeks in culture, also clearly demonstrated the persistence of characteristic CD34⁺ cell morphology in cells exposed to LY294002, as compared with the macrophage-like appearance of the controls treated with cytokines alone (FIG. 47).

Taken together, these results show, for the first time, that inhibition of the PI 3-kinase signal pathway in stem cells, by a low molecular weight, specific inhibitor clearly enables self-renewal division of stem/early progenitor cells, as compared to cytokine treatment alone. Without wishing to be limited to a single hypothesis, one possible interpretation of these results is that PI 3-kinase is involved in the downstream signal transduction pathway governing hematopoietic cell differentiation. Therefore, inhibition of PI 3-kinase activity can allow expansion while inhibiting differentiation of stem- and early progenitor cells, such as those selected by CD34 and CD133 antibodies, and turn the balance of stem cell division toward stem cell expansion.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present invention.

### References Cited

### Additional References Are Cited in the Text

1. Kastner P, Mark M, Chambon P. Nonsteroid nuclear receptors: what are genetic studies telling us about their role in real life? Cell. 1995; 83:859-86
2. Mangelsdorf D, Evans R. The RXR and orphan receptors. Cell. 1995; 83:841-850
3. Minucci S. and Pelicci P. G. Retinoid receptors in health and disease: co-regulators and the chromatin connection. Semin Cell Dev Biol. 1999; 10(2):215-25.
4. Brand N, Petkovich M, Krust A, Chambon P, de The H, Marchio A, Tiollais P, Dejean A. Identification of a second human retinoic acid receptor. Nature, 1988; 332: 850-53
5. Ishikawa T, Umesono K, Mangelsdorf D J, Aburatani H, Stanger B Z, Shibasaki Y, Imawari M, Evans R M, Takaku F. A functional retinoic acid receptor encoded by the gene on human chromosome 12. Mol. Endocrin. 1990; 4: 837-44
6. Mangelsdorf D J, Ong E S, Dyck J A, Evans R M. Nuclear receptor that identifies a novel retinoic acid response pathway. Nature. 1990; 345: 224-29
7. Mangelsdorf D J, Borgmeyer U, Heyman R A, Zhou J Y, Ong E S, Oro A E, Kakizuka A, Evans R M. Characterization of three RXR genes that mediate the action of 9-cis retinoic acid. Genes and Devel. 1992; 6: 329-44
8. Mangelsdorf D J, Umesono K, Kliewer S A, Borgmeyer U, Ong E S, Evans R M. A direct repeat in the cellular retinol-binding protein type II gene confers differential regulation by RXR and RAR. Cell. 1991; 66(3):555-61.
9. Heyman R A, Mangelsdorf D J, Dyck J A, Stein R B, Eichele G, Evans R M, Thaller C. 9-cis retinoic acid is a high affinity ligand for the retinoid X receptor. Cell. 1992; 68: 397-406
10. Levin A A, Sturzenbecker L J, Kazmer S, Bosakowski T, Huselton C, Allenby G, Speck J, Kratzeisen C, Rosenberger M, Lovey A, et al. 9-cis retinoic acid stereoisomer binds and activates the nuclear receptor RXR alpha. Nature. 1992; 355: 359-61
11. Levin M. S., and Davis A. E. Retinoic acid increases cellular retinol-binding protein II mRNA and retinol uptake in the human intestinal Caco-2 cell line. J. Nutr. 1997; 127:13-17
12. Suruga K, Mochizuki K, Suzuki R, Goda T, and Takase S. Regulation of cellular retinol-binding protein type II gene expression by arachidonic acid analogue and 9-cis retinoic acid in Caco-2 cells. Eur. J. Biochem.1999; 262:70-78
13. Kliewer, S A., Umesono K, Noonan D J, Heyman R A, and Evans R M. Convergence of 9-cis retinoic acid and peroxisome proliferator signaling pathway through heterodimer formation of their receptors. Nature 1992; 358: 771-774.
14. Siegenthaler G. Extra- and intracellular transport of retinoids: a reappraisal. Horm Res. 1996; 45:122-127
15. Sherman M I, Gubler M L, Barkai U, Harper M I, Coppola G, Yuan J. Role of retinoids in differentiation and growth of embryonal carcinoma cells. Ciba Found Symp. 1985;1 13:42-60.
16. Herdick M, Steinmeyer A, Carlberg C. Carboxylic ester antagonists of 1alpha,25-dihydroxyvitamin D(3) show cell-specific actions. Chem Biol. 2000: 7:885-94.
17. Jimenez-Lara A M, Aranda A. Interaction of vitamin D and retinoid receptors on regulation of gene expression. Horm Res. 2000; 54:301-5
18. Evans R. The steroid and thyroid hormone receptor super family. Science. 1988;240:889-895.
19. Breitman T, Selonick S, Collins S. Induction of differentiation of the human promyelocytic leukemia cell line (HL-60) by retinoic acid. Proc Natl Acad Sci USA. 1980;77:2936-2940.
20. Purton L E, Bernstein I D, Collins S J. All-trans retinoic acid delays the differentiation of primitive hematopoietic precursors (lin c-kit+ Sca-1+) while enhancing the terminal maturation of committed granulocyte/monocyte progenitors. Blood. 1999;94:483-495.
21. Louise E. Purton, Irwin D. Bernstein, and Steven J. Collins, All-trans retinoic acid enhances the long-term repopulating activity of cultured hematopoietic stem cells. Blood. 2000; 95: 470-477
22. Muramatsu M, Hanazono Y, Ogasawara Y, Okada T, Mizukami H, Kume A, Mizoguchi H, Ozawa K. Reversible integration of the domain negative retinoid receptor gene for ex-vivo expansion of hematopoietic stem/progenitor cells. Biochem Biophys Res Commun 2001; 285:891-6
23. U.S. Pat. No. 5,830,760. creating novel hematopoietic cell lines by expressing altered retinoic acid receptors.
24. Kapil M., Teresa M., Taghi M., Michael A., Steven C., Maher A. Involvement of retinoic acid receptor mediated signaling pathway in induction of CD38 cell surface antigen, Blood. 1997; 89:3607-3614
25. Ueno H, Kizaki M, Matsushita H, Muto A, Yamato K, Nishihara T, Hida T, Yoshimura H, Koeffler H P, Ikeda Y. A novel retinoic acid receptor (RAR)-selective antagonist inhibits differentiation and apoptosis of HL-60 cells: implications of RAR alpha-mediated signals in myeloid leukemic cells. Leuk Res. 1998; 22:517-25
26. Johnson A T, Wang L, Standeven A M, Escobar M, Chandraratna R A. Synthesis and biological activity of high-affmity retinoic acid receptor antagonists. Bioorg Med Chem. 1999; 7(7):1321-38.
27. Endo Y. “Dicarba-closo-dodecaboranes as a pharmacophore. Retinidal antagonists and potential agonists”. Chem Pharm Bull. 1999; 47(3):398-404.
28. Drayson M T, Michell R H, Durham J, Brown G. Cell proliferation and CD11b expression are controlled independently during HL60 cell differentiation initiated by 1,25 alpha-dihydroxyvitamin D(3) or all-trans-retinoic acid. Exp Cell Res. 2001;266(l):126-34
29. Gudas, L J, Sporn, M B, & Roberts, A B. Cellular biology and biochemistry of the retinoids. In: The retinoids: biology, chemistry and medicine. 1994:443 516.
30. Wolbach, S B & Howe, P R. Tissue changes following deprivation of fat-soluble A vitamin. J Exp Med 1925, 42:753 777.
31. De The, H, Marchino, A, Tiollais, P, & Dejean, A. Differential expression and ligand regulation of the retinoic acid receptor α and β genes. EMBO J 1989, 8:429 433
32. De The, H, Vivanco Ruiz, M, Tiollais, P, Stunnenberg, H, & Dejean, A. Identification of retinoic responsive element in the retinoic acid receptor gene. Nature. 1990; 343:177 180.
33. Nervi, C, Vollberg, T M, George, M D, Zelent, A, Chambon, P, & Jetten, A M. Expression of nuclear retinoic acid receptors in normal tracheobronchial cells and in lung carcinoma cells. Exp Cell Res. 1991; 195:163 170.
34. Swisshelm, K, Ryan, K, Lee, X, Tsou, H C, Peacocke, M, and Sager, R. Down-regulation of retinoic acid receptor β in mammary carcinoma cell lines and its up-regulation in senescing normal mammary epithelial cells. Cell Growth Differ. 1994; 5:133 141
35. Caliaro, M J, Marmouget, C, & Guichard, S. Response of four ovarian carcinoma cell lines to all-trans retinoic acid: relationship with induction of differentiation and retinoic acid receptor expression. Int J Cancer 1994, 56:743 748.
36. Comerci, J T, Hallam, S, Goldberg, G L, Runowcz, C D, Fields, A L, Wadler, S, & Gallagher, R E. Expression of retinoic acid receptor-β2 mRNA in normal cervical epithelium and cervical squamous cell carcinoma. Int J Oncology 1997, 11:983 988.
37. Xu, X C, Ro, J Y, Lee, J S, Shin, D M, Hong, W K, and Lotan, R. Differential expression of nuclear retinoid receptors in normal, premalignant and malignant head and neck tissues. Cancer Res 1994; 54:3580 3587
38. Labrecque J., et al. Impaired granulocytic differentiation in vitro in hematopoietic cells lacking retinoic acid receptors alpha1 and gamma Blood 1998; 92: 607-615.
39. Douer D, Ramezani L, Parker J, Levine A M. All-trans-retinoic acid affects the growth, differentiation and apoptosis of normal human myeloid progenitors derived from purified CD34+ bone marrow cells. Leukemia. 2000;14:874-881.
40. Morosetti R, Grignani F, Liberatore C, et al. Infrequent alterations of the RAR gene in acute myelogenous leukemias, retinoic acid-resistant acute promyelocytic leukemias, myelodysplastic syndromes and cell lines. Blood 1996; 87:4399-4403.
41. Purton L E, Bernstein I D, and Collins S J. All-trans retinoic acid enhances the long-term repopulating activity of cultured hematopoietic stem cells. Blood. 2000; 95(2): 470-477.
42. Douer D, Ramezani L, Parker J, Levine A M. All-trans-retinoic acid affects the growth, differentiation and apoptosis of normal human myeloid progenitors derived from purified CD34+ bone marrow cells. Leukemia. 2000;14:874-881
43. Kamei Y, Kawada T, Mizukami J, Sugimoto E. The prevention of adipose differentiation of 3T3-L1 cells caused by retinoic acid is elicited through retinoic acid receptor alpha. Life Sci. 1994; 55(16):PL307-12
44. Van Epps D E, et al. Harvesting, characterization, and culture of CD34+ cells from human bone marrow, peripheral blood, and cord blood. Blood Cells 20:411, 1994.
45. Emerson S G. Ex-vivo expansion of hematopoietic precursors, progenitors, and stem cells: The next generation of cellular therapeutics. Blood 87:3082, 1996.
46. Brugger W, et al. Reconstitution of hematopoiesis after high-dose chematotherapy by autologous progenitor cells generated in-vivo. N Engl J Med 333:283, 1995.
47. Williams S F, et al. Selection and expansion of peripheral blood CD34+ cells in autologous stem cell transplantation for breast cancer. Blood 87:1687, 1996.
48. Zimmerman R M, et al. Large-scale selection of CD34+ peripheral blood progenitors and expansion of neutrophil precursors for clinical applications. J Heamatotherapy 5:247, 1996.
49. Koller M R, Emerson S G, Palsson B O. Large-scale expansion of human stem and progenitor cells from bone marrow mononuclear cells in continuous perfusion cultures. Blood 82:378, 1993.
50. Lebkowski J S, et al. Rapid isolation and serum-free expansion of human CD34+ cells. Blood Cells 20:404, 1994.
51. Sandstrom C E, et al. Effects of CD34+ cell selection and perfusion on ex-vivo expansion of peripheral blood mononuclear cells. Blood 86:958, 1995.
52. Palmiter R D. Regulation of metallothionein genes by heavy metals appears to be mediated by a zinc-sensitive inhibitor that interacts with a constitutively active transcription factor, MTF-1. Proc Natl Acad Sci USA 91(4): 1219-1223, 1994.
53. Freedman A R, et al. Generation of T lymphocytes from bone marrow CD34+ cells in-vitro. Nature Medicine 2: 46, 1996.
54. Heslop H E, et al. Long term restoration of immunity against Epstein-Barr virus infection by adoptive transfer of gene-modified virus-specific T lymphocytes. Nature Medicine 2: 551, 1996.
55. Protti M P, et al. Particulate naturally processed peptides prime a cytotoxic response against human melanoma in-vitro. Cancer Res 56: 1210, 1996.
56. Rosenberg S A, et al. Prospective randomized trial of high-dose interleukin-2 alone or in conjunction with lymphokine-activated killer cells for the treatment of patients with advanced cancer. J Natl Cancer Inst 85: 622, 1993.
57. Bernhard H, et al. Generation of immunostimulatory dendritic cells from human CD34+ hematopoietic progenitor cells of the bone marrow and peripheral blood. Cancer Res 10: 99, 1995.
58. Fisch P, et al. Generation of antigen-presenting cells for soluble protein antigens ex-vivo from peripheral blood CD34+ hematopoietic progenitor cells in cancer patients. Eur J Immunol 26: 595, 1996.
59. Siena S, et al. Massive ex-vivo generation of functional dendritic cells from mobilized CD34+ blood progenitors for anticancer therapy. Expt Hematol 23:1463, 1996.
60. Repair of myelin disease: Strategies and progress in animal models. Molecular Medicine Today. December 1997. pp. 554-561
61. Schechtez A N et al. Sickle cell anemia. In: Molecular basis of blood diseases. Stamatoyannaopoulos G, Nienhuis A W, Leder P and Majerus P W Eds. pp. 179-218, Sounders Philadelphia.
62. Alter B P. Fetal erythropoiesis in stress hemopoiesis. Experimental Hematology 7: 200, 1979.
63. Blau C A et al. Fetal hemoglobin in acute and chronic stage of erythroid expansion. Blood 81: 227, 1993.
64. Lala D S, Mukherjee R, Schulman I G, Koch S S, Dardashti L J, Nadzan A M, Croston G E, Evans R M, Heyman R A. Activation of specific RXR heterodimers by an antagonist of RXR homodimers. Nature. 1996; 383:450-3.
65. Kochhar D M, Jiang H, Penner J D, Johnson A T, Chandraratna R A. The use of a retinoid receptor antagonist in a new model to study vitamin A-dependent developmental events. Int. J. Dev. Biol. 1998;42(4):601-8
66. Li M, Chiba H, Warot X, Messaddeq N, Gerard C, Chambon P, Metzger D. RXR-alpha ablation in skin keratinocytes results in alopecia and epidermal alterations. Development. 2001;128(5):675-88.
67. Fibach E, Manor D, Oppenheim A, Rachmilewitz E A. Proliferation and maturation of human erythroid progenitors in liquid medium. Blood 73:100, 1989.
68. Fibach E, Manor D, Treves A, Rachmilewitz E A. Growth of human normal erythroid progenitors in liquid culture: A comparison with colony growth in semisolid culture. Internatl J Cell Clon 9:57, 1991.
69. Fibach E, Rachmilewitz E A. The two-step liquid culture—novel procedure for studying maturation of human normal and pathologic erythroid precursors. Stem Cells 11:36, 1993.
70. Dalyot N, Fibach E, Rachmilewitz E, Oppenheim A. Adult and neonatal patterns of human globin gene expression are recapitulated in liquid cultures. Exper Hematol 20:1141, 1992.

1. A method of ex vivo expanding and inhibiting differentiation of a population of stem cells, the method comprising:
(a) providing the cells ex vivo with conditions for cell proliferation;
(b) ex vivo providing the cells with an effective concentration of a modulator of PI 3-kinase activity, said modulator selected capable of downregulating a PI 3-kinase activity or an expression of a gene encoding a PI 3-kinase;
thereby ex vivo expanding and inhibiting differentiation of the population of stem cells.

2. The method of claim 1, wherein said stem cells are early hematopoietic and/or hematopoietic progenitor cells.

3. The method of claim 1, wherein said modulator selected capable of downregulating a PI 3-kinase activity or an expression of a gene encoding a PI 3-kinase is selected from the group consisting of:
(a) an inhibitor of PI 3-kinase catalytic activity;
(b) an antisense polynucleotide capable of specifically hybridizing with an mRNA transcript encoding a PI 3-kinase;
(c) a ribozyme which specifically cleaves PI 3-kinase transcripts, coding sequences and/or promoter elements;
(d) an siRNA molecule capable of inducing degradation of PI 3-kinase transcripts; and
(e) a DNAzyme which specifically cleaves PI 3-kinase transcripts or DNA.

4. The method of claim 3, wherein said inhibitor of PI 3-kinase activity is Wortmannin or LY294002.

5. The method of claim 1, wherein said modulator capable of downregulating PI 3-kinase activity or expression of a gene encoding PI 3-kinase is an anti-PI 3-kinase antibody.

6. The method of claim 5, wherein said anti-PI 3-kinase antibody is ScFV or Fab.

7. The method of claim 3, wherein said providing is effected by transiently expressing said antisense polynucleotide, said ribozyme, said siRNA molecule or said DNAzyme within a stem cell.

8. The method of claim 3, wherein said providing is effected by
(a) providing an expressible polynucleotide capable of expressing said antisense polynucleotide, said ribozyme, said siRNA molecule or said DNAzyme, and
(b) stably integrating said expressible polynucleotide into a genome of a cell,
thereby providing a modulator capable of downregulating a PI 3-kinase activity or PI 3-kinase gene expression.

9. The method of claim 3, wherein said inhibitor of PI 3-kinase activity is an expressible polynucleotide encoding an anti-PI 3-kinase ScFv or Fab.

10. The method of claim 1, wherein said providing said conditions for cell proliferation is effected by providing the cells with nutrients and cytokines.

11. The method of claim 10, wherein said cytokines are selected from the group consisting of early acting cytokines and late acting cytokines.

12. The method of claim 11, wherein said early acting cytokines are selected from the group consisting of stem cell factor, FLT3 ligand, interleukin-6, thrombopoietin and interleukin-3.

13. The method of claim 11, wherein said early acting cytokine is FLT3 ligand.

14. The method of claim 11, wherein said late acting cytokines are selected from the group consisting of granulocyte colony stimulating factor, granulocyte/macrophage colony stimulating factor and erythropoietin.

15. The method of claim 11, wherein said late acting cytokine is granulocyte colony stimulating factor.

16. The method of claim 1, wherein said stem cells are derived from a source selected from the group consisting of hematopoietic cells, neural cells, oligodendrocyte cells, skin cells, hepatic cells, embryonal stem cells, muscle cells, bone cells, mesenchymal cells, pancreatic cells, chondrocytes and stroma cells.

17. The method of claim 16, wherein said stem cells are derived from bone marrow or peripheral blood.

18. The method of claim 16, wherein said stem cells are derived from neonatal umbilical cord blood.

19. The method of claim 1, further comprising the step of selecting a population of stem cells enriched for hematopoietic stem cells.

20. The method of claim 19, wherein said selection is affected via CD34.

21. The method of claim 1, further comprising the step of selecting a population of stem cells enriched for early hematopoietic stem/progenitor cells.

22. The method of claim 21, wherein said selection is affected via CD133.

23. A method of transducing expanded, undifferentiated stem cells with an exogene, the method comprising:
(a) obtaining a population of stem cells;
(b) expanding and inhibiting differentiation of said stem cells by:
(i) providing said stem cells with conditions for cell proliferation;
(ii) providing said stem cells with an effective concentration of a modulator of PI 3-kinase activity, said modulator selected capable of downregulating a PI 3-kinase activity or an expression of a gene encoding a PI 3-kinase;
wherein steps (i) and (ii) are effected in vitro or ex vivo, thereby expanding and inhibiting differentiation of said stem cells; and
(c) transducing said expanded, undifferentiated stem cells with the exogene.

24. The method of claim 23, wherein said transducing is effected by a vector including the exogene.

25. The method of claim 23, wherein said stem cells are early hematopoietic and/or hematopoietic progenitor cells.

26. The method of claim 23, wherein said modulator selected capable of downregulating a PI 3-kinase activity or an expression of a gene encoding a PI 3-kinase is selected from the group consisting of:
(a) an inhibitor of PI 3-kinase catalytic activity;
(b) an antisense polynucleotide capable of specifically hybridizing with an mRNA transcript encoding a PI 3-kinase;
(c) a ribozyme which specifically cleaves PI 3-kinase transcripts, coding sequences and/or promoter elements;
(d) an siRNA molecule capable of inducing degradation of PI 3-kinase transcripts; and
(e) a DNAzyme which specifically cleaves PI 3-kinase transcripts or DNA.

27. The method of claim 26, wherein said inhibitor of PI 3-kinase activity is Wortmannin or LY294002.

28. The method of claim 23, wherein said modulator capable of downregulating PI 3-kinase activity or expression of a gene encoding PI 3-kinase is an anti-PI 3-kinase antibody.

29. The method of claim 28, wherein said anti-PI 3-kinase antibody is ScFV or Fab.

30. The method of claim 26, wherein said providing is effected by transiently expressing said antisense polynucleotide, said ribozyme, said siRNA molecule or said DNAzyme within a stem cell.

31. The method of claim 26, wherein said providing is effected by
(a) providing an expressible polynucleotide capable of expressing said antisense polynucleotide, said ribozyme, said siRNA molecule or said DNAzyme, and
(b) stably integrating said expressible polynucleotide into a genome of a cell,
thereby providing a modulator capable of downregulating a PI 3-kinase activity or PI 3-kinase gene expression.

32. The method of claim 26, wherein said inhibitor of PI 3-kinase activity is an expressible polynucleotide encoding an anti-PI 3-kinase ScFv or Fab.

33. The method of claim 23, wherein said providing said conditions for cell proliferation is effected by providing the cells with nutrients and cytokines.

34. The method of claim 33, wherein said cytokines are selected from the group consisting of early acting cytokines and late acting cytokines.

35. The method of claim 34, wherein said early acting cytokines are selected from the group consisting of stem cell factor, FLT3 ligand, interleukin-6, thrombopoietin and interleukin-3.

36. The method of claim 34, wherein said early acting cytokine is FLT3 ligand.

37. The method of claim 34, wherein said late acting cytokines are selected from the group consisting of granulocyte colony stimulating factor, granulocyte/macrophage colony stimulating factor and erythropoietin.

38. The method of claim 34, wherein said late acting cytokine is granulocyte colony stimulating factor.

39. The method of claim 23, wherein said stem cells are derived from a source selected from the group consisting of hematopoietic cells, neural cells, oligodendrocyte cells, skin cells, hepatic cells, embryonal stem cells, muscle cells, bone cells, mesenchymal cells, pancreatic cells, chondrocytes and stroma cells.

40. The method of claim 39, wherein said stem cells are derived from bone marrow or peripheral blood.

41. The method of claim 39, wherein said stem cells are derived from neonatal umbilical cord blood.

42. The method of claim 23, further comprising the step of selecting a population of stem cells enriched for hematopoietic stem cells.

43. The method of claim 42, wherein said selection is affected via CD34.

44. The method of claim 23, further comprising the step of selecting a population of stem cells enriched for early hematopoietic stem/progenitor cells.

45. The method of claim 44, wherein said selection is affected via CD133.

46. A therapeutic ex vivo cultured stem cell population comprising undifferentiated hematopoietic cells expanded according to the methods of any of claims 1-45.

47. The cell population of claim 46, in a culture medium comprising a modulator of PI 3-kinase activity, said modulator selected capable of downregulating a PI 3-kinase activity or an expression of a gene encoding a PI 3-kinase.

48. The cell population of claim 47, isolated from said medium.

49. A pharmaceutical composition comprising the cell population of claim 46 and a pharmaceutically acceptable carrier.

50. A pharmaceutical composition comprising the cell population of claim 48 and a pharmaceutically acceptable carrier.

51. A method of hematopoietic stem cells transplantation into a recipient, the method comprising:
(a) obtaining a population of hematopoietic stem cells;
(b) ex vivo expanding and inhibiting differentiation of said hematopoietic stem cells by:
(i) ex vivo providing said hematopoietic stem cells with conditions for cell proliferation;
(ii) providing said hematopoietic stem cells ex vivo with an effective concentration of a modulator of PI 3-kinase activity, said modulator selected capable of downregulating a PI 3-kinase activity or an expression of a gene encoding a PI 3-kinase;
thereby expanding and inhibiting differentiation of said stem hematopoietic cells; and
(c) transplanting said hematopoietic stem cells into the recipient.

52. The method of claim 51, wherein said hematopoietic stem cells are early hematopoietic and/or hematopoietic progenitor cells.

53. The method of claim 51, wherein said modulator selected capable of downregulating a PI 3-kinase activity or an expression of a gene encoding a PI 3-kinase is selected from the group consisting of:
(a) an inhibitor of PI 3-kinase catalytic activity;
(b) an antisense polynucleotide capable of specifically hybridizing with an mRNA transcript encoding a PI 3-kinase;
(c) a ribozyme which specifically cleaves PI 3-kinase transcripts, coding sequences and/or promoter elements;
(d) an siRNA molecule capable of inducing degradation of PI 3-kinase transcripts; and
(e) a DNAzyme which specifically cleaves PI 3-kinase transcripts or DNA.

54. The method of claim 53, wherein said inhibitor of PI 3-kinase activity is Wortmannin or LY294002.

55. The method of claim 51, wherein said modulator capable of downregulating PI 3-kinase activity or expression of a gene encoding PI 3-kinase is an anti-PI 3-kinase antibody.

56. The method of claim 55, wherein said anti-PI 3-kinase antibody is ScFV or Fab.

57. The method of claim 53, wherein said providing is effected by transiently expressing said antisense polynucleotide, said ribozyme, said siRNA molecule or said DNAzyme within a stem cell.

58. The method of claim 53, wherein said providing is effected by
(a) providing an expressible polynucleotide capable of expressing said antisense polynucleotide, said ribozyme, said siRNA molecule or said DNAzyme, and
(b) stably integrating said expressible polynucleotide into a genome of a cell,
thereby providing a modulator capable of downregulating a PI 3-kinase activity or PI 3-kinase gene expression.

59. The method of claim 53, wherein said inhibitor of PI 3-kinase activity is an expressible polynucleotide encoding an anti-PI 3-kinase ScFv or Fab.

60. The method of claim 51, wherein said providing said conditions for cell proliferation is effected by providing the cells with nutrients and cytokines.

61. The method of claim 60, wherein said cytokines are selected from the group consisting of early acting cytokines and late acting cytokines.

62. The method of claim 61, wherein said early acting cytokines are selected from the group consisting of stem cell factor, FLT3 ligand, interleukin-6, thrombopoietin and interleukin-3.

63. The method of claim 61, wherein said early acting cytokine is FLT3 ligand.

64. The method of claim 61, wherein said late acting cytokines are selected from the group consisting of granulocyte colony stimulating factor, granulocyte/macrophage colony stimulating factor and erythropoietin.

65. The method of claim 61, wherein said late acting cytokine is granulocyte colony stimulating factor.

66. The method of claim 51, wherein said stem cells are derived from a source selected from the group consisting of hematopoietic cells, neural cells, oligodendrocyte cells, skin cells, hepatic cells, embryonal stem cells, muscle cells, bone cells, mesenchymal cells, pancreatic cells, chondrocytes and stroma cells.

67. The method of claim 66, wherein said stem cells are derived from bone marrow or peripheral blood.

68. The method of claim 66, wherein said stem cells are derived from neonatal umbilical cord blood.

69. The method of claim 51, further comprising the step of selecting a population of stem cells enriched for hematopoietic stem cells.

70. The method of claim 69, wherein said selection is affected via CD34.

71. The method of claim 51, further comprising the step of selecting a population of stem cells enriched for early hematopoietic stem/progenitor cells.

72. The method of claim 71, wherein said selection is affected via CD133.

73. A method of adoptive immunotherapy comprising:
(a) obtaining progenitor hematopoietic cells from a patient;
(b) ex vivo expanding and inhibiting differentiation of said hematopoietic cells by:
(i) providing said progenitor hematopoietic cells ex vivo with conditions for cell proliferation;
(ii) providing said progenitor hematopoietic cells with an effective concentration of a modulator of PI 3-kinase activity, said modulator selected capable of downregulating a PI 3-kinase activity or an expression of a gene encoding a PI 3-kinase;
thereby expanding and inhibiting differentiation of said progenitor hematopoietic cells; and
(c) transplanting said progenitor hematopoietic cells into a recipient.

74. The method of claim 73, wherein said hematopoietic stem cells are early hematopoietic and/or hematopoietic progenitor cells.

75. The method of claim 73, wherein said modulator selected capable of downregulating a PI 3-kinase activity or an expression of a gene encoding a PI 3-kinase is selected from the group consisting of:
(a) an inhibitor of PI 3-kinase catalytic activity;
(b) an antisense polynucleotide capable of specifically hybridizing with an mRNA transcript encoding a PI 3-kinase;
(c) a ribozyme which specifically cleaves PI 3-kinase transcripts, coding sequences and/or promoter elements;
(d) an siRNA molecule capable of inducing degradation of PI 3-kinase transcripts; and
(e) a DNAzyme which specifically cleaves PI 3-kinase transcripts or DNA.

76. The method of claim 75, wherein said inhibitor of PI 3-kinase activity is Wortmannin or LY294002.

77. The method of claim 73, wherein said modulator capable of downregulating PI 3-kinase activity or expression of a gene encoding PI 3-kinase is an anti-PI 3-kinase antibody.

78. The method of claim 77, wherein said anti-PI 3-kinase antibody is ScFV or Fab.

79. The method of claim 75, wherein said providing is effected by transiently expressing said antisense polynucleotide, said ribozyme, said siRNA molecule or said DNAzyme within a stem cell.

80. The method of claim 75, wherein said providing is effected by
(a) providing an expressible polynucleotide capable of expressing said antisense polynucleotide, said ribozyme, said siRNA molecule or said DNAzyme, and
(b) stably integrating said expressible polynucleotide into a genome of a cell,
thereby providing a modulator capable of downregulating a PI 3-kinase activity or PI 3-kinase gene expression.

81. The method of claim 75, wherein said inhibitor of PI 3-kinase activity is an expressible polynucleotide encoding an anti-PI 3-kinase ScFv or Fab.

82. The method of claim 73, wherein said providing said conditions for cell proliferation is effected by providing the cells with nutrients and cytokines.

83. The method of claim 82, wherein said cytokines are selected from the group consisting of early acting cytokines and late acting cytokines.

84. The method of claim 83, wherein said early acting cytokines are selected from the group consisting of stem cell factor, FLT3 ligand, interleukin-6, thrombopoietin and interleukin-3.

85. The method of claim 83, wherein said early acting cytokine is FLT3 ligand.

86. The method of claim 83, wherein said late acting cytokines are selected from the group consisting of granulocyte colony stimulating factor, granulocyte/macrophage colony stimulating factor and erythropoietin.

87. The method of claim 83, wherein said late acting cytokine is granulocyte colony stimulating factor.

88. The method of claim 73, wherein said stem cells are derived from a source selected from the group consisting of hematopoietic cells, neural cells, oligodendrocyte cells, skin cells, hepatic cells, embryonal stem cells, muscle cells, bone cells, mesenchymal cells, pancreatic cells, chondrocytes and stroma cells.

89. The method of claim 88, wherein said stem cells are derived from bone marrow or peripheral blood.

90. The method of claim 88, wherein said stem cells are derived from neonatal umbilical cord blood.

91. The method of claim 73, further comprising the step of selecting a population of stem cells enriched for hematopoietic stem cells.

92. The method of claim 91, wherein said selection is affected via CD34.

93. The method of claim 73, further comprising the step of selecting a population of stem cells enriched for early hematopoietic stem/progenitor cells.

94. The method of claim 93, wherein said selection is affected via CD133.

95. A method of mobilization of bone marrow stem cells into the peripheral blood of a donor for harvesting the cells comprising:
(a) administering to the donor an effective concentration of a a modulator of PI 3-kinase activity, said modulator selected capable of downregulating a PI 3-kinase activity or an expression of a gene encoding a PI 3-kinase, thereby expanding and inhibiting differentiation of a population of bone marrow stem cells; and
(b) harvesting the cells by leukopheresis.

96. A method of inhibiting maturation/differentiation of erythroid precursor cells for treatment of a β-hemoglobinopathic patient comprising administering to said patient an effective concentration of a modulator of PI 3-kinase activity, said modulator selected capable of downregulating a PI 3-kinase activity or an expression of a gene encoding a PI 3-kinase, thereby expanding and inhibiting differentiation of a population of stem cells of said patient such that upon removal of said modulator of PI 3-kinse from said patient, said stem cells undergo accelerated maturation resulting in elevated fetal hemoglobin production,
thereby ameliorating symptoms of β-hemoglobinopathy in said patient.

97. The method of 96, further comprising the step of administering to said patient a cytokine.

98. The method of claim 96, wherein said modulator selected capable of downregulating a PI 3-kinase activity or an expression of a gene encoding a PI 3-kinase is selected from the group consisting of:
(a) an inhibitor of PI 3-kinase catalytic activity;
(b) an antisense polynucleotide capable of specifically hybridizing with an mRNA transcript encoding a PI 3-kinase;
(c) a ribozyme which specifically cleaves PI 3-kinase transcripts, coding sequences and/or promoter elements;
(d) an siRNA molecule capable of inducing degradation of PI 3-kinase transcripts; and
(e) a DNAzyme which specifically cleaves PI 3-kinase transcripts or DNA.

99. The method of claim 96, wherein said inhibitor of PI 3-kinase activity is Wortmannin or LY294002.

100. The method of claim 96, wherein said modulator capable of downregulating PI 3-kinase activity or expression of a gene encoding PI 3-kinase is an anti-PI 3-kinase antibody.

101. The method of claim 100, wherein said anti-PI 3-kinase antibody is ScFV or Fab.

102. The method of claim 96, wherein said inhibitor of PI 3-kinase activity is an expressible polynucleotide encoding an anti-PI 3-kinase ScFv or Fab.

103. The method of claim 97, wherein said cytokine is selected from the group consisting of early acting cytokines and late acting cytokines.

104. The method of claim 103, wherein said early acting cytokines are selected from the group consisting of stem cell factor, FLT3 ligand, interleukin-6, thrombopoietin and interleukin-3.

105. The method of claim 103, wherein said early acting cytokine is FLT3 ligand.

106. The method of claim 103, wherein said late acting cytokines are selected from the group consisting of granulocyte colony stimulating factor, granulocyte/macrophage colony stimulating factor and erythropoietin.

107. The method of claim 103, wherein said late acting cytokine is granulocyte colony stimulating factor.

108. A method of preservation of undifferentiated stem cells comprising providing the undifferentiated stem cells with an effective concentration of a modulator of PI 3-kinase activity, said modulator selected capable of downregulating a PI 3-kinase activity or an expression of a gene encoding a PI 3-kinase of said undifferentiated stem cells, wherein said providing is performed in at least one of the steps of harvesting, isolating and storage of the undifferentiated hematopoietic cells.

109. The method of claim 108, wherein said modulator selected capable of downregulating a PI 3-kinase activity or an expression of a gene encoding a PI 3-kinase is selected from the group consisting of:
(a) an inhibitor of PI 3-kinase catalytic activity;
(b) an antisense polynucleotide capable of specifically hybridizing with an mRNA transcript encoding a PI 3-kinase;
(c) a ribozyme which specifically cleaves PI 3-kinase transcripts, coding sequences and/or promoter elements;
(d) an siRNA molecule capable of inducing degradation of PI 3-kinase transcripts; and
(e) a DNAzyme which specifically cleaves PI 3-kinase transcripts or DNA.

110. The method of claim 109, wherein said inhibitor of PI 3-kinase activity is Wortmannin or LY294002.

111. The method of claim 109, wherein said modulator capable of downregulating PI 3-kinase activity or expression of a gene encoding PI 3-kinase is an anti-PI 3-kinase antibody.

112. The method of claim 111, wherein said anti-PI 3-kinase antibody is ScFV or Fab.

113. The method of claim 109, wherein said providing is effected by transiently expressing said antisense polynucleotide, said ribozyme, said siRNA molecule or said DNAzyme within a stem cell.

114. The method of claim 109, wherein said providing is effected by
(a) providing an expressible polynucleotide capable of expressing said antisense polynucleotide, said ribozyme, said siRNA molecule or said DNAzyme, and
(b) stably integrating said expressible polynucleotide into a genome of a cell,
thereby providing a modulator capable of downregulating a PI 3-kinase activity or PI 3-kinase gene expression.

115. The method of claim 109, wherein said inhibitor of PI 3-kinase activity is an expressible polynucleotide encoding an anti-PI 3-kinase ScFv or Fab.

116. The method of claim 108, further comprising providing the cells with nutrients and cytokines.

117. The method of claim 116, wherein said cytokines are selected from the group consisting of early acting cytokines and late acting cytokines.

118. The method of claim 117, wherein said early acting cytokines are selected from the group consisting of stem cell factor, FLT3 ligand, interleukin-6, thrombopoietin and interleukin-3.

119. The method of claim 117, wherein said early acting cytokine is FLT3 ligand.

120. The method of claim 117, wherein said late acting cytokines are selected from the group consisting of granulocyte colony stimulating factor, granulocyte/macrophage colony stimulating factor and erythropoietin.

121. The method of claim 117, wherein said late acting cytokine is granulocyte colony stimulating factor.

122. The method of claim 108, wherein said stem cells are derived from a source selected from the group consisting of hematopoietic cells, neural cells, oligodendrocyte cells, skin cells, hepatic cells, embryonal stem cells, muscle cells, bone cells, mesenchymal cells, pancreatic cells, chondrocytes and stroma cells.

123. The method of claim 122, wherein said stem cells are derived from bone marrow or peripheral blood.

124. The method of claim 122, wherein said stem cells are derived from neonatal umbilical cord blood.

125. The method of claim 108, further comprising the step of selecting a population of stem cells enriched for hematopoietic stem cells.

126. The method of claim 125, wherein said selection is affected via CD34.

127. The method of claim 108, further comprising the step of selecting a population of stem cells enriched for early hematopoietic stem/progenitor cells.

128. The method of claim 127, wherein said selection is affected via CD133.

129. Stem cell collection bags, stem cell separation and stem cell washing buffers supplemented with an amount of a modulator of PI 3-kinase activity, said modulator selected capable of downregulating a PI 3-kinase activity or an expression of a gene encoding a PI 3-kinase, said amount sufficient to inhibit differentiation of a population of undifferentiated hematopoietic cells.

130. The stem cell collection bags and buffers of claim 129, wherein said modulator capable of downregulating PI 3-kinase activity or expression of a gene encoding PI 3-kinase is an inhibitor of PI 3-kinase activity.

131. The stem cell collection bags and buffers of claim 130, wherein said inhibitor of PI 3-kinase activity is Wortmannin or LY294002.

132. The stem cell collection bags and buffers of claim 129, wherein said modulator capable of downregulating PI 3-kinase activity or expression of a gene encoding PI 3-kinase is an anti-PI 3-kinase antibody.

133. The stem cell collection bags and buffers of claim 129, further supplemented with nutrients and cytokines.

134. The stem cell collection bags and buffers of claim 133, wherein said cytokines are selected from the group consisting of early acting cytokines and late acting cytokines.

135. The stem cell collection bags and buffers of claim 134, wherein said early acting cytokines are selected from the group consisting of stem cell factor, FLT3 ligand, interleukin-6, thrombopoietin and interleukin-3.

136. The stem cell collection bags and buffers of claim 134, wherein said early acting cytokine is FLT3 ligand.

137. The stem cell collection bags and buffers of claim 134, wherein said late acting cytokines are selected from the group consisting of granulocyte colony stimulating factor, granulocyte/macrophage colony stimulating factor and erythropoietin.

138. The stem cell collection bags and buffers of claim 134, wherein said late acting cytokine is granulocyte colony stimulating factor.

139. An assay for determining whether a modulator of PI 3-kinase activity is capable of inhibiting differentiation of cells, the assay comprising:
(a) culturing a population of cells capable of differentiating, in the presence or absence of said modulator of PI 3-kinase activity; and
(b) assessing changes in differentiation of said cells,
wherein an increase in differentiation as compared to untreated cells indicates a modulator of PI 3-kinase activity incapable of inhibiting differentiation, and whereas a lack of or decrease in differentiation as compared to untreated cells, indicates a modulator of PI 3-kinase activity capable of inhibiting differentiation.

140. The assay of claim 139, wherein said cells capable of differentiating are stem or progenitor cells, or substantially undifferentiated cells of a cell line.

141. The assay of claim 140, wherein said stem or progenitor cells are early hematopoietic and/or hematopoietic progenitor cells.

142. The assay of claim 139, further comprising providing the cells with nutrients and cytokines.

143. The assay of claim 142, wherein said cytokines are selected from the group consisting of early acting cytokines and late acting cytokines.

144. The assay of claim 140, wherein said stem cells are derived from a source selected from the group consisting of hematopoietic cells, neural cells, oligodendrocyte cells, skin cells, hepatic cells, embryonal stem cells, muscle cells, bone cells, mesenchymal cells, pancreatic cells, chondrocytes and stroma cells.

145. The assay of claim 139, wherein said assessing changes in differentiation is effected via differentiation markers.

146. The assay of claim 145, wherein said differentiation markers are selected from the group consisting of CD133, CD34, CD38, CD33, CD14, CD15, CD3, CD61 and CD19.

147. A method of ex vivo expanding and inhibiting differentiation of a population of stem cells, the method comprising:
(a) providing the cells ex vivo with conditions for cell proliferation;
(b) ex vivo reducing a capacity of said stem cells in responding to signaling pathways involving a PI 3-kinase activity;
thereby ex vivo expanding and inhibiting differentiation of the population of stem cells.
